(19) United States
(12) Patent Application Publication
(10) Pub. No.: US 2026/0285937 A1
(43) Pub. Date: Sep. 24, 2026

(54) CELL THERAPIES FOR TYPE 1 DIABETES

(71) Applicant: Abata Therapeutics, Inc., Watertown, MA (US)

(72) Inventors: Geetha MYLVAGANAM, Watertown, MA (US); Devapregasan MOODLEY, Watertown, MA (US); Jeremy BURNS, Watertown, MA (US); Yanbo ZHANG, Watertown, MA (US); Timothy NELSON, Watertown, MA (US)

(21) Appl. No.: 19/477,373

(22) PCT Filed: Apr. 19, 2024
(86) PCT No.: PCT/US2024/025443

§ 371 Date: Oct. 21, 2025

Related U.S. Application Data
(60) Provisional application No. 63/461,003, filed on Apr. 21, 2023.

(51) Int. Cl. C07K 14/705; A61K 40/11; A61K 40/32; A61K 40/41; C12N 5/0783; C12N 15/86

(52) U.S. Cl. CPC C07K 14/7051; A61K 40/11; A61K 40/32; A61K 40/41; C12N 5/0636; C12N 15/86; C12N 2510/00; C12N 2740/15043; C12N 2830/48

(57) ABSTRACT

The present disclosure is directed to T cell receptors targeting GAD65 and isolated cell populations comprising regulatory T cells exogenously expressing said TCRs used to treat Type 1 Diabetes.

### RELATED APPLICATIONS

This application claims the benefit under 35 U.S.C. § 119(e) of U.S. provisional application No. 63/461,003, filed on Apr. 21, 2023, the content of which is incorporated by reference herein in its entirety.

### REFERENCE TO AN ELECTRONIC SEQUENCE LISTING

The content of the electronic sequence listing (A133670007WO00-SEQ-MSB.xml; Size: 214,775 bytes; and Date of Creation: Apr. 17, 2024) is herein incorporated by reference in its entirety.

### BACKGROUND

Type 1 Diabetes is a T cell-mediated autoimmune disease resulting in islet β cell destruction, hypoinsulinemia, and altered glucose homeostasis. The activity of effector T cells and regulatory T cells, which jointly function in immune homeostasis, are dysregulated in patients having Type 1 Diabetes. It has been shown in the clinic that modulating T cells affects disease progression in these patients. There is evidence that therapeutic intervention in Type 1 Diabetes patients with the genetic HLA haplotype DR4 (over half of all patients), who are early in autoimmune pathogenesis, can prevent the onset of symptomatic disease and insulin dependence. Currently, curative treatments for Type 1 Diabetes do not exist, and available therapies rely on the treatment of symptoms often involving immunosuppressive reagents that can have severe side effects. Regulatory T cells have potential for the treatment of this disease and other autoimmune diseases because they can selectively target diseased cell types and tissues, generating a local immune response via an antigen-specific mechanism.

### SUMMARY

The present disclosure is based, at least in part, on the development of a number of T cell receptors (TCRs) specific to GAD65 that can be transduced into T cells (e.g., regulatory T cells) and expressed at the surface of those T cells. T cells expressing the TCRs of the disclosure are effectively activated in the presence of GAD65 when presented by antigen presenting cells. Accordingly, these TCRs provide a mechanism for directing T cells (e.g., regulatory T cells) to cells and tissues expressing GAD65 (e.g., for treatment of Type 1 Diabetes).

Some aspects of the disclosure provide one or more amino acid sequences encoding a TCR (e.g., an isolated or engineered TCR) that binds (e.g., specifically binds) to a GAD65 peptide complexed with a major histocompatibility complex (MHC) molecule. In some embodiments, the GAD65 peptide is GAD65 555-567 peptide (e.g., comprising the amino acid sequence of NFFRMVISNPAAT (SEQ ID NO: 109)).

Some aspects of the disclosure provide a TCR (e.g., an isolated or engineered TCR) that (a) comprises one or more amino acid sequences set forth in Table 1 (e.g., a CDR sequence belonging to any one of TCR-A, TCR-B, TCR-C, TCR-D, TCR-E, TCR-F, TCR-G, TCR-H, TCR-I, TCR-J, TCR-K, TCR-L, TCR-M, TCR-N, TCR-O, and TCR-P), and (b) binds (e.g., specifically binds) to a GAD65 peptide (e.g., GAD65 555-567 peptide) complexed with an MHC molecule. In some embodiments, the GAD65 peptide is GAD65 555-567 peptide.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 1, a CDR2-alpha sequence comprising the amino acid sequence of SEQ ID NO: 2, a CDR3-alpha sequence comprising the amino acid sequence of SEQ ID NO: 3, a CDR1-beta sequence comprising the amino acid sequence of SEQ ID NO: 6, a CDR2-beta sequence comprising the amino acid sequence of SEQ ID NO: 7, and/or a CDR3-beta sequence comprising the amino acid sequence of SEQ ID NO: 8.

In some embodiments, the TCR comprises a CDR1-alpha sequence comprising the amino acid sequence of SEQ ID NO: 1, a CDR2-alpha sequence comprising the amino acid sequence of SEQ ID NO: 2, and a CDR3-alpha sequence comprising the amino acid sequence of SEQ ID NO: 3. In some embodiments, the alpha chain comprises an alpha variable region comprising an amino acid sequence having at least 90%, 95%, or 100% identity to the amino acid sequence of SEQ ID NO: 4. In some embodiments, the alpha chain comprises an amino acid sequence having at least 90%, 95%, or 100% identity to the amino acid sequence of SEQ ID NO: 5. In some embodiments, the alpha chain comprises an amino acid sequence having at least 90%, 95%, or 100% identity to the amino acid sequence of SEQ ID NO: 260.

In some embodiments, the TCR comprises a CDR1-beta sequence comprising the amino acid sequence of SEQ ID NO: 6, a CDR2-beta sequence comprising the amino acid sequence of SEQ ID NO: 7, and a CDR3-beta sequence comprising the amino acid sequence of SEQ ID NO: 8. In some embodiments, the beta chain comprises a beta variable region comprising an amino acid sequence having at least 90%, 95%, or 100% identity to the amino acid sequence of SEQ ID NO: 9. In some embodiments, the beta chain comprises an amino acid sequence having at least 90%, 95%, or 100% identity to the amino acid sequence of SEQ ID NO: 10. In some embodiments, the beta chain comprises an amino acid sequence having at least 90%, 95%, or 100% identity to the amino acid sequence of SEQ ID NO: 261.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 11, a CDR2-alpha sequence comprising the amino acid sequence of SEQ ID NO: 12, a CDR3-alpha sequence comprising the amino acid sequence of SEQ ID NO: 13, a CDR1-beta sequence comprising the amino acid sequence of SEQ ID NO: 16, a CDR2-beta sequence comprising the amino acid sequence of SEQ ID NO: 17, and/or a CDR3-beta sequence comprising the amino acid sequence of SEQ ID NO: 18.

In some embodiments, the TCR comprises a CDR1-alpha sequence comprising the amino acid sequence of SEQ ID NO: 11, a CDR2-alpha sequence comprising the amino acid sequence of SEQ ID NO: 12, and a CDR3-alpha sequence comprising the amino acid sequence of SEQ ID NO: 13. In some embodiments, the alpha chain comprises an alpha variable region comprising an amino acid sequence having at least 90%, 95%, or 100% identity to the amino acid sequence of SEQ ID NO: 14. In some embodiments, the alpha chain comprises an amino acid sequence having at least 90%, 95%, or 100% identity to the amino acid sequence of SEQ ID NO: 15. In some embodiments, the alpha chain comprises an amino acid sequence having at least 90%, 95%, or 100% identity to the amino acid sequence of SEQ ID NO: 262.

In some embodiments, the TCR comprises a CDR1-beta sequence comprising the amino acid sequence of SEQ ID NO: 16, a CDR2-beta sequence comprising the amino acid sequence of SEQ ID NO: 17, and a CDR3-beta sequence comprising the amino acid sequence of SEQ ID NO: 18. In some embodiments, the beta chain comprises an alpha variable region comprising an amino acid sequence having at least 90%, 95%, or 100% identity to the amino acid sequence of SEQ ID NO: 19. In some embodiments, the beta chain comprises an amino acid sequence having at least 90%, 95%, or 100% identity to the amino acid sequence of SEQ ID NO: 20. In some embodiments, the beta chain comprises an amino acid sequence having at least 90%, 95%, or 100% identity to the amino acid sequence of SEQ ID NO: 263.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 21, a CDR2-alpha sequence comprising the amino acid sequence of SEQ ID NO: 22, a CDR3-alpha sequence comprising the amino acid sequence of SEQ ID NO: 23, a CDR1-beta sequence comprising the amino acid sequence of SEQ ID NO: 26, a CDR2-beta sequence comprising the amino acid sequence of SEQ ID NO: 27, and/or a CDR3-beta sequence comprising the amino acid sequence of SEQ ID NO: 28.

In some embodiments, the TCR comprises a CDR1-alpha sequence comprising the amino acid sequence of SEQ ID NO: 21, a CDR2-alpha sequence comprising the amino acid sequence of SEQ ID NO: 22, and a CDR3-alpha sequence comprising the amino acid sequence of SEQ ID NO: 23. In some embodiments, the alpha chain comprises an alpha variable region comprising an amino acid sequence having at least 90%, 95%, or 100% identity to the amino acid sequence of SEQ ID NO: 24. In some embodiments, the alpha chain comprises an amino acid sequence having at least 90%, 95%, or 100% identity to the amino acid sequence of SEQ ID NO: 25. In some embodiments, the alpha chain comprises an amino acid sequence having at least 90%, 95%, or 100% identity to the amino acid sequence of SEQ ID NO: 264.

In some embodiments, the TCR comprises a CDR1-beta sequence comprising the amino acid sequence of SEQ ID NO: 26, a CDR2-beta sequence comprising the amino acid sequence of SEQ ID NO: 27, and a CDR3-beta sequence comprising the amino acid sequence of SEQ ID NO: 28. In some embodiments, the beta chain comprises a beta variable region comprising an amino acid sequence having at least 90%, 95%, or 100% identity to the amino acid sequence of SEQ ID NO: 29. In some embodiments, the beta chain comprises an amino acid sequence having at least 90%, 95%, or 100% identity to the amino acid sequence of SEQ ID NO: 30. In some embodiments, the beta chain comprises an amino acid sequence having at least 90%, 95%, or 100% identity to the amino acid sequence of SEQ ID NO: 265.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises a CDR1-alpha sequence comprising the amino acid sequence of SEQ ID NO: 31, a CDR2-alpha sequence comprising the amino acid sequence of SEQ ID NO: 32, a CDR3-alpha sequence comprising the amino acid sequence of SEQ ID NO: 33, a CDR1-beta sequence comprising the amino acid sequence of SEQ ID NO: 36, a CDR2-beta sequence comprising the amino acid sequence of SEQ ID NO: 37, and/or a CDR3-beta sequence comprising the amino acid sequence of SEQ ID NO: 38.

In some embodiments, the TCR comprises a CDR1-alpha sequence comprising the amino acid sequence of SEQ ID NO: 31, a CDR2-alpha sequence comprising the amino acid sequence of SEQ ID NO: 32, and a CDR3-alpha sequence comprising the amino acid sequence of SEQ ID NO: 33. In some embodiments, the alpha chain comprises an alpha variable region comprising an amino acid sequence having at least 90%, 95%, or 100% identity to the amino acid sequence of SEQ ID NO: 34. In some embodiments, the alpha chain comprises an amino acid sequence having at least 90%, 95%, or 100% identity to the amino acid sequence of SEQ ID NO: 35. In some embodiments, the alpha chain comprises an amino acid sequence having at least 90%, 95%, or 100% identity to the amino acid sequence of SEQ ID NO: 266.

In some embodiments, the TCR comprises a CDR1-beta sequence comprising the amino acid sequence of SEQ ID NO: 36, a CDR2-beta sequence comprising the amino acid sequence of SEQ ID NO: 37, and a CDR3-beta sequence comprising the amino acid sequence of SEQ ID NO: 38. In some embodiments, the beta chain comprises a beta variable region comprising an amino acid sequence having at least 90%, 95%, or 100% identity to the amino acid sequence of SEQ ID NO: 39. In some embodiments, the beta chain comprises an amino acid sequence having at least 90%, 95%, or 100% identity to the amino acid sequence of SEQ ID NO: 40. In some embodiments, the beta chain comprises an amino acid sequence having at least 90%, 95%, or 100% identity to the amino acid sequence of SEQ ID NO: 267.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 41, a CDR2-alpha sequence comprising the amino acid sequence of SEQ ID NO: 42, a CDR3-alpha sequence comprising the amino acid sequence of SEQ ID NO: 43, a CDR1-beta sequence comprising the amino acid sequence of SEQ ID NO: 46, a CDR2-beta sequence comprising the amino acid sequence of SEQ ID NO: 47, and/or a CDR3-beta sequence comprising the amino acid sequence of SEQ ID NO: 48.

In some embodiments, the TCR comprises a CDR1-alpha sequence comprising the amino acid sequence of SEQ ID NO: 41, a CDR2-alpha sequence comprising the amino acid sequence of SEQ ID NO: 42, and a CDR3-alpha sequence comprising the amino acid sequence of SEQ ID NO: 43. In some embodiments, the alpha chain comprises an alpha variable region comprising an amino acid sequence having at least 90%, 95%, or 100% identity to the amino acid sequence of SEQ ID NO: 44. In some embodiments, the alpha chain comprises an amino acid sequence having at least 90%, 95%, or 100% identity to the amino acid sequence of SEQ ID NO: 45. In some embodiments, the alpha chain comprises an amino acid sequence having at least 90%, 95%, or 100% identity to the amino acid sequence of SEQ ID NO: 268.

In some embodiments, the TCR comprises a CDR1-beta sequence comprising the amino acid sequence of SEQ ID NO: 46, a CDR2-beta sequence comprising the amino acid sequence of SEQ ID NO: 47, and a CDR3-beta sequence comprising the amino acid sequence of SEQ ID NO: 48. In some embodiments, the beta chain comprises a beta variable region comprising an amino acid sequence having at least 90%, 95%, or 100% identity to the amino acid sequence of SEQ ID NO: 49. In some embodiments, the beta chain comprises an amino acid sequence having at least 90%, 95%, or 100% identity to the amino acid sequence of SEQ ID NO: 50. In some embodiments, the beta chain comprises an amino acid sequence having at least 90%, 95%, or 100% identity to the amino acid sequence of SEQ ID NO: 269.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 51, a CDR2-alpha sequence comprising the amino acid sequence of SEQ ID NO: 52, a CDR3-alpha sequence comprising the amino acid sequence of SEQ ID NO: 53, a CDR1-beta sequence comprising the amino acid sequence of SEQ ID NO: 56, a CDR2-beta sequence comprising the amino acid sequence of SEQ ID NO: 57, and/or a CDR3-beta sequence comprising the amino acid sequence of SEQ ID NO: 58.

In some embodiments, the TCR comprises a CDR1-alpha sequence comprising the amino acid sequence of SEQ ID NO: 51, a CDR2-alpha sequence comprising the amino acid sequence of SEQ ID NO: 52, and a CDR3-alpha sequence comprising the amino acid sequence of SEQ ID NO: 53. In some embodiments, the alpha chain comprises an alpha variable region comprising an amino acid sequence having at least 90%, 95%, or 100% identity to the amino acid sequence of SEQ ID NO: 54. In some embodiments, the alpha chain comprises an amino acid sequence having at least 90%, 95%, or 100% identity to the amino acid sequence of SEQ ID NO: 55. In some embodiments, the alpha chain comprises an amino acid sequence having at least 90%, 95%, or 100% identity to the amino acid sequence of SEQ ID NO: 270.

In some embodiments, the TCR comprises a CDR1-beta sequence comprising the amino acid sequence of SEQ ID NO: 56, a CDR2-beta sequence comprising the amino acid sequence of SEQ ID NO: 57, and a CDR3-beta sequence comprising the amino acid sequence of SEQ ID NO: 58. In some embodiments, the beta chain comprises a beta variable region comprising an amino acid sequence having at least 90%, 95%, or 100% identity to the amino acid sequence of SEQ ID NO: 59. In some embodiments, the beta chain comprises an amino acid sequence having at least 90%, 95%, or 100% identity to the amino acid sequence of SEQ ID NO: 60. In some embodiments, the beta chain comprises an amino acid sequence having at least 90%, 95%, or 100% identity to the amino acid sequence of SEQ ID NO: 271.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 61, a CDR2-alpha sequence comprising the amino acid sequence of SEQ ID NO: 62, a CDR3-alpha sequence comprising the amino acid sequence of SEQ ID NO: 63, a CDR1-beta sequence comprising the amino acid sequence of SEQ ID NO: 66, a CDR2-beta sequence comprising the amino acid sequence of SEQ ID NO: 67, and/or a CDR3-beta sequence comprising the amino acid sequence of SEQ ID NO: 68.

In some embodiments, the TCR comprises a CDR1-alpha sequence comprising the amino acid sequence of SEQ ID NO: 61, a CDR2-alpha sequence comprising the amino acid sequence of SEQ ID NO: 62, and a CDR3-alpha sequence comprising the amino acid sequence of SEQ ID NO: 63. In some embodiments, the alpha chain comprises an alpha variable region comprising an amino acid sequence having at least 90%, 95%, or 100% identity to the amino acid sequence of SEQ ID NO: 64. In some embodiments, the alpha chain comprises an amino acid sequence having at least 90%, 95%, or 100% identity to the amino acid sequence of SEQ ID NO: 65. In some embodiments, the alpha chain comprises an amino acid sequence having at least 90%, 95%, or 100% identity to the amino acid sequence of SEQ ID NO: 272.

In some embodiments, the TCR comprises a CDR1-beta sequence comprising the amino acid sequence of SEQ ID NO: 66, a CDR2-beta sequence comprising the amino acid sequence of SEQ ID NO: 67, and a CDR3-beta sequence comprising the amino acid sequence of SEQ ID NO: 68. In some embodiments, the beta chain comprises a beta variable region comprising an amino acid sequence having at least 90%, 95%, or 100% identity to the amino acid sequence of SEQ ID NO: 69. In some embodiments, the beta chain comprises an amino acid sequence having at least 90%, 95%, or 100% identity to the amino acid sequence of SEQ ID NO: 70. In some embodiments, the beta chain comprises an amino acid sequence having at least 90%, 95%, or 100% identity to the amino acid sequence of SEQ ID NO: 273.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 71, a CDR2-alpha sequence comprising the amino acid sequence of SEQ ID NO: 72, a CDR3-alpha sequence comprising the amino acid sequence of SEQ ID NO: 73, a CDR1-beta sequence comprising the amino acid sequence of SEQ ID NO: 76, a CDR2-beta sequence comprising the amino acid sequence of SEQ ID NO: 77, and/or a CDR3-beta sequence comprising the amino acid sequence of SEQ ID NO: 78.

In some embodiments, the TCR comprises a CDR1-alpha sequence comprising the amino acid sequence of SEQ ID NO: 71, a CDR2-alpha sequence comprising the amino acid sequence of SEQ ID NO: 72, and a CDR3-alpha sequence comprising the amino acid sequence of SEQ ID NO: 73. In some embodiments, the alpha chain comprises an alpha variable region comprising an amino acid sequence having at least 90%, 95%, or 100% identity to the amino acid sequence of SEQ ID NO: 74. In some embodiments, the alpha chain comprises an amino acid sequence having at least 90%, 95%, or 100% identity to the amino acid sequence of SEQ ID NO: 75. In some embodiments, the alpha chain comprises an amino acid sequence having at least 90%, 95%, or 100% identity to the amino acid sequence of SEQ ID NO: 274.

In some embodiments, the TCR comprises a CDR1-beta sequence comprising the amino acid sequence of SEQ ID NO: 76, a CDR2-beta sequence comprising the amino acid sequence of SEQ ID NO: 77, and a CDR3-beta sequence comprising the amino acid sequence of SEQ ID NO: 78. In some embodiments, the beta chain comprises a beta variable region comprising an amino acid sequence having at least 90%, 95%, or 100% identity to the amino acid sequence of SEQ ID NO: 79. In some embodiments, the beta chain comprises an amino acid sequence having at least 90%, 95%, or 100% identity to the amino acid sequence of SEQ ID NO: 80. In some embodiments, the beta chain comprises an amino acid sequence having at least 90%, 95%, or 100% identity to the amino acid sequence of SEQ ID NO: 275.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 81, a CDR2-alpha sequence comprising the amino acid sequence of SEQ ID NO: 82, a CDR3-alpha sequence comprising the amino acid sequence of SEQ ID NO: 83, a CDR1-beta sequence comprising the amino acid sequence of SEQ ID NO: 86, a CDR2-beta sequence comprising the amino acid sequence of SEQ ID NO: 87, and/or a CDR3-beta sequence comprising the amino acid sequence of SEQ ID NO: 88.

In some embodiments, the TCR comprises a CDR1-alpha sequence comprising the amino acid sequence of SEQ ID NO: 81, a CDR2-alpha sequence comprising the amino acid sequence of SEQ ID NO: 82, and a CDR3-alpha sequence comprising the amino acid sequence of SEQ ID NO: 83. In some embodiments, the alpha chain comprises an alpha variable region comprising an amino acid sequence having at least 90%, 95%, or 100% identity to the amino acid sequence of SEQ ID NO: 84. In some embodiments, the alpha chain comprises an amino acid sequence having at least 90%, 95%, or 100% identity to the amino acid sequence of SEQ ID NO: 85. In some embodiments, the alpha chain comprises an amino acid sequence having at least 90%, 95%, or 100% identity to the amino acid sequence of SEQ ID NO: 276.

In some embodiments, the TCR comprises a CDR1-beta sequence comprising the amino acid sequence of SEQ ID NO: 86, a CDR2-beta sequence comprising the amino acid sequence of SEQ ID NO: 87, and a CDR3-beta sequence comprising the amino acid sequence of SEQ ID NO: 88. In some embodiments, the beta chain comprises a beta variable region comprising an amino acid sequence having at least 90%, 95%, or 100% identity to the amino acid sequence of SEQ ID NO: 89. In some embodiments, the beta chain comprises an amino acid sequence having at least 90%, 95%, or 100% identity to the amino acid sequence of SEQ ID NO: 90. In some embodiments, the beta chain comprises an amino acid sequence having at least 90%, 95%, or 100% identity to the amino acid sequence of SEQ ID NO: 277.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 91, a CDR2-alpha sequence comprising the amino acid sequence of SEQ ID NO: 92, a CDR3-alpha sequence comprising the amino acid sequence of SEQ ID NO: 93, a CDR1-beta sequence comprising the amino acid sequence of SEQ ID NO: 96, a CDR2-beta sequence comprising the amino acid sequence of SEQ ID NO: 97, and/or a CDR3-beta sequence comprising the amino acid sequence of SEQ ID NO: 98.

In some embodiments, the TCR comprises a CDR1-alpha sequence comprising the amino acid sequence of SEQ ID NO: 91, a CDR2-alpha sequence comprising the amino acid sequence of SEQ ID NO: 92, and a CDR3-alpha sequence comprising the amino acid sequence of SEQ ID NO: 93. In some embodiments, the alpha chain comprises an alpha variable region comprising an amino acid sequence having at least 90%, 95%, or 100% identity to the amino acid sequence of SEQ ID NO: 94. In some embodiments, the alpha chain comprises an amino acid sequence having at least 90%, 95%, or 100% identity to the amino acid sequence of SEQ ID NO: 95. In some embodiments, the alpha chain comprises an amino acid sequence having at least 90%, 95%, or 100% identity to the amino acid sequence of SEQ ID NO: 278.

In some embodiments, the TCR comprises a CDR1-beta sequence comprising the amino acid sequence of SEQ ID NO: 96, a CDR2-beta sequence comprising the amino acid sequence of SEQ ID NO: 97, and a CDR3-beta sequence comprising the amino acid sequence of SEQ ID NO: 98. In some embodiments, the beta chain comprises a beta variable region comprising an amino acid sequence having at least 90%, 95%, or 100% identity to the amino acid sequence of SEQ ID NO: 99. In some embodiments, the beta chain comprises an amino acid sequence having at least 90%, 95%, or 100% identity to the amino acid sequence of SEQ ID NO: 100. In some embodiments, the beta chain comprises an amino acid sequence having at least 90%, 95%, or 100% identity to the amino acid sequence of SEQ ID NO: 279.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 110, a CDR2-alpha sequence comprising the amino acid sequence of SEQ ID NO: 111, a CDR3-alpha sequence comprising the amino acid sequence of SEQ ID NO: 112, a CDR1-beta sequence comprising the amino acid sequence of SEQ ID NO: 115, a CDR2-beta sequence comprising the amino acid sequence of SEQ ID NO: 116, and/or a CDR3-beta sequence comprising the amino acid sequence of SEQ ID NO: 117.

In some embodiments, the TCR comprises a CDR1-alpha sequence comprising the amino acid sequence of SEQ ID NO: 110, a CDR2-alpha sequence comprising the amino acid sequence of SEQ ID NO: 111, and a CDR3-alpha sequence comprising the amino acid sequence of SEQ ID NO: 112. In some embodiments, the alpha chain comprises an alpha variable region comprising an amino acid sequence having at least 90%, 95%, or 100% identity to the amino acid sequence of SEQ ID NO: 113. In some embodiments, the alpha chain comprises an amino acid sequence having at least 90%, 95%, or 100% identity to the amino acid sequence of SEQ ID NO: 114. In some embodiments, the alpha chain comprises an amino acid sequence having at least 90%, 95%, or 100% identity to the amino acid sequence of SEQ ID NO: 280.

In some embodiments, the TCR comprises a CDR1-beta sequence comprising the amino acid sequence of SEQ ID NO: 115, a CDR2-beta sequence comprising the amino acid sequence of SEQ ID NO: 116, and a CDR3-beta sequence comprising the amino acid sequence of SEQ ID NO: 117. In some embodiments, the beta chain comprises a beta variable region comprising an amino acid sequence having at least 90%, 95%, or 100% identity to the amino acid sequence of SEQ ID NO: 118. In some embodiments, the beta chain comprises an amino acid sequence having at least 90%, 95%, or 100% identity to the amino acid sequence of SEQ ID NO: 119. In some embodiments, the beta chain comprises an amino acid sequence having at least 90%, 95%, or 100% identity to the amino acid sequence of SEQ ID NO: 281.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 120, a CDR2-alpha sequence comprising the amino acid sequence of SEQ ID NO: 121, a CDR3-alpha sequence comprising the amino acid sequence of SEQ ID NO: 122, a CDR1-beta sequence comprising the amino acid sequence of SEQ ID NO: 125, a CDR2-beta sequence comprising the amino acid sequence of SEQ ID NO: 126, and/or a CDR3-beta sequence comprising the amino acid sequence of SEQ ID NO: 127.

In some embodiments, the TCR comprises a CDR1-alpha sequence comprising the amino acid sequence of SEQ ID NO: 120, a CDR2-alpha sequence comprising the amino acid sequence of SEQ ID NO: 121, and a CDR3-alpha sequence comprising the amino acid sequence of SEQ ID NO: 122. In some embodiments, the alpha chain comprises an alpha variable region comprising an amino acid sequence having at least 90%, 95%, or 100% identity to the amino acid sequence of SEQ ID NO: 123. In some embodiments, the alpha chain comprises an amino acid sequence having at least 90%, 95%, or 100% identity to the amino acid sequence of SEQ ID NO: 124. In some embodiments, the alpha chain comprises an amino acid sequence having at least 90%, 95%, or 100% identity to the amino acid sequence of SEQ ID NO: 282.

In some embodiments, the TCR comprises a CDR1-beta sequence comprising the amino acid sequence of SEQ ID NO: 125, a CDR2-beta sequence comprising the amino acid sequence of SEQ ID NO: 126, and a CDR3-beta sequence comprising the amino acid sequence of SEQ ID NO: 127. In some embodiments, the beta chain comprises a beta variable region comprising an amino acid sequence having at least 90%, 95%, or 100% identity to the amino acid sequence of SEQ ID NO: 128. In some embodiments, the beta chain comprises an amino acid sequence having at least 90%, 95%, or 100% identity to the amino acid sequence of SEQ ID NO: 129. In some embodiments, the beta chain comprises an amino acid sequence having at least 90%, 95%, or 100% identity to the amino acid sequence of SEQ ID NO: 283.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 130, a CDR2-alpha sequence comprising the amino acid sequence of SEQ ID NO: 131, a CDR3-alpha sequence comprising the amino acid sequence of SEQ ID NO: 132, a CDR1-beta sequence comprising the amino acid sequence of SEQ ID NO: 135, a CDR2-beta sequence comprising the amino acid sequence of SEQ ID NO: 136, and/or a CDR3-beta sequence comprising the amino acid sequence of SEQ ID NO: 137.

In some embodiments, the TCR comprises a CDR1-alpha sequence comprising the amino acid sequence of SEQ ID NO: 130, a CDR2-alpha sequence comprising the amino acid sequence of SEQ ID NO: 131, and a CDR3-alpha sequence comprising the amino acid sequence of SEQ ID NO: 132. In some embodiments, the alpha chain comprises an alpha variable region comprising an amino acid sequence having at least 90%, 95%, or 100% identity to the amino acid sequence of SEQ ID NO: 133. In some embodiments, the alpha chain comprises an amino acid sequence having at least 90%, 95%, or 100% identity to the amino acid sequence of SEQ ID NO: 134. In some embodiments, the alpha chain comprises an amino acid sequence having at least 90%, 95%, or 100% identity to the amino acid sequence of SEQ ID NO: 284.

In some embodiments, the TCR comprises a CDR1-beta sequence comprising the amino acid sequence of SEQ ID NO: 135, a CDR2-beta sequence comprising the amino acid sequence of SEQ ID NO: 136, and a CDR3-beta sequence comprising the amino acid sequence of SEQ ID NO: 137. In some embodiments, the beta chain comprises a beta variable region comprising an amino acid sequence having at least 90%, 95%, or 100% identity to the amino acid sequence of SEQ ID NO: 138. In some embodiments, the beta chain comprises an amino acid sequence having at least 90%, 95%, or 100% identity to the amino acid sequence of SEQ ID NO: 139. In some embodiments, the beta chain comprises an amino acid sequence having at least 90%, 95%, or 100% identity to the amino acid sequence of SEQ ID NO: 285.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 140, a CDR2-alpha sequence comprising the amino acid sequence of SEQ ID NO: 141, a CDR3-alpha sequence comprising the amino acid sequence of SEQ ID NO: 142, a CDR1-beta sequence comprising the amino acid sequence of SEQ ID NO: 145, a CDR2-beta sequence comprising the amino acid sequence of SEQ ID NO: 146, and/or a CDR3-beta sequence comprising the amino acid sequence of SEQ ID NO: 147.

In some embodiments, the TCR comprises a CDR1-alpha sequence comprising the amino acid sequence of SEQ ID NO: 140, a CDR2-alpha sequence comprising the amino acid sequence of SEQ ID NO: 141, and a CDR3-alpha sequence comprising the amino acid sequence of SEQ ID NO: 142. In some embodiments, the alpha chain comprises an alpha variable region comprising an amino acid sequence having at least 90%, 95%, or 100% identity to the amino acid sequence of SEQ ID NO: 143. In some embodiments, the alpha chain comprises an amino acid sequence having at least 90%, 95%, or 100% identity to the amino acid sequence of SEQ ID NO: 144. In some embodiments, the alpha chain comprises an amino acid sequence having at least 90%, 95%, or 100% identity to the amino acid sequence of SEQ ID NO: 286.

In some embodiments, the TCR comprises a CDR1-beta sequence comprising the amino acid sequence of SEQ ID NO: 145, a CDR2-beta sequence comprising the amino acid sequence of SEQ ID NO: 146, and a CDR3-beta sequence comprising the amino acid sequence of SEQ ID NO: 147. In some embodiments, the beta chain comprises a beta variable region comprising an amino acid sequence having at least 90%, 95%, or 100% identity to the amino acid sequence of SEQ ID NO: 148. In some embodiments, the beta chain comprises an amino acid sequence having at least 90%, 95%, or 100% identity to the amino acid sequence of SEQ ID NO: 149. In some embodiments, the beta chain comprises an amino acid sequence having at least 90%, 95%, or 100% identity to the amino acid sequence of SEQ ID NO: 287.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 150, a CDR2-alpha sequence comprising the amino acid sequence of SEQ ID NO: 151, a CDR3-alpha sequence comprising the amino acid sequence of SEQ ID NO: 152, a CDR1-beta sequence comprising the amino acid sequence of SEQ ID NO: 155, a CDR2-beta sequence comprising the amino acid sequence of SEQ ID NO: 156, and/or a CDR3-beta sequence comprising the amino acid sequence of SEQ ID NO: 157.

In some embodiments, the TCR comprises aa CDR1-alpha sequence comprising the amino acid sequence of SEQ ID NO: 150, a CDR2-alpha sequence comprising the amino acid sequence of SEQ ID NO: 151, and a CDR3-alpha sequence comprising the amino acid sequence of SEQ ID NO: 152. In some embodiments, the alpha chain comprises an alpha variable region comprising an amino acid sequence having at least 90%, 95%, or 100% identity to the amino acid sequence of SEQ ID NO: 153. In some embodiments, the alpha chain comprises an amino acid sequence having at least 90%, 95%, or 100% identity to the amino acid sequence of SEQ ID NO: 154. In some embodiments, the alpha chain comprises an amino acid sequence having at least 90%, 95%, or 100% identity to the amino acid sequence of SEQ ID NO: 288.

In some embodiments, the TCR comprises a CDR1-beta sequence comprising the amino acid sequence of SEQ ID NO: 155, a CDR2-beta sequence comprising the amino acid sequence of SEQ ID NO: 156, and a CDR3-beta sequence comprising the amino acid sequence of SEQ ID NO: 157. In some embodiments, the beta chain comprises a beta variable region comprising an amino acid sequence having at least 90%, 95%, or 100% identity to the amino acid sequence of SEQ ID NO: 158. In some embodiments, the beta chain comprises an amino acid sequence having at least 90%, 95%, or 100% identity to the amino acid sequence of SEQ ID NO: 159. In some embodiments, the beta chain comprises an amino acid sequence having at least 90%, 95%, or 100% identity to the amino acid sequence of SEQ ID NO: 289.

Some aspects relate to a TCR (e.g., an isolated or engineered TCR) that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 160, a CDR2-alpha sequence comprising the amino acid sequence of SEQ ID NO: 161, a CDR3-alpha sequence comprising the amino acid sequence of SEQ ID NO: 162, a CDR1-beta sequence comprising the amino acid sequence of SEQ ID NO: 165, a CDR2-beta sequence comprising the amino acid sequence of SEQ ID NO: 166, and/or a CDR3-beta sequence comprising the amino acid sequence of SEQ ID NO: 167.

In some embodiments, the TCR comprises a CDR1-alpha sequence comprising the amino acid sequence of SEQ ID NO: 160, a CDR2-alpha sequence comprising the amino acid sequence of SEQ ID NO: 161, and a CDR3-alpha sequence comprising the amino acid sequence of SEQ ID NO: 162. In some embodiments, the alpha chain comprises an alpha variable region comprising an amino acid sequence having at least 90%, 95%, or 100% identity to the amino acid sequence of SEQ ID NO: 163. In some embodiments, the alpha chain comprises an amino acid sequence having at least 90%, 95%, or 100% identity to the amino acid sequence of SEQ ID NO: 164. In some embodiments, the alpha chain comprises an amino acid sequence having at least 90%, 95%, or 100% identity to the amino acid sequence of SEQ ID NO: 290.

In some embodiments, the TCR comprises a CDR1-beta sequence comprising the amino acid sequence of SEQ ID NO: 165, a CDR2-beta sequence comprising the amino acid sequence of SEQ ID NO: 166, and a CDR3-beta sequence comprising the amino acid sequence of SEQ ID NO: 167. In some embodiments, the beta chain comprises a beta variable region comprising an amino acid sequence having at least 90%, 95%, or 100% identity to the amino acid sequence of SEQ ID NO: 168. In some embodiments, the beta chain comprises an amino acid sequence having at least 90%, 95%, or 100% identity to the amino acid sequence of SEQ ID NO: 169. In some embodiments, the beta chain comprises an amino acid sequence having at least 90%, 95%, or 100% identity to the amino acid sequence of SEQ ID NO: 291.

In some embodiments, the GAD65 peptide is a GAD65 555-567 peptide comprising the amino acid sequence of NFFRMVISNPAAT (SEQ ID NO: 109). In some embodiments, the MHC is a class II MHC. In some embodiments, the class II MHC comprises HLA-DRB1*04:01.

In some embodiments, the TCR is encoded as a single polypeptide, optionally wherein the polypeptide comprises an N-terminal beta domain and a C-terminal alpha domain. In some embodiments, the single polypeptide comprises a self-cleaving peptide sequence positioned between the alpha chain and the beta chain. In some embodiments, the self-cleaving peptide sequence is a 2A peptide sequence, optionally wherein the 2A peptide sequence is a P2A, E2A, F2A, or T2A peptide sequence.

In some embodiments, the alpha chain comprises an alpha constant region comprising an amino acid sequence having at least 90%, 95%, or 100% identity to the amino acid sequence of SEQ ID NO: 106. In some embodiments, the beta chain comprises a beta constant region comprising an amino acid sequence having at least 90%, 95%, or 100% identity to the amino acid sequence of SEQ ID NO: 107 or 108.

In some embodiments, the alpha chain comprises a murine constant region. In some embodiments, the beta chain comprises a murine constant region.

In some embodiments, the TCR comprises one or more amino acid substitutions to cysteine residues in the alpha chain constant region and the beta chain constant region, and wherein the cysteine residues are capable of forming one or more disulfide bridges. In some embodiments, the alpha chain constant region comprises a T48C amino acid substitution relative to an alpha chain constant region comprising the amino acid sequence of SEQ ID NO: 106, and wherein the beta chain constant region comprises a S57C amino acid substitution relative to a beta chain constant region comprising the amino acid sequence of SEQ ID NO: 108.

Some aspects of disclosure provide a nucleic acid encoding any one of the TCRs described herein. In some embodiments, the nucleic acid is a vector (e.g., a viral vector or a lentiviral vector).

In some embodiments, the nucleic acid comprises a promoter operably linked to a coding sequence encoding the TCR, optionally wherein the promoter is an EF-1 alpha promoter or an MND promoter. In some embodiments, the nucleic acid further comprises an enhancer element, optionally an optimized post-transcriptional regulatory element (oPRE) or a woodchuck hepatitis virus post-transcriptional regulatory element (WPRE), further optionally WPRE-mut6. In some embodiments, the coding sequence is codon-optimized.

Some aspects of the disclosure provide an engineered cell comprising a TCR (e.g., engineered TCR) that binds (e.g., specifically binds) to a GAD65 peptide (e.g., GAD65 555-567 peptide) complexed with a major histocompatibility complex (MHC) molecule. In some embodiments, the GAD65 peptide is GAD65 555-567 peptide. In some embodiments, the TCR is any one of the TCRs described herein. In some embodiments, the engineered cell is a T cell, optionally a regulatory T cell. In some embodiments, the engineered cell is derived from a subject, optionally a subject having or suspected of having Type 1 Diabetes.

Some aspects of the disclosure provide a cell population comprising a plurality of engineered cells (e.g., regulatory T cells) comprising any one of the TCRs described herein.

Some aspects of the disclosure provide a pharmaceutical composition comprising a cell population described herein and a pharmaceutically acceptable excipient. Other aspects of the disclosure provide a composition comprising a cell population described herein and a cryopreservative.

Some aspects of the disclosure provide a method comprising administering to a subject a population of cells, a pharmaceutical composition, or a composition described herein, wherein the subject has Type 1 Diabetes. In some embodiments, the population of cells, the pharmaceutical composition, or the composition is administered in an effective amount to alleviate one or more symptom of Type 1 Diabetes. In some embodiments, the administering comprises intravenous administration. In some embodiments, the administering comprises one or more infusion.

### BRIEF DESCRIPTION OF THE DRAWINGS

FIG. 1 provides graphs showing activation of TCR-null Jurkat cells by T cell receptors (TCRs) of the disclosure as determined by CD69 levels at the concentrations of GAD65 peptide shown, where the TCRs either have unmodified or disulfide-modified (DS) constant domain sequences.

FIG. 2 provides graphs showing activation of conventional CD4⁺ T cells by TCRs of the disclosure as determined by CD69 levels at the concentrations of GAD65 peptide shown, where the TCRs either have unmodified or disulfide-modified (DS) constant domain sequences.

FIGS. 3A-3C provide graphs showing the activity of regulatory T cells (Tregs) expressing TCRs of the disclosure to suppress proliferation of conventional T cells (Tconv) and expression of activation associated markers (CD71 and CD25) following co-culture at the indicated Treg:Tconv ratios in the presence of GAD65 555-567 peptide presented by HLA-DRB1*04:01⁺ peripheral blood mononuclear cells (PBMCs).

### DETAILED DESCRIPTION

The present disclosure provides compositions of T cell receptors (TCRs), compositions of engineered regulatory T cells comprising said TCRs, and methods of using the engineered regulatory T cells for treatment of Type 1 Diabetes. The TCRs are capable of specifically binding to GAD65 peptides presented by a major histocompatibility complex (MHC) molecule (e.g., MHC class II molecule). Engineering of regulatory T cells to express these TCRs allows the T cells to specifically target discrete cell types and tissues associated with Type 1 Diabetes in order to prevent immune-mediated destruction, restore homeostasis, and promote repair in affected tissues.

While many of the aspects and embodiments described herein are directed to regulatory T cells, the TCRs of the disclosure may be expressed in other T cell types, depending on the intended use of the T cell expressing TCR (e.g., for targeting the T cells to a GAD65 peptide, such as GAD65 555-567 peptide).

### T Cell Receptors

In some embodiments, the disclosure provides a T cell receptor (TCR) that allows for antigen-specific targeting (e.g., by engineered regulatory T cells). A TCR is a transmembrane heterodimer that includes an alpha chain and beta chain linked by a disulfide bond. Within these chains are complementary determining regions (CDRs) that determine the target peptide to which the TCR will bind. TCRs activate T cells in which they reside leading to a plethora of immune responses. Antigen presenting cells digest certain proteins (antigens) and display their fragments (peptides) on major histocompatibility complexes (MHC). This peptide-MHC (pMHC) complex binds to the TCR while other co-stimulatory molecules are activated leading to T cell activation, proliferation, differentiation, apoptosis, or cytokine release.

A TCR binds specifically to a target peptide (e.g., a GAD65 peptide) complexed with an MHC molecule (e.g., a class II MHC molecule). A TCR is considered to bind “specifically” to a target peptide complexed with an MHC molecule if the TCR has a higher binding affinity for the target peptide complexed with an MHC molecule relative to a non-target peptide complexed with an MHC molecule. A TCR may bind to a target peptide complexed with an MHC molecule with a binding affinity of at least 10⁻⁴ M, 10⁻⁵ M, 10⁻⁶ M, 10⁻⁷ M, 10⁻⁸ M, 10⁻⁹ M, or 10⁻¹⁰ M (e.g., 10⁻⁴ M to 10⁻¹⁰ M). In some embodiments, a TCR is considered to bind “specifically” to a target peptide complexed with an MHC molecule if a T cell expressing the TCR becomes activated (e.g., as assessed by increased CD69 expression) when contacted with the target peptide complexed with an MHC molecule, or becomes more highly activated relative to a non-target peptide complexed with an MHC molecule. In some embodiments, the peptide is presented by a cell expressing the MHC molecule. In some embodiments, the TCR binds specifically to a GAD65 peptide. In some embodiments, the TCR binds specifically to a GAD65 555-567 peptide (e.g., comprising the amino acid sequence of SEQ ID NO: 109).

In some embodiments, the TCR comprises one or more amino acid sequences as described in Table 1 (e.g., one or more amino acid sequences belonging to any one of TCR-A, TCR-B, TCR-C, TCR-D, TCR-E, TCR-F, TCR-G, TCR-H, TCR-I, TCR-J, TCR-K, TCR-L, TCR-M, TCR-N, TCR-O, or TCR-P,).

A TCR may comprise the amino acid sequence of any alpha chain CDR1, CDR2, or CDR3 as provided in Table 1. In some embodiments, the alpha CDR1 of the TCR is any one of SEQ ID NOs: 1, 11, 21, 31, 41, 51, 61, 71, 81, 91, 110, 120, 130, 140, 150, 160. In some embodiments, the alpha CDR2 of the TCR is any one of SEQ ID NOs: 2, 12, 22, 32, 42, 52, 62, 72, 82, 92, 111, 121, 131, 141, 151, 161. In some embodiments, the alpha CDR3 of the TCR is any one of SEQ ID NOs: 3, 13, 23, 33, 43, 53, 63, 73, 83, 93, 112, 122, 132, 142, 152, 162. A TCR may comprise the amino acid sequence of any beta chain CDR1, CDR2, or CDR3 as provided in Table 1. In some embodiments, the beta CDR1 of the TCR is any one of SEQ ID NOs: 6, 16, 26, 36, 46, 56, 66, 76, 86, 96, 115, 125, 135, 145, 155, 165. In some embodiments, the beta CDR2 of the TCR is any one of SEQ ID NOs: 7, 17, 27, 37, 47, 57, 67, 77, 87, 97, 116, 126, 136, 146, 156, 166. In some embodiments, the beta CDR3 of the TCR is any one of SEQ ID NOs: 8, 18, 28, 38, 48, 58, 68, 78, 88, 98, 117, 127, 137, 147, 157, 167.

In some embodiments, the alpha chain variable region of the TCR comprises an amino acid sequence having at least 90%, 95%, or 100% identity to the amino acid sequence of SEQ ID NO: 4, 14, 24, 34, 44, 54, 64, 74, 84, 94, 113, 123, 133, 143, 153, 163. In some embodiments, the beta chain variable region of the TCR comprises an amino acid sequence having at least 90%, 95%, or 100% identity to the amino acid sequence of SEQ ID NO: 9, 19, 29, 39, 49, 59, 69, 79, 89, 99, 118, 128, 138, 148, 158, 168. In some embodiments, the alpha chain of the TCR comprises an amino acid sequence having at least 90%, 95%, or 100% identity to the amino acid sequence of SEQ ID NO: 5, 15, 25, 35, 45, 55, 65, 75, 85, 95, 114, 124, 134, 144, 154, 164, 260, 262, 264, 266, 268, 270, 272, 274, 276, 278, 280, 282, 284, 286, 288, 290. In some embodiments, the beta chain of the TCR comprises an amino acid sequence having at least 90%, 95%, or 100% identity to the amino acid sequence of SEQ ID NO: 10, 20, 30, 40, 50, 60, 70, 80, 90, 100, 119, 129, 139, 149, 159, 169, 261, 263, 265, 267, 269, 271, 273, 275, 277, 279, 281, 283, 285, 287, 289, 291.

In some embodiments, the alpha chain variable region of the TCR comprises an amino acid sequence having at least 90%, 95%, or 100% identity to the amino acid sequence of SEQ ID NO: 4; and the beta chain variable region of the TCR comprises an amino acid sequence having at least 90%, 95%, or 100% identity to the amino acid sequence of SEQ ID NO: 9. In some embodiments, the alpha chain of the TCR comprises an amino acid sequence having at least 90%, 95%, or 100% identity to the amino acid sequence of SEQ ID NO: 5; and the beta chain of the TCR comprises an amino acid sequence having at least 90%, 95%, or 100% identity to the amino acid sequence of SEQ ID NO: 10. In some embodiments, the alpha chain of the TCR comprises an amino acid sequence having at least 90%, 95%, or 100% identity to the amino acid sequence of SEQ ID NO: 260; and the beta chain of the TCR comprises an amino acid sequence having at least 90%, 95%, or 100% identity to the amino acid sequence of SEQ ID NO: 261.

In some embodiments, the alpha chain variable region of the TCR comprises an amino acid sequence having at least 90%, 95%, or 100% identity to the amino acid sequence of SEQ ID NO: 14; and the beta chain variable region of the TCR comprises an amino acid sequence having at least 90%, 95%, or 100% identity to the amino acid sequence of SEQ ID NO: 19. In some embodiments, the alpha chain of the TCR comprises an amino acid sequence having at least 90%, 95%, or 100% identity to the amino acid sequence of SEQ ID NO: 15; and the beta chain of the TCR comprises an amino acid sequence having at least 90%, 95%, or 100% identity to the amino acid sequence of SEQ ID NO: 20. In some embodiments, the alpha chain of the TCR comprises an amino acid sequence having at least 90%, 95%, or 100% identity to the amino acid sequence of SEQ ID NO: 262; and the beta chain of the TCR comprises an amino acid sequence having at least 90%, 95%, or 100% identity to the amino acid sequence of SEQ ID NO: 263.

In some embodiments, the alpha chain variable region of the TCR comprises an amino acid sequence having at least 90%, 95%, or 100% identity to the amino acid sequence of SEQ ID NO: 24; and the beta chain variable region of the TCR comprises an amino acid sequence having at least 90%, 95%, or 100% identity to the amino acid sequence of SEQ ID NO: 29. In some embodiments, the alpha chain of the TCR comprises an amino acid sequence having at least 90%, 95%, or 100% identity to the amino acid sequence of SEQ ID NO: 25; and the beta chain of the TCR comprises an amino acid sequence having at least 90%, 95%, or 100% identity to the amino acid sequence of SEQ ID NO: 30. In some embodiments, the alpha chain of the TCR comprises an amino acid sequence having at least 90%, 95%, or 100% identity to the amino acid sequence of SEQ ID NO: 264; and the beta chain of the TCR comprises an amino acid sequence having at least 90%, 95%, or 100% identity to the amino acid sequence of SEQ ID NO: 265.

In some embodiments, the alpha chain variable region of the TCR comprises an amino acid sequence having at least 90%, 95%, or 100% identity to the amino acid sequence of SEQ ID NO: 34; and the beta chain variable region of the TCR comprises an amino acid sequence having at least 90%, 95%, or 100% identity to the amino acid sequence of SEQ ID NO: 39. In some embodiments, the alpha chain of the TCR comprises an amino acid sequence having at least 90%, 95%, or 100% identity to the amino acid sequence of SEQ ID NO: 35; and the beta chain of the TCR comprises an amino acid sequence having at least 90%, 95%, or 100% identity to the amino acid sequence of SEQ ID NO: 40. In some embodiments, the alpha chain of the TCR comprises an amino acid sequence having at least 90%, 95%, or 100% identity to the amino acid sequence of SEQ ID NO: 266; and the beta chain of the TCR comprises an amino acid sequence having at least 90%, 95%, or 100% identity to the amino acid sequence of SEQ ID NO: 267.

In some embodiments, the alpha chain variable region of the TCR comprises an amino acid sequence having at least 90%, 95%, or 100% identity to the amino acid sequence of SEQ ID NO: 44; and the beta chain variable region of the TCR comprises an amino acid sequence having at least 90%, 95%, or 100% identity to the amino acid sequence of SEQ ID NO: 49. In some embodiments, the alpha chain of the TCR comprises an amino acid sequence having at least 90%, 95%, or 100% identity to the amino acid sequence of SEQ ID NO: 45; and the beta chain of the TCR comprises an amino acid sequence having at least 90%, 95%, or 100% identity to the amino acid sequence of SEQ ID NO: 50. In some embodiments, the alpha chain of the TCR comprises an amino acid sequence having at least 90%, 95%, or 100% identity to the amino acid sequence of SEQ ID NO: 268; and the beta chain of the TCR comprises an amino acid sequence having at least 90%, 95%, or 100% identity to the amino acid sequence of SEQ ID NO: 269.

In some embodiments, the alpha chain variable region of the TCR comprises an amino acid sequence having at least 90%, 95%, or 100% identity to the amino acid sequence of SEQ ID NO: 54; and the beta chain variable region of the TCR comprises an amino acid sequence having at least 90%, 95%, or 100% identity to the amino acid sequence of SEQ ID NO: 59. In some embodiments, the alpha chain of the TCR comprises an amino acid sequence having at least 90%, 95%, or 100% identity to the amino acid sequence of SEQ ID NO: 55; and the beta chain of the TCR comprises an amino acid sequence having at least 90%, 95%, or 100% identity to the amino acid sequence of SEQ ID NO: 60. In some embodiments, the alpha chain of the TCR comprises an amino acid sequence having at least 90%, 95%, or 100% identity to the amino acid sequence of SEQ ID NO: 270; and the beta chain of the TCR comprises an amino acid sequence having at least 90%, 95%, or 100% identity to the amino acid sequence of SEQ ID NO: 271.

In some embodiments, the alpha chain variable region of the TCR comprises an amino acid sequence having at least 90%, 95%, or 100% identity to the amino acid sequence of SEQ ID NO: 64; and the beta chain variable region of the TCR comprises an amino acid sequence having at least 90%, 95%, or 100% identity to the amino acid sequence of SEQ ID NO: 69. In some embodiments, the alpha chain of the TCR comprises an amino acid sequence having at least 90%, 95%, or 100% identity to the amino acid sequence of SEQ ID NO: 65; and the beta chain of the TCR comprises an amino acid sequence having at least 90%, 95%, or 100% identity to the amino acid sequence of SEQ ID NO: 70. In some embodiments, the alpha chain of the TCR comprises an amino acid sequence having at least 90%, 95%, or 100% identity to the amino acid sequence of SEQ ID NO: 272; and the beta chain of the TCR comprises an amino acid sequence having at least 90%, 95%, or 100% identity to the amino acid sequence of SEQ ID NO: 273.

In some embodiments, the alpha chain variable region of the TCR comprises an amino acid sequence having at least 90%, 95%, or 100% identity to the amino acid sequence of SEQ ID NO: 74; and the beta chain variable region of the TCR comprises an amino acid sequence having at least 90%, 95%, or 100% identity to the amino acid sequence of SEQ ID NO: 79. In some embodiments, the alpha chain of the TCR comprises an amino acid sequence having at least 90%, 95%, or 100% identity to the amino acid sequence of SEQ ID NO: 75; and the beta chain of the TCR comprises an amino acid sequence having at least 90%, 95%, or 100% identity to the amino acid sequence of SEQ ID NO: 80. In some embodiments, the alpha chain of the TCR comprises an amino acid sequence having at least 90%, 95%, or 100% identity to the amino acid sequence of SEQ ID NO: 274; and the beta chain of the TCR comprises an amino acid sequence having at least 90%, 95%, or 100% identity to the amino acid sequence of SEQ ID NO: 275.

In some embodiments, the alpha chain variable region of the TCR comprises an amino acid sequence having at least 90%, 95%, or 100% identity to the amino acid sequence of SEQ ID NO: 84; and the beta chain variable region of the TCR comprises an amino acid sequence having at least 90%, 95%, or 100% identity to the amino acid sequence of SEQ ID NO: 89. In some embodiments, the alpha chain of the TCR comprises an amino acid sequence having at least 90%, 95%, or 100% identity to the amino acid sequence of SEQ ID NO: 85; and the beta chain of the TCR comprises an amino acid sequence having at least 90%, 95%, or 100% identity to the amino acid sequence of SEQ ID NO: 90. In some embodiments, the alpha chain of the TCR comprises an amino acid sequence having at least 90%, 95%, or 100% identity to the amino acid sequence of SEQ ID NO: 276; and the beta chain of the TCR comprises an amino acid sequence having at least 90%, 95%, or 100% identity to the amino acid sequence of SEQ ID NO: 277.

In some embodiments, the alpha chain variable region of the TCR comprises an amino acid sequence having at least 90%, 95%, or 100% identity to the amino acid sequence of SEQ ID NO: 94; and the beta chain variable region of the TCR comprises an amino acid sequence having at least 90%, 95%, or 100% identity to the amino acid sequence of SEQ ID NO: 99. In some embodiments, the alpha chain of the TCR comprises an amino acid sequence having at least 90%, 95%, or 100% identity to the amino acid sequence of SEQ ID NO: 95; and the beta chain of the TCR comprises an amino acid sequence having at least 90%, 95%, or 100% identity to the amino acid sequence of SEQ ID NO: 100. In some embodiments, the alpha chain of the TCR comprises an amino acid sequence having at least 90%, 95%, or 100% identity to the amino acid sequence of SEQ ID NO: 278; and the beta chain of the TCR comprises an amino acid sequence having at least 90%, 95%, or 100% identity to the amino acid sequence of SEQ ID NO: 279.

In some embodiments, the alpha chain variable region of the TCR comprises an amino acid sequence having at least 90%, 95%, or 100% identity to the amino acid sequence of SEQ ID NO: 113; and the beta chain variable region of the TCR comprises an amino acid sequence having at least 90%, 95%, or 100% identity to the amino acid sequence of SEQ ID NO: 118. In some embodiments, the alpha chain of the TCR comprises an amino acid sequence having at least 90%, 95%, or 100% identity to the amino acid sequence of SEQ ID NO: 114; and the beta chain of the TCR comprises an amino acid sequence having at least 90%, 95%, or 100% identity to the amino acid sequence of SEQ ID NO: 119. In some embodiments, the alpha chain of the TCR comprises an amino acid sequence having at least 90%, 95%, or 100% identity to the amino acid sequence of SEQ ID NO: 280; and the beta chain of the TCR comprises an amino acid sequence having at least 90%, 95%, or 100% identity to the amino acid sequence of SEQ ID NO: 281.

In some embodiments, the alpha chain variable region of the TCR comprises an amino acid sequence having at least 90%, 95%, or 100% identity to the amino acid sequence of SEQ ID NO: 123; and the beta chain variable region of the TCR comprises an amino acid sequence having at least 90%, 95%, or 100% identity to the amino acid sequence of SEQ ID NO: 128. In some embodiments, the alpha chain of the TCR comprises an amino acid sequence having at least 90%, 95%, or 100% identity to the amino acid sequence of SEQ ID NO: 124; and the beta chain of the TCR comprises an amino acid sequence having at least 90%, 95%, or 100% identity to the amino acid sequence of SEQ ID NO: 129. In some embodiments, the alpha chain of the TCR comprises an amino acid sequence having at least 90%, 95%, or 100% identity to the amino acid sequence of SEQ ID NO: 282; and the beta chain of the TCR comprises an amino acid sequence having at least 90%, 95%, or 100% identity to the amino acid sequence of SEQ ID NO: 283.

In some embodiments, the alpha chain variable region of the TCR comprises an amino acid sequence having at least 90%, 95%, or 100% identity to the amino acid sequence of SEQ ID NO: 133; and the beta chain variable region of the TCR comprises an amino acid sequence having at least 90%, 95%, or 100% identity to the amino acid sequence of SEQ ID NO: 138. In some embodiments, the alpha chain of the TCR comprises an amino acid sequence having at least 90%, 95%, or 100% identity to the amino acid sequence of SEQ ID NO: 134; and the beta chain of the TCR comprises an amino acid sequence having at least 90%, 95%, or 100% identity to the amino acid sequence of SEQ ID NO: 139. In some embodiments, the alpha chain of the TCR comprises an amino acid sequence having at least 90%, 95%, or 100% identity to the amino acid sequence of SEQ ID NO: 284; and the beta chain of the TCR comprises an amino acid sequence having at least 90%, 95%, or 100% identity to the amino acid sequence of SEQ ID NO: 285.

In some embodiments, the alpha chain variable region of the TCR comprises an amino acid sequence having at least 90%, 95%, or 100% identity to the amino acid sequence of SEQ ID NO: 143; and the beta chain variable region of the TCR comprises an amino acid sequence having at least 90%, 95%, or 100% identity to the amino acid sequence of SEQ ID NO: 148. In some embodiments, the alpha chain of the TCR comprises an amino acid sequence having at least 90%, 95%, or 100% identity to the amino acid sequence of SEQ ID NO: 144; and the beta chain of the TCR comprises an amino acid sequence having at least 90%, 95%, or 100% identity to the amino acid sequence of SEQ ID NO: 149. In some embodiments, the alpha chain of the TCR comprises an amino acid sequence having at least 90%, 95%, or 100% identity to the amino acid sequence of SEQ ID NO: 286; and the beta chain of the TCR comprises an amino acid sequence having at least 90%, 95%, or 100% identity to the amino acid sequence of SEQ ID NO: 287.

In some embodiments, the alpha chain variable region of the TCR comprises an amino acid sequence having at least 90%, 95%, or 100% identity to the amino acid sequence of SEQ ID NO: 153; and the beta chain variable region of the TCR comprises an amino acid sequence having at least 90%, 95%, or 100% identity to the amino acid sequence of SEQ ID NO: 158. In some embodiments, the alpha chain of the TCR comprises an amino acid sequence having at least 90%, 95%, or 100% identity to the amino acid sequence of SEQ ID NO: 154; and the beta chain of the TCR comprises an amino acid sequence having at least 90%, 95%, or 100% identity to the amino acid sequence of SEQ ID NO: 159. In some embodiments, the alpha chain of the TCR comprises an amino acid sequence having at least 90%, 95%, or 100% identity to the amino acid sequence of SEQ ID NO: 288; and the beta chain of the TCR comprises an amino acid sequence having at least 90%, 95%, or 100% identity to the amino acid sequence of SEQ ID NO: 289.

In some embodiments, the alpha chain variable region of the TCR comprises an amino acid sequence having at least 90%, 95%, or 100% identity to the amino acid sequence of SEQ ID NO: 163; and the beta chain variable region of the TCR comprises an amino acid sequence having at least 90%, 95%, or 100% identity to the amino acid sequence of SEQ ID NO: 168. In some embodiments, the alpha chain of the TCR comprises an amino acid sequence having at least 90%, 95%, or 100% identity to the amino acid sequence of SEQ ID NO: 164; and the beta chain of the TCR comprises an amino acid sequence having at least 90%, 95%, or 100% identity to the amino acid sequence of SEQ ID NO: 169. In some embodiments, the alpha chain of the TCR comprises an amino acid sequence having at least 90%, 95%, or 100% identity to the amino acid sequence of SEQ ID NO: 290; and the beta chain of the TCR comprises an amino acid sequence having at least 90%, 95%, or 100% identity to the amino acid sequence of SEQ ID NO: 291. In some embodiments, a TCR comprises the amino acid sequences of SEQ ID NOs: 1-3 (e.g., TCRa CDRs 1-3), an amino acid sequence having at least 90% identity to the amino acid sequence of SEQ ID NO: 4 (e.g., TCRa variable domain), the amino acid sequences of SEQ ID NOs: 6-8 (e.g., TCRb CDRs 1-3), and an amino acid sequence having at least 90% identity to the amino acid sequence of SEQ ID NO: 9 (e.g., TCRb variable domain). In some embodiments, a TCR comprises the amino acid sequences of SEQ ID NOs: 1-3 (e.g., TCRa CDRs 1-3), an amino acid sequence having at least 90% identity to the amino acid sequence of SEQ ID NO: 5 (e.g., TCRa full length protein), the amino acid sequences of SEQ ID NOs: 6-8 (e.g., TCRb CDRs 1-3), and an amino acid sequence having at least 90% identity to the amino acid sequence of SEQ ID NO: 10 (e.g., TCRb full length protein). In some embodiments, a TCR comprises the amino acid sequences of SEQ ID NOs: 1-3 (e.g., TCRa CDRs 1-3), an amino acid sequence having at least 90% identity to the amino acid sequence of SEQ ID NO: 260 (e.g., TCRa full length protein), the amino acid sequences of SEQ ID NOs: 6-8 (e.g., TCRb CDRs 1-3), and an amino acid sequence having at least 90% identity to the amino acid sequence of SEQ ID NO: 261 (e.g., TCRb full length protein).

In some embodiments, a TCR comprises the amino acid sequences of SEQ ID NOs: 1-3 (e.g., TCRa CDRs 1-3), an amino acid sequence having at least 95% identity to the amino acid sequence of SEQ ID NO: 4 (e.g., TCRa variable domain), the amino acid sequences of SEQ ID NOs: 6-8 (e.g., TCRb CDRs 1-3), and an amino acid sequence having at least 95% identity to the amino acid sequence of SEQ ID NO: 9 (e.g., TCRb variable domain). In some embodiments, a TCR comprises the amino acid sequences of SEQ ID NOs: 1-3 (e.g., TCRa CDRs 1-3), an amino acid sequence having at least 95% identity to the amino acid sequence of SEQ ID NO: 5 (e.g., TCRa full length protein), the amino acid sequences of SEQ ID NOS: 6-8 (e.g., TCRb CDRs 1-3), and an amino acid sequence having at least 95% identity to the amino acid sequence of SEQ ID NO: 10 (e.g., TCRb full length protein). In some embodiments, a TCR comprises the amino acid sequences of SEQ ID NOs: 1-3 (e.g., TCRa CDRs 1-3), an amino acid sequence having at least 95% identity to the amino acid sequence of SEQ ID NO: 260 (e.g., TCRa full length protein), the amino acid sequences of SEQ ID NOs: 6-8 (e.g., TCRb CDRs 1-3), and an amino acid sequence having at least 95% identity to the amino acid sequence of SEQ ID NO: 261 (e.g., TCRb full length protein).

In some embodiments, a TCR comprises the amino acid sequences of SEQ ID NOs: 1-3 (e.g., TCRa CDRs 1-3), an amino acid sequence having at least 98% identity to the amino acid sequence of SEQ ID NO: 4 (e.g., TCRa variable domain), the amino acid sequences of SEQ ID NOs: 6-8 (e.g., TCRb CDRs 1-3), and an amino acid sequence having at least 98% identity to the amino acid sequence of SEQ ID NO: 9 (e.g., TCRb variable domain). In some embodiments, a TCR comprises the amino acid sequences of SEQ ID NOs: 1-3 (e.g., TCRa CDRs 1-3), an amino acid sequence having at least 98% identity to the amino acid sequence of SEQ ID NO: 5 (e.g., TCRa full length protein), the amino acid sequences of SEQ ID NOs: 6-8 (e.g., TCRb CDRs 1-3), and an amino acid sequence having at least 98% identity to the amino acid sequence of SEQ ID NO: 10 (e.g., TCRb full length protein). In some embodiments, a TCR comprises the amino acid sequences of SEQ ID NOs: 1-3 (e.g., TCRa CDRs 1-3), an amino acid sequence having at least 98% identity to the amino acid sequence of SEQ ID NO: 260 (e.g., TCRa full length protein), the amino acid sequences of SEQ ID NOs: 6-8 (e.g., TCRb CDRs 1-3), and an amino acid sequence having at least 98% identity to the amino acid sequence of SEQ ID NO: 261 (e.g., TCRb full length protein).

In some embodiments, a TCR comprises the amino acid sequences of SEQ ID NOs: 11-13 (e.g., TCRa CDRs 1-3), an amino acid sequence having at least 90% identity to the amino acid sequence of SEQ ID NO: 14 (e.g., TCRa variable domain), the amino acid sequences of SEQ ID NOs: 16-18 (e.g., TCRb CDRs 1-3), and an amino acid sequence having at least 90% identity to the amino acid sequence of SEQ ID NO: 19 (e.g., TCRb variable domain). In some embodiments, a TCR comprises the amino acid sequences of SEQ ID NOs: 11-13 (e.g., TCRa CDRs 1-3), an amino acid sequence having at least 90% identity to the amino acid sequence of SEQ ID NO: 15 (e.g., TCRa full length protein), the amino acid sequences of SEQ ID NOs: 16-18 (e.g., TCRb CDRs 1-3), and an amino acid sequence having at least 90% identity to the amino acid sequence of SEQ ID NO: 20 (e.g., TCRb full length protein). In some embodiments, a TCR comprises the amino acid sequences of SEQ ID NOs: 11-13 (e.g., TCRa CDRs 1-3), an amino acid sequence having at least 90% identity to the amino acid sequence of SEQ ID NO: 262 (e.g., TCRa full length protein), the amino acid sequences of SEQ ID NOs: 16-18 (e.g., TCRb CDRs 1-3), and an amino acid sequence having at least 90% identity to the amino acid sequence of SEQ ID NO: 263 (e.g., TCRb full length protein).

In some embodiments, a TCR comprises the amino acid sequences of SEQ ID NOs: 11-13 (e.g., TCRa CDRs 1-3), an amino acid sequence having at least 95% identity to the amino acid sequence of SEQ ID NO: 14 (e.g., TCRa variable domain), the amino acid sequences of SEQ ID NOs: 16-18 (e.g., TCRb CDRs 1-3), and an amino acid sequence having at least 95% identity to the amino acid sequence of SEQ ID NO: 19 (e.g., TCRb variable domain). In some embodiments, a TCR comprises the amino acid sequences of SEQ ID NOs: 11-13 (e.g., TCRa CDRs 1-3), an amino acid sequence having at least 95% identity to the amino acid sequence of SEQ ID NO: 15 (e.g., TCRa full length protein), the amino acid sequences of SEQ ID NOs: 16-18 (e.g., TCRb CDRs 1-3), and an amino acid sequence having at least 95% identity to the amino acid sequence of SEQ ID NO: 20 (e.g., TCRb full length protein). In some embodiments, a TCR comprises the amino acid sequences of SEQ ID NOs: 11-13 (e.g., TCRa CDRs 1-3), an amino acid sequence having at least 95% identity to the amino acid sequence of SEQ ID NO: 262 (e.g., TCRa full length protein), the amino acid sequences of SEQ ID NOs: 16-18 (e.g., TCRb CDRs 1-3), and an amino acid sequence having at least 95% identity to the amino acid sequence of SEQ ID NO: 263 (e.g., TCRb full length protein).

In some embodiments, a TCR comprises the amino acid sequences of SEQ ID NOs: 11-13 (e.g., TCRa CDRs 1-3), an amino acid sequence having at least 98% identity to the amino acid sequence of SEQ ID NO: 14 (e.g., TCRa variable domain), the amino acid sequences of SEQ ID NOs: 16-18 (e.g., TCRb CDRs 1-3), and an amino acid sequence having at least 98% identity to the amino acid sequence of SEQ ID NO: 19 (e.g., TCRb variable domain). In some embodiments, a TCR comprises the amino acid sequences of SEQ ID NOs: 11-13 (e.g., TCRa CDRs 1-3), an amino acid sequence having at least 98% identity to the amino acid sequence of SEQ ID NO: 15 (e.g., TCRa full length protein), the amino acid sequences of SEQ ID NOs: 16-18 (e.g., TCRb CDRs 1-3), and an amino acid sequence having at least 98% identity to the amino acid sequence of SEQ ID NO: 20 (e.g., TCRb full length protein). In some embodiments, a TCR comprises the amino acid sequences of SEQ ID NOs: 11-13 (e.g., TCRa CDRs 1-3), an amino acid sequence having at least 98% identity to the amino acid sequence of SEQ ID NO: 262 (e.g., TCRa full length protein), the amino acid sequences of SEQ ID NOs: 16-18 (e.g., TCRb CDRs 1-3), and an amino acid sequence having at least 98% identity to the amino acid sequence of SEQ ID NO: 263 (e.g., TCRb full length protein).

In some embodiments, a TCR comprises the amino acid sequences of SEQ ID NOs: 21-23 (e.g., TCRa CDRs 1-3), an amino acid sequence having at least 90% identity to the amino acid sequence of SEQ ID NO: 24 (e.g., TCRa variable domain), the amino acid sequences of SEQ ID NOs: 26-28 (e.g., TCRb CDRs 1-3), and an amino acid sequence having at least 90% identity to the amino acid sequence of SEQ ID NO: 29 (e.g., TCRb variable domain). In some embodiments, a TCR comprises the amino acid sequences of SEQ ID NOs: 21-23 (e.g., TCRa CDRs 1-3), an amino acid sequence having at least 90% identity to the amino acid sequence of SEQ ID NO: 25 (e.g., TCRa full length protein), the amino acid sequences of SEQ ID NOs: 26-28 (e.g., TCRb CDRs 1-3), and an amino acid sequence having at least 90% identity to the amino acid sequence of SEQ ID NO: 30 (e.g., TCRb full length protein). In some embodiments, a TCR comprises the amino acid sequences of SEQ ID NOs: 21-23 (e.g., TCRa CDRs 1-3), an amino acid sequence having at least 90% identity to the amino acid sequence of SEQ ID NO: 264 (e.g., TCRa full length protein), the amino acid sequences of SEQ ID NOs: 26-28 (e.g., TCRb CDRs 1-3), and an amino acid sequence having at least 90% identity to the amino acid sequence of SEQ ID NO: 265 (e.g., TCRb full length protein).

In some embodiments, a TCR comprises the amino acid sequences of SEQ ID NOs: 21-23 (e.g., TCRa CDRs 1-3), an amino acid sequence having at least 95% identity to the amino acid sequence of SEQ ID NO: 24 (e.g., TCRa variable domain), the amino acid sequences of SEQ ID NOs: 26-28 (e.g., TCRb CDRs 1-3), and an amino acid sequence having at least 95% identity to the amino acid sequence of SEQ ID NO: 29 (e.g., TCRb variable domain). In some embodiments, a TCR comprises the amino acid sequences of SEQ ID NOs: 21-23 (e.g., TCRa CDRs 1-3), an amino acid sequence having at least 95% identity to the amino acid sequence of SEQ ID NO: 25 (e.g., TCRa full length protein), the amino acid sequences of SEQ ID NOs: 26-28 (e.g., TCRb CDRs 1-3), and an amino acid sequence having at least 95% identity to the amino acid sequence of SEQ ID NO: 30 (e.g., TCRb full length protein). In some embodiments, a TCR comprises the amino acid sequences of SEQ ID NOs: 21-23 (e.g., TCRa CDRs 1-3), an amino acid sequence having at least 95% identity to the amino acid sequence of SEQ ID NO: 264 (e.g., TCRa full length protein), the amino acid sequences of SEQ ID NOs: 26-28 (e.g., TCRb CDRs 1-3), and an amino acid sequence having at least 95% identity to the amino acid sequence of SEQ ID NO: 265 (e.g., TCRb full length protein).

In some embodiments, a TCR comprises the amino acid sequences of SEQ ID NOs: 21-23 (e.g., TCRa CDRs 1-3), an amino acid sequence having at least 98% identity to the amino acid sequence of SEQ ID NO: 24 (e.g., TCRa variable domain), the amino acid sequences of SEQ ID NOs: 26-28 (e.g., TCRb CDRs 1-3), and an amino acid sequence having at least 98% identity to the amino acid sequence of SEQ ID NO: 29 (e.g., TCRb variable domain). In some embodiments, a TCR comprises the amino acid sequences of SEQ ID NOs: 21-23 (e.g., TCRa CDRs 1-3), an amino acid sequence having at least 98% identity to the amino acid sequence of SEQ ID NO: 25 (e.g., TCRa full length protein), the amino acid sequences of SEQ ID NOs: 26-28 (e.g., TCRb CDRs 1-3), and an amino acid sequence having at least 98% identity to the amino acid sequence of SEQ ID NO: 30 (e.g., TCRb full length protein). In some embodiments, a TCR comprises the amino acid sequences of SEQ ID NOs: 21-23 (e.g., TCRa CDRs 1-3), an amino acid sequence having at least 98% identity to the amino acid sequence of SEQ ID NO: 264 (e.g., TCRa full length protein), the amino acid sequences of SEQ ID NOs: 26-28 (e.g., TCRb CDRs 1-3), and an amino acid sequence having at least 98% identity to the amino acid sequence of SEQ ID NO: 265 (e.g., TCRb full length protein).

In some embodiments, a TCR comprises the amino acid sequences of SEQ ID NOs: 31-33 (e.g., TCRa CDRs 1-3), an amino acid sequence having at least 90% identity to the amino acid sequence of SEQ ID NO: 34 (e.g., TCRa variable domain), the amino acid sequences of SEQ ID NOs: 36-38 (e.g., TCRb CDRs 1-3), and an amino acid sequence having at least 90% identity to the amino acid sequence of SEQ ID NO: 39 (e.g., TCRb variable domain). In some embodiments, a TCR comprises the amino acid sequences of SEQ ID NOs: 31-33 (e.g., TCRa CDRs 1-3), an amino acid sequence having at least 90% identity to the amino acid sequence of SEQ ID NO: 35 (e.g., TCRa full length protein), the amino acid sequences of SEQ ID NOs: 36-38 (e.g., TCRb CDRs 1-3), and an amino acid sequence having at least 90% identity to the amino acid sequence of SEQ ID NO: 40 (e.g., TCRb full length protein). In some embodiments, a TCR comprises the amino acid sequences of SEQ ID NOs: 31-33 (e.g., TCRa CDRs 1-3), an amino acid sequence having at least 90% identity to the amino acid sequence of SEQ ID NO: 266 (e.g., TCRa full length protein), the amino acid sequences of SEQ ID NOs: 36-38 (e.g., TCRb CDRs 1-3), and an amino acid sequence having at least 90% identity to the amino acid sequence of SEQ ID NO: 267 (e.g., TCRb full length protein).

In some embodiments, a TCR comprises the amino acid sequences of SEQ ID NOs: 31-33 (e.g., TCRa CDRs 1-3), an amino acid sequence having at least 95% identity to the amino acid sequence of SEQ ID NO: 34 (e.g., TCRa variable domain), the amino acid sequences of SEQ ID NOs: 36-38 (e.g., TCRb CDRs 1-3), and an amino acid sequence having at least 95% identity to the amino acid sequence of SEQ ID NO: 39 (e.g., TCRb variable domain). In some embodiments, a TCR comprises the amino acid sequences of SEQ ID NOs: 31-33 (e.g., TCRa CDRs 1-3), an amino acid sequence having at least 95% identity to the amino acid sequence of SEQ ID NO: 35 (e.g., TCRa full length protein), the amino acid sequences of SEQ ID NOs: 36-38 (e.g., TCRb CDRs 1-3), and an amino acid sequence having at least 95% identity to the amino acid sequence of SEQ ID NO: 40 (e.g., TCRb full length protein). In some embodiments, a TCR comprises the amino acid sequences of SEQ ID NOs: 31-33 (e.g., TCRa CDRs 1-3), an amino acid sequence having at least 95% identity to the amino acid sequence of SEQ ID NO: 266 (e.g., TCRa full length protein), the amino acid sequences of SEQ ID NOs: 36-38 (e.g., TCRb CDRs 1-3), and an amino acid sequence having at least 95% identity to the amino acid sequence of SEQ ID NO: 267 (e.g., TCRb full length protein).

In some embodiments, a TCR comprises the amino acid sequences of SEQ ID NOS: 31-33 (e.g., TCRa CDRs 1-3), an amino acid sequence having at least 98% identity to the amino acid sequence of SEQ ID NO: 34 (e.g., TCRa variable domain), the amino acid sequences of SEQ ID NOs: 36-38 (e.g., TCRb CDRs 1-3), and an amino acid sequence having at least 98% identity to the amino acid sequence of SEQ ID NO: 39 (e.g., TCRb variable domain). In some embodiments, a TCR comprises the amino acid sequences of SEQ ID NOs: 31-33 (e.g., TCRa CDRs 1-3), an amino acid sequence having at least 98% identity to the amino acid sequence of SEQ ID NO: 35 (e.g., TCRa full length protein), the amino acid sequences of SEQ ID NOs: 36-38 (e.g., TCRb CDRs 1-3), and an amino acid sequence having at least 98% identity to the amino acid sequence of SEQ ID NO: 40 (e.g., TCRb full length protein). In some embodiments, a TCR comprises the amino acid sequences of SEQ ID NOs: 31-33 (e.g., TCRa CDRs 1-3), an amino acid sequence having at least 98% identity to the amino acid sequence of SEQ ID NO: 266 (e.g., TCRa full length protein), the amino acid sequences of SEQ ID NOs: 36-38 (e.g., TCRb CDRs 1-3), and an amino acid sequence having at least 98% identity to the amino acid sequence of SEQ ID NO: 267 (e.g., TCRb full length protein).

In some embodiments, a TCR comprises the amino acid sequences of SEQ ID NOS: 41-43 (e.g., TCRa CDRs 1-3), an amino acid sequence having at least 90% identity to the amino acid sequence of SEQ ID NO: 44 (e.g., TCRa variable domain), the amino acid sequences of SEQ ID NOs: 46-48 (e.g., TCRb CDRs 1-3), and an amino acid sequence having at least 90% identity to the amino acid sequence of SEQ ID NO: 49 (e.g., TCRb variable domain). In some embodiments, a TCR comprises the amino acid sequences of SEQ ID NOs: 41-43 (e.g., TCRa CDRs 1-3), an amino acid sequence having at least 90% identity to the amino acid sequence of SEQ ID NO: 45 (e.g., TCRa full length protein), the amino acid sequences of SEQ ID NOs: 41-43 (e.g., TCRb CDRs 1-3), and an amino acid sequence having at least 90% identity to the amino acid sequence of SEQ ID NO: 50 (e.g., TCRb full length protein). In some embodiments, a TCR comprises the amino acid sequences of SEQ ID NOs: 41-43 (e.g., TCRa CDRs 1-3), an amino acid sequence having at least 90% identity to the amino acid sequence of SEQ ID NO: 268 (e.g., TCRa full length protein), the amino acid sequences of SEQ ID NOs: 41-43 (e.g., TCRb CDRs 1-3), and an amino acid sequence having at least 90% identity to the amino acid sequence of SEQ ID NO: 269 (e.g., TCRb full length protein).

In some embodiments, a TCR comprises the amino acid sequences of SEQ ID NOs: 41-43 (e.g., TCRa CDRs 1-3), an amino acid sequence having at least 95% identity to the amino acid sequence of SEQ ID NO: 44 (e.g., TCRa variable domain), the amino acid sequences of SEQ ID NOs: 46-48 (e.g., TCRb CDRs 1-3), and an amino acid sequence having at least 95% identity to the amino acid sequence of SEQ ID NO: 49 (e.g., TCRb variable domain). In some embodiments, a TCR comprises the amino acid sequences of SEQ ID NOs: 41-43 (e.g., TCRa CDRs 1-3), an amino acid sequence having at least 95% identity to the amino acid sequence of SEQ ID NO: 45 (e.g., TCRa full length protein), the amino acid sequences of SEQ ID NOs: 41-43 (e.g., TCRb CDRs 1-3), and an amino acid sequence having at least 95% identity to the amino acid sequence of SEQ ID NO: 50 (e.g., TCRb full length protein). In some embodiments, a TCR comprises the amino acid sequences of SEQ ID NOs: 41-43 (e.g., TCRa CDRs 1-3), an amino acid sequence having at least 95% identity to the amino acid sequence of SEQ ID NO: 268 (e.g., TCRa full length protein), the amino acid sequences of SEQ ID NOs: 41-43 (e.g., TCRb CDRs 1-3), and an amino acid sequence having at least 95% identity to the amino acid sequence of SEQ ID NO: 269 (e.g., TCRb full length protein).

In some embodiments, a TCR comprises the amino acid sequences of SEQ ID NOs: 41-43 (e.g., TCRa CDRs 1-3), an amino acid sequence having at least 98% identity to the amino acid sequence of SEQ ID NO: 44 (e.g., TCRa variable domain), the amino acid sequences of SEQ ID NOs: 46-48 (e.g., TCRb CDRs 1-3), and an amino acid sequence having at least 98% identity to the amino acid sequence of SEQ ID NO: 49 (e.g., TCRb variable domain). In some embodiments, a TCR comprises the amino acid sequences of SEQ ID NOs: 41-43 (e.g., TCRa CDRs 1-3), an amino acid sequence having at least 98% identity to the amino acid sequence of SEQ ID NO: 45 (e.g., TCRa full length protein), the amino acid sequences of SEQ ID NOs: 41-43 (e.g., TCRb CDRs 1-3), and an amino acid sequence having at least 98% identity to the amino acid sequence of SEQ ID NO: 50 (e.g., TCRb full length protein). In some embodiments, a TCR comprises the amino acid sequences of SEQ ID NOs: 41-43 (e.g., TCRa CDRs 1-3), an amino acid sequence having at least 98% identity to the amino acid sequence of SEQ ID NO: 268 (e.g., TCRa full length protein), the amino acid sequences of SEQ ID NOs: 41-43 (e.g., TCRb CDRs 1-3), and an amino acid sequence having at least 98% identity to the amino acid sequence of SEQ ID NO: 269 (e.g., TCRb full length protein).

In some embodiments, a TCR comprises the amino acid sequences of SEQ ID NOs: 51-53 (e.g., TCRa CDRs 1-3), an amino acid sequence having at least 90% identity to the amino acid sequence of SEQ ID NO: 54 (e.g., TCRa variable domain), the amino acid sequences of SEQ ID NOs: 51-53 (e.g., TCRb CDRs 1-3), and an amino acid sequence having at least 90% identity to the amino acid sequence of SEQ ID NO: 55 (e.g., TCRb variable domain). In some embodiments, a TCR comprises the amino acid sequences of SEQ ID NOs: 51-53 (e.g., TCRa CDRs 1-3), an amino acid sequence having at least 90% identity to the amino acid sequence of SEQ ID NO: 59 (e.g., TCRa full length protein), the amino acid sequences of SEQ ID NOs: 51-53 (e.g., TCRb CDRs 1-3), and an amino acid sequence having at least 90% identity to the amino acid sequence of SEQ ID NO: 60 (e.g., TCRb full length protein). In some embodiments, a TCR comprises the amino acid sequences of SEQ ID NOs: 51-53 (e.g., TCRa CDRs 1-3), an amino acid sequence having at least 90% identity to the amino acid sequence of SEQ ID NO: 270 (e.g., TCRa full length protein), the amino acid sequences of SEQ ID NOs: 51-53 (e.g., TCRb CDRs 1-3), and an amino acid sequence having at least 90% identity to the amino acid sequence of SEQ ID NO: 271 (e.g., TCRb full length protein).

In some embodiments, a TCR comprises the amino acid sequences of SEQ ID NOs: 51-53 (e.g., TCRa CDRs 1-3), an amino acid sequence having at least 95% identity to the amino acid sequence of SEQ ID NO: 54 (e.g., TCRa variable domain), the amino acid sequences of SEQ ID NOs: 51-53 (e.g., TCRb CDRs 1-3), and an amino acid sequence having at least 95% identity to the amino acid sequence of SEQ ID NO: 55 (e.g., TCRb variable domain). In some embodiments, a TCR comprises the amino acid sequences of SEQ ID NOs: 51-53 (e.g., TCRa CDRs 1-3), an amino acid sequence having at least 95% identity to the amino acid sequence of SEQ ID NO: 59 (e.g., TCRa full length protein), the amino acid sequences of SEQ ID NOs: 51-53 (e.g., TCRb CDRs 1-3), and an amino acid sequence having at least 95% identity to the amino acid sequence of SEQ ID NO: 60 (e.g., TCRb full length protein). In some embodiments, a TCR comprises the amino acid sequences of SEQ ID NOs: 51-53 (e.g., TCRa CDRs 1-3), an amino acid sequence having at least 95% identity to the amino acid sequence of SEQ ID NO: 270 (e.g., TCRa full length protein), the amino acid sequences of SEQ ID NOs: 51-53 (e.g., TCRb CDRs 1-3), and an amino acid sequence having at least 95% identity to the amino acid sequence of SEQ ID NO: 271 (e.g., TCRb full length protein).

In some embodiments, a TCR comprises the amino acid sequences of SEQ ID NOS: 51-53 (e.g., TCRa CDRs 1-3), an amino acid sequence having at least 98% identity to the amino acid sequence of SEQ ID NO: 54 (e.g., TCRa variable domain), the amino acid sequences of SEQ ID NOs: 51-53 (e.g., TCRb CDRs 1-3), and an amino acid sequence having at least 98% identity to the amino acid sequence of SEQ ID NO: 55 (e.g., TCRb variable domain). In some embodiments, a TCR comprises the amino acid sequences of SEQ ID NOs: 51-53 (e.g., TCRa CDRs 1-3), an amino acid sequence having at least 98% identity to the amino acid sequence of SEQ ID NO: 59 (e.g., TCRa full length protein), the amino acid sequences of SEQ ID NOs: 51-53 (e.g., TCRb CDRs 1-3), and an amino acid sequence having at least 98% identity to the amino acid sequence of SEQ ID NO: 60 (e.g., TCRb full length protein). In some embodiments, a TCR comprises the amino acid sequences of SEQ ID NOs: 51-53 (e.g., TCRa CDRs 1-3), an amino acid sequence having at least 98% identity to the amino acid sequence of SEQ ID NO: 270 (e.g., TCRa full length protein), the amino acid sequences of SEQ ID NOs: 51-53 (e.g., TCRb CDRs 1-3), and an amino acid sequence having at least 98% identity to the amino acid sequence of SEQ ID NO: 271 (e.g., TCRb full length protein).

In some embodiments, a TCR comprises the amino acid sequences of SEQ ID NOs: 61-63 (e.g., TCRa CDRs 1-3), an amino acid sequence having at least 90% identity to the amino acid sequence of SEQ ID NO: 64 (e.g., TCRa variable domain), the amino acid sequences of SEQ ID NOs: 66-68 (e.g., TCRb CDRs 1-3), and an amino acid sequence having at least 90% identity to the amino acid sequence of SEQ ID NO: 69 (e.g., TCRb variable domain). In some embodiments, a TCR comprises the amino acid sequences of SEQ ID NOs: 61-63 (e.g., TCRa CDRs 1-3), an amino acid sequence having at least 90% identity to the amino acid sequence of SEQ ID NO: 65 (e.g., TCRa full length protein), the amino acid sequences of SEQ ID NOs: 61-63 (e.g., TCRb CDRs 1-3), and an amino acid sequence having at least 90% identity to the amino acid sequence of SEQ ID NO: 70 (e.g., TCRb full length protein). In some embodiments, a TCR comprises the amino acid sequences of SEQ ID NOs: 61-63 (e.g., TCRa CDRs 1-3), an amino acid sequence having at least 90% identity to the amino acid sequence of SEQ ID NO: 272 (e.g., TCRa full length protein), the amino acid sequences of SEQ ID NOs: 61-63 (e.g., TCRb CDRs 1-3), and an amino acid sequence having at least 90% identity to the amino acid sequence of SEQ ID NO: 273 (e.g., TCRb full length protein).

In some embodiments, a TCR comprises the amino acid sequences of SEQ ID NOs: 61-63 (e.g., TCRa CDRs 1-3), an amino acid sequence having at least 95% identity to the amino acid sequence of SEQ ID NO: 64 (e.g., TCRa variable domain), the amino acid sequences of SEQ ID NOs: 66-68 (e.g., TCRb CDRs 1-3), and an amino acid sequence having at least 95% identity to the amino acid sequence of SEQ ID NO: 69 (e.g., TCRb variable domain). In some embodiments, a TCR comprises the amino acid sequences of SEQ ID NOs: 61-63 (e.g., TCRa CDRs 1-3), an amino acid sequence having at least 95% identity to the amino acid sequence of SEQ ID NO: 65 (e.g., TCRa full length protein), the amino acid sequences of SEQ ID NOs: 61-63 (e.g., TCRb CDRs 1-3), and an amino acid sequence having at least 95% identity to the amino acid sequence of SEQ ID NO: 70 (e.g., TCRb full length protein). In some embodiments, a TCR comprises the amino acid sequences of SEQ ID NOs: 61-63 (e.g., TCRa CDRs 1-3), an amino acid sequence having at least 95% identity to the amino acid sequence of SEQ ID NO: 272 (e.g., TCRa full length protein), the amino acid sequences of SEQ ID NOs: 61-63 (e.g., TCRb CDRs 1-3), and an amino acid sequence having at least 95% identity to the amino acid sequence of SEQ ID NO: 273 (e.g., TCRb full length protein).

In some embodiments, a TCR comprises the amino acid sequences of SEQ ID NOs: 61-63 (e.g., TCRa CDRs 1-3), an amino acid sequence having at least 98% identity to the amino acid sequence of SEQ ID NO: 64 (e.g., TCRa variable domain), the amino acid sequences of SEQ ID NOs: 66-68 (e.g., TCRb CDRs 1-3), and an amino acid sequence having at least 98% identity to the amino acid sequence of SEQ ID NO: 69 (e.g., TCRb variable domain). In some embodiments, a TCR comprises the amino acid sequences of SEQ ID NOs: 61-63 (e.g., TCRa CDRs 1-3), an amino acid sequence having at least 98% identity to the amino acid sequence of SEQ ID NO: 65 (e.g., TCRa full length protein), the amino acid sequences of SEQ ID NOs: 61-63 (e.g., TCRb CDRs 1-3), and an amino acid sequence having at least 98% identity to the amino acid sequence of SEQ ID NO: 70 (e.g., TCRb full length protein). In some embodiments, a TCR comprises the amino acid sequences of SEQ ID NOs: 61-63 (e.g., TCRa CDRs 1-3), an amino acid sequence having at least 98% identity to the amino acid sequence of SEQ ID NO: 272 (e.g., TCRa full length protein), the amino acid sequences of SEQ ID NOs: 61-63 (e.g., TCRb CDRs 1-3), and an amino acid sequence having at least 98% identity to the amino acid sequence of SEQ ID NO: 273 (e.g., TCRb full length protein).

In some embodiments, a TCR comprises the amino acid sequences of SEQ ID NOS: 71-73 (e.g., TCRa CDRs 1-3), an amino acid sequence having at least 90% identity to the amino acid sequence of SEQ ID NO: 74 (e.g., TCRa variable domain), the amino acid sequences of SEQ ID NOs: 76-78 (e.g., TCRb CDRs 1-3), and an amino acid sequence having at least 90% identity to the amino acid sequence of SEQ ID NO: 79 (e.g., TCRb variable domain). In some embodiments, a TCR comprises the amino acid sequences of SEQ ID NOs: 71-73 (e.g., TCRa CDRs 1-3), an amino acid sequence having at least 90% identity to the amino acid sequence of SEQ ID NO: 75 (e.g., TCRa full length protein), the amino acid sequences of SEQ ID NOs: 76-78 (e.g., TCRb CDRs 1-3), and an amino acid sequence having at least 90% identity to the amino acid sequence of SEQ ID NO: 80 (e.g., TCRb full length protein). In some embodiments, a TCR comprises the amino acid sequences of SEQ ID NOs: 71-73 (e.g., TCRa CDRs 1-3), an amino acid sequence having at least 90% identity to the amino acid sequence of SEQ ID NO: 274 (e.g., TCRa full length protein), the amino acid sequences of SEQ ID NOs: 76-78 (e.g., TCRb CDRs 1-3), and an amino acid sequence having at least 90% identity to the amino acid sequence of SEQ ID NO: 275 (e.g., TCRb full length protein).

In some embodiments, a TCR comprises the amino acid sequences of SEQ ID NOs: 71-73 (e.g., TCRa CDRs 1-3), an amino acid sequence having at least 95% identity to the amino acid sequence of SEQ ID NO: 74 (e.g., TCRa variable domain), the amino acid sequences of SEQ ID NOs: 76-78 (e.g., TCRb CDRs 1-3), and an amino acid sequence having at least 95% identity to the amino acid sequence of SEQ ID NO: 79 (e.g., TCRb variable domain). In some embodiments, a TCR comprises the amino acid sequences of SEQ ID NOs: 71-73 (e.g., TCRa CDRs 1-3), an amino acid sequence having at least 95% identity to the amino acid sequence of SEQ ID NO: 75 (e.g., TCRa full length protein), the amino acid sequences of SEQ ID NOs: 76-78 (e.g., TCRb CDRs 1-3), and an amino acid sequence having at least 95% identity to the amino acid sequence of SEQ ID NO: 80 (e.g., TCRb full length protein). In some embodiments, a TCR comprises the amino acid sequences of SEQ ID NOs: 71-73 (e.g., TCRa CDRs 1-3), an amino acid sequence having at least 95% identity to the amino acid sequence of SEQ ID NO: 274 (e.g., TCRa full length protein), the amino acid sequences of SEQ ID NOs: 76-78 (e.g., TCRb CDRs 1-3), and an amino acid sequence having at least 95% identity to the amino acid sequence of SEQ ID NO: 275 (e.g., TCRb full length protein).

In some embodiments, a TCR comprises the amino acid sequences of SEQ ID NOs: 71-73 (e.g., TCRa CDRs 1-3), an amino acid sequence having at least 98% identity to the amino acid sequence of SEQ ID NO: 74 (e.g., TCRa variable domain), the amino acid sequences of SEQ ID NOs: 76-78 (e.g., TCRb CDRs 1-3), and an amino acid sequence having at least 98% identity to the amino acid sequence of SEQ ID NO: 79 (e.g., TCRb variable domain). In some embodiments, a TCR comprises the amino acid sequences of SEQ ID NOs: 71-73 (e.g., TCRa CDRs 1-3), an amino acid sequence having at least 98% identity to the amino acid sequence of SEQ ID NO: 75 (e.g., TCRa full length protein), the amino acid sequences of SEQ ID NOs: 76-78 (e.g., TCRb CDRs 1-3), and an amino acid sequence having at least 98% identity to the amino acid sequence of SEQ ID NO: 80 (e.g., TCRb full length protein). In some embodiments, a TCR comprises the amino acid sequences of SEQ ID NOs: 71-73 (e.g., TCRa CDRs 1-3), an amino acid sequence having at least 98% identity to the amino acid sequence of SEQ ID NO: 274 (e.g., TCRa full length protein), the amino acid sequences of SEQ ID NOs: 76-78 (e.g., TCRb CDRs 1-3), and an amino acid sequence having at least 98% identity to the amino acid sequence of SEQ ID NO: 275 (e.g., TCRb full length protein).

In some embodiments, a TCR comprises the amino acid sequences of SEQ ID NOs: 81-83 (e.g., TCRa CDRs 1-3), an amino acid sequence having at least 90% identity to the amino acid sequence of SEQ ID NO: 84 (e.g., TCRa variable domain), the amino acid sequences of SEQ ID NOs: 86-88 (e.g., TCRb CDRs 1-3), and an amino acid sequence having at least 90% identity to the amino acid sequence of SEQ ID NO: 89 (e.g., TCRb variable domain). In some embodiments, a TCR comprises the amino acid sequences of SEQ ID NOs: 81-83 (e.g., TCRa CDRs 1-3), an amino acid sequence having at least 90% identity to the amino acid sequence of SEQ ID NO: 85 (e.g., TCRa full length protein), the amino acid sequences of SEQ ID NOs: 86-88 (e.g., TCRb CDRs 1-3), and an amino acid sequence having at least 90% identity to the amino acid sequence of SEQ ID NO: 90 (e.g., TCRb full length protein). In some embodiments, a TCR comprises the amino acid sequences of SEQ ID NOs: 81-83 (e.g., TCRa CDRs 1-3), an amino acid sequence having at least 90% identity to the amino acid sequence of SEQ ID NO: 276 (e.g., TCRa full length protein), the amino acid sequences of SEQ ID NOs: 86-88 (e.g., TCRb CDRs 1-3), and an amino acid sequence having at least 90% identity to the amino acid sequence of SEQ ID NO: 277 (e.g., TCRb full length protein).

In some embodiments, a TCR comprises the amino acid sequences of SEQ ID NOS: 81-83 (e.g., TCRa CDRs 1-3), an amino acid sequence having at least 95% identity to the amino acid sequence of SEQ ID NO: 84 (e.g., TCRa variable domain), the amino acid sequences of SEQ ID NOs: 86-88 (e.g., TCRb CDRs 1-3), and an amino acid sequence having at least 95% identity to the amino acid sequence of SEQ ID NO: 89 (e.g., TCRb variable domain). In some embodiments, a TCR comprises the amino acid sequences of SEQ ID NOs: 81-83 (e.g., TCRa CDRs 1-3), an amino acid sequence having at least 95% identity to the amino acid sequence of SEQ ID NO: 85 (e.g., TCRa full length protein), the amino acid sequences of SEQ ID NOs: 86-88 (e.g., TCRb CDRs 1-3), and an amino acid sequence having at least 95% identity to the amino acid sequence of SEQ ID NO: 90 (e.g., TCRb full length protein). In some embodiments, a TCR comprises the amino acid sequences of SEQ ID NOs: 81-83 (e.g., TCRa CDRs 1-3), an amino acid sequence having at least 95% identity to the amino acid sequence of SEQ ID NO: 276 (e.g., TCRa full length protein), the amino acid sequences of SEQ ID NOs: 86-88 (e.g., TCRb CDRs 1-3), and an amino acid sequence having at least 95% identity to the amino acid sequence of SEQ ID NO: 277 (e.g., TCRb full length protein).

In some embodiments, a TCR comprises the amino acid sequences of SEQ ID NOs: 81-83 (e.g., TCRa CDRs 1-3), an amino acid sequence having at least 98% identity to the amino acid sequence of SEQ ID NO: 84 (e.g., TCRa variable domain), the amino acid sequences of SEQ ID NOs: 86-88 (e.g., TCRb CDRs 1-3), and an amino acid sequence having at least 98% identity to the amino acid sequence of SEQ ID NO: 89 (e.g., TCRb variable domain). In some embodiments, a TCR comprises the amino acid sequences of SEQ ID NOs: 81-83 (e.g., TCRa CDRs 1-3), an amino acid sequence having at least 98% identity to the amino acid sequence of SEQ ID NO: 85 (e.g., TCRa full length protein), the amino acid sequences of SEQ ID NOs: 86-88 (e.g., TCRb CDRs 1-3), and an amino acid sequence having at least 98% identity to the amino acid sequence of SEQ ID NO: 90 (e.g., TCRb full length protein). In some embodiments, a TCR comprises the amino acid sequences of SEQ ID NOs: 81-83 (e.g., TCRa CDRs 1-3), an amino acid sequence having at least 98% identity to the amino acid sequence of SEQ ID NO: 276 (e.g., TCRa full length protein), the amino acid sequences of SEQ ID NOs: 86-88 (e.g., TCRb CDRs 1-3), and an amino acid sequence having at least 98% identity to the amino acid sequence of SEQ ID NO: 277 (e.g., TCRb full length protein).

In some embodiments, a TCR comprises the amino acid sequences of SEQ ID NOs: 91-93 (e.g., TCRa CDRs 1-3), an amino acid sequence having at least 90% identity to the amino acid sequence of SEQ ID NO: 94 (e.g., TCRa variable domain), the amino acid sequences of SEQ ID NOs: 96-98 (e.g., TCRb CDRs 1-3), and an amino acid sequence having at least 90% identity to the amino acid sequence of SEQ ID NO: 99 (e.g., TCRb variable domain). In some embodiments, a TCR comprises the amino acid sequences of SEQ ID NOs: 91-93 (e.g., TCRa CDRs 1-3), an amino acid sequence having at least 90% identity to the amino acid sequence of SEQ ID NO: 95 (e.g., TCRa full length protein), the amino acid sequences of SEQ ID NOs: 96-98 (e.g., TCRb CDRs 1-3), and an amino acid sequence having at least 90% identity to the amino acid sequence of SEQ ID NO: 100 (e.g., TCRb full length protein). In some embodiments, a TCR comprises the amino acid sequences of SEQ ID NOs: 91-93 (e.g., TCRa CDRs 1-3), an amino acid sequence having at least 90% identity to the amino acid sequence of SEQ ID NO: 278 (e.g., TCRa full length protein), the amino acid sequences of SEQ ID NOs: 96-98 (e.g., TCRb CDRs 1-3), and an amino acid sequence having at least 90% identity to the amino acid sequence of SEQ ID NO: 279 (e.g., TCRb full length protein).

In some embodiments, a TCR comprises the amino acid sequences of SEQ ID NOS: 91-93 (e.g., TCRa CDRs 1-3), an amino acid sequence having at least 95% identity to the amino acid sequence of SEQ ID NO: 94 (e.g., TCRa variable domain), the amino acid sequences of SEQ ID NOs: 96-98 (e.g., TCRb CDRs 1-3), and an amino acid sequence having at least 95% identity to the amino acid sequence of SEQ ID NO: 99 (e.g., TCRb variable domain). In some embodiments, a TCR comprises the amino acid sequences of SEQ ID NOs: 91-93 (e.g., TCRa CDRs 1-3), an amino acid sequence having at least 95% identity to the amino acid sequence of SEQ ID NO: 95 (e.g., TCRa full length protein), the amino acid sequences of SEQ ID NOs: 96-98 (e.g., TCRb CDRs 1-3), and an amino acid sequence having at least 95% identity to the amino acid sequence of SEQ ID NO: 100 (e.g., TCRb full length protein). In some embodiments, a TCR comprises the amino acid sequences of SEQ ID NOs: 91-93 (e.g., TCRa CDRs 1-3), an amino acid sequence having at least 95% identity to the amino acid sequence of SEQ ID NO: 278 (e.g., TCRa full length protein), the amino acid sequences of SEQ ID NOs: 96-98 (e.g., TCRb CDRs 1-3), and an amino acid sequence having at least 95% identity to the amino acid sequence of SEQ ID NO: 279 (e.g., TCRb full length protein).

In some embodiments, a TCR comprises the amino acid sequences of SEQ ID NOS: 91-93 (e.g., TCRa CDRs 1-3), an amino acid sequence having at least 98% identity to the amino acid sequence of SEQ ID NO: 94 (e.g., TCRa variable domain), the amino acid sequences of SEQ ID NOs: 96-98 (e.g., TCRb CDRs 1-3), and an amino acid sequence having at least 98% identity to the amino acid sequence of SEQ ID NO: 99 (e.g., TCRb variable domain). In some embodiments, a TCR comprises the amino acid sequences of SEQ ID NOs: 91-93 (e.g., TCRa CDRs 1-3), an amino acid sequence having at least 98% identity to the amino acid sequence of SEQ ID NO: 95 (e.g., TCRa full length protein), the amino acid sequences of SEQ ID NOs: 96-98 (e.g., TCRb CDRs 1-3), and an amino acid sequence having at least 98% identity to the amino acid sequence of SEQ ID NO: 100 (e.g., TCRb full length protein). In some embodiments, a TCR comprises the amino acid sequences of SEQ ID NOs: 91-93 (e.g., TCRa CDRs 1-3), an amino acid sequence having at least 98% identity to the amino acid sequence of SEQ ID NO: 278 (e.g., TCRa full length protein), the amino acid sequences of SEQ ID NOs: 96-98 (e.g., TCRb CDRs 1-3), and an amino acid sequence having at least 98% identity to the amino acid sequence of SEQ ID NO: 279 (e.g., TCRb full length protein).

In some embodiments, a TCR comprises the amino acid sequences of SEQ ID NOs: 110-112 (e.g., TCRa CDRs 1-3), an amino acid sequence having at least 90% identity to the amino acid sequence of SEQ ID NO: 113 (e.g., TCRa variable domain), the amino acid sequences of SEQ ID NOs: 115-117 (e.g., TCRb CDRs 1-3), and an amino acid sequence having at least 90% identity to the amino acid sequence of SEQ ID NO: 118 (e.g., TCRb variable domain). In some embodiments, a TCR comprises the amino acid sequences of SEQ ID NOs: 110-112 (e.g., TCRa CDRs 1-3), an amino acid sequence having at least 90% identity to the amino acid sequence of SEQ ID NO: 114 (e.g., TCRa full length protein), the amino acid sequences of SEQ ID NOs: 115-117 (e.g., TCRb CDRs 1-3), and an amino acid sequence having at least 90% identity to the amino acid sequence of SEQ ID NO: 119 (e.g., TCRb full length protein). In some embodiments, a TCR comprises the amino acid sequences of SEQ ID NOs: 110-112 (e.g., TCRa CDRs 1-3), an amino acid sequence having at least 90% identity to the amino acid sequence of SEQ ID NO: 280 (e.g., TCRa full length protein), the amino acid sequences of SEQ ID NOs: 115-117 (e.g., TCRb CDRs 1-3), and an amino acid sequence having at least 90% identity to the amino acid sequence of SEQ ID NO: 281 (e.g., TCRb full length protein).

In some embodiments, a TCR comprises the amino acid sequences of SEQ ID NOs: 110-112 (e.g., TCRa CDRs 1-3), an amino acid sequence having at least 95% identity to the amino acid sequence of SEQ ID NO: 113 (e.g., TCRa variable domain), the amino acid sequences of SEQ ID NOs: 115-117 (e.g., TCRb CDRs 1-3), and an amino acid sequence having at least 95% identity to the amino acid sequence of SEQ ID NO: 118 (e.g., TCRb variable domain). In some embodiments, a TCR comprises the amino acid sequences of SEQ ID NOs: 110-112 (e.g., TCRa CDRs 1-3), an amino acid sequence having at least 95% identity to the amino acid sequence of SEQ ID NO: 114 (e.g., TCRa full length protein), the amino acid sequences of SEQ ID NOs: 115-117 (e.g., TCRb CDRs 1-3), and an amino acid sequence having at least 95% identity to the amino acid sequence of SEQ ID NO: 119 (e.g., TCRb full length protein). In some embodiments, a TCR comprises the amino acid sequences of SEQ ID NOs: 110-112 (e.g., TCRa CDRs 1-3), an amino acid sequence having at least 95% identity to the amino acid sequence of SEQ ID NO: 280 (e.g., TCRa full length protein), the amino acid sequences of SEQ ID NOs: 115-117 (e.g., TCRb CDRs 1-3), and an amino acid sequence having at least 95% identity to the amino acid sequence of SEQ ID NO: 281 (e.g., TCRb full length protein).

In some embodiments, a TCR comprises the amino acid sequences of SEQ ID NOs: 110-112 (e.g., TCRa CDRs 1-3), an amino acid sequence having at least 98% identity to the amino acid sequence of SEQ ID NO: 113 (e.g., TCRa variable domain), the amino acid sequences of SEQ ID NOs: 115-117 (e.g., TCRb CDRs 1-3), and an amino acid sequence having at least 98% identity to the amino acid sequence of SEQ ID NO: 118 (e.g., TCRb variable domain). In some embodiments, a TCR comprises the amino acid sequences of SEQ ID NOs: 110-112 (e.g., TCRa CDRs 1-3), an amino acid sequence having at least 98% identity to the amino acid sequence of SEQ ID NO: 114 (e.g., TCRa full length protein), the amino acid sequences of SEQ ID NOs: 115-117 (e.g., TCRb CDRs 1-3), and an amino acid sequence having at least 98% identity to the amino acid sequence of SEQ ID NO: 119 (e.g., TCRb full length protein). In some embodiments, a TCR comprises the amino acid sequences of SEQ ID NOs: 110-112 (e.g., TCRa CDRs 1-3), an amino acid sequence having at least 98% identity to the amino acid sequence of SEQ ID NO: 280 (e.g., TCRa full length protein), the amino acid sequences of SEQ ID NOs: 115-117 (e.g., TCRb CDRs 1-3), and an amino acid sequence having at least 98% identity to the amino acid sequence of SEQ ID NO: 281 (e.g., TCRb full length protein).

In some embodiments, a TCR comprises the amino acid sequences of SEQ ID NOs: 120-122 (e.g., TCRa CDRs 1-3), an amino acid sequence having at least 90% identity to the amino acid sequence of SEQ ID NO: 123 (e.g., TCRa variable domain), the amino acid sequences of SEQ ID NOs: 125-127 (e.g., TCRb CDRs 1-3), and an amino acid sequence having at least 90% identity to the amino acid sequence of SEQ ID NO: 128 (e.g., TCRb variable domain). In some embodiments, a TCR comprises the amino acid sequences of SEQ ID NOs: 120-122 (e.g., TCRa CDRs 1-3), an amino acid sequence having at least 90% identity to the amino acid sequence of SEQ ID NO: 124 (e.g., TCRa full length protein), the amino acid sequences of SEQ ID NOs: 125-127 (e.g., TCRb CDRs 1-3), and an amino acid sequence having at least 90% identity to the amino acid sequence of SEQ ID NO: 129 (e.g., TCRb full length protein). In some embodiments, a TCR comprises the amino acid sequences of SEQ ID NOs: 120-122 (e.g., TCRa CDRs 1-3), an amino acid sequence having at least 90% identity to the amino acid sequence of SEQ ID NO: 282 (e.g., TCRa full length protein), the amino acid sequences of SEQ ID NOs: 125-127 (e.g., TCRb CDRs 1-3), and an amino acid sequence having at least 90% identity to the amino acid sequence of SEQ ID NO: 283 (e.g., TCRb full length protein).

In some embodiments, a TCR comprises the amino acid sequences of SEQ ID NOs: 120-122 (e.g., TCRa CDRs 1-3), an amino acid sequence having at least 95% identity to the amino acid sequence of SEQ ID NO: 123 (e.g., TCRa variable domain), the amino acid sequences of SEQ ID NOs: 125-127 (e.g., TCRb CDRs 1-3), and an amino acid sequence having at least 95% identity to the amino acid sequence of SEQ ID NO: 128 (e.g., TCRb variable domain). In some embodiments, a TCR comprises the amino acid sequences of SEQ ID NOs: 120-122 (e.g., TCRa CDRs 1-3), an amino acid sequence having at least 95% identity to the amino acid sequence of SEQ ID NO: 124 (e.g., TCRa full length protein), the amino acid sequences of SEQ ID NOs: 125-127 (e.g., TCRb CDRs 1-3), and an amino acid sequence having at least 95% identity to the amino acid sequence of SEQ ID NO: 129 (e.g., TCRb full length protein). In some embodiments, a TCR comprises the amino acid sequences of SEQ ID NOs: 120-122 (e.g., TCRa CDRs 1-3), an amino acid sequence having at least 95% identity to the amino acid sequence of SEQ ID NO: 282 (e.g., TCRa full length protein), the amino acid sequences of SEQ ID NOs: 125-127 (e.g., TCRb CDRs 1-3), and an amino acid sequence having at least 95% identity to the amino acid sequence of SEQ ID NO: 283 (e.g., TCRb full length protein).

In some embodiments, a TCR comprises the amino acid sequences of SEQ ID NOs: 120-122 (e.g., TCRa CDRs 1-3), an amino acid sequence having at least 98% identity to the amino acid sequence of SEQ ID NO: 123 (e.g., TCRa variable domain), the amino acid sequences of SEQ ID NOs: 125-127 (e.g., TCRb CDRs 1-3), and an amino acid sequence having at least 98% identity to the amino acid sequence of SEQ ID NO: 128 (e.g., TCRb variable domain). In some embodiments, a TCR comprises the amino acid sequences of SEQ ID NOs: 120-122 (e.g., TCRa CDRs 1-3), an amino acid sequence having at least 98% identity to the amino acid sequence of SEQ ID NO: 124 (e.g., TCRa full length protein), the amino acid sequences of SEQ ID NOs: 125-127 (e.g., TCRb CDRs 1-3), and an amino acid sequence having at least 98% identity to the amino acid sequence of SEQ ID NO: 129 (e.g., TCRb full length protein). In some embodiments, a TCR comprises the amino acid sequences of SEQ ID NOs: 120-122 (e.g., TCRa CDRs 1-3), an amino acid sequence having at least 98% identity to the amino acid sequence of SEQ ID NO: 282 (e.g., TCRa full length protein), the amino acid sequences of SEQ ID NOs: 125-127 (e.g., TCRb CDRs 1-3), and an amino acid sequence having at least 98% identity to the amino acid sequence of SEQ ID NO: 283 (e.g., TCRb full length protein).

In some embodiments, a TCR comprises the amino acid sequences of SEQ ID NOs: 130-132 (e.g., TCRa CDRs 1-3), an amino acid sequence having at least 90% identity to the amino acid sequence of SEQ ID NO: 133 (e.g., TCRa variable domain), the amino acid sequences of SEQ ID NOs: 135-137 (e.g., TCRb CDRs 1-3), and an amino acid sequence having at least 90% identity to the amino acid sequence of SEQ ID NO: 138 (e.g., TCRb variable domain). In some embodiments, a TCR comprises the amino acid sequences of SEQ ID NOs: 130-132 (e.g., TCRa CDRs 1-3), an amino acid sequence having at least 90% identity to the amino acid sequence of SEQ ID NO: 134 (e.g., TCRa full length protein), the amino acid sequences of SEQ ID NOs: 135-137 (e.g., TCRb CDRs 1-3), and an amino acid sequence having at least 90% identity to the amino acid sequence of SEQ ID NO: 139 (e.g., TCRb full length protein). In some embodiments, a TCR comprises the amino acid sequences of SEQ ID NOs: 130-132 (e.g., TCRa CDRs 1-3), an amino acid sequence having at least 90% identity to the amino acid sequence of SEQ ID NO: 284 (e.g., TCRa full length protein), the amino acid sequences of SEQ ID NOs: 135-137 (e.g., TCRb CDRs 1-3), and an amino acid sequence having at least 90% identity to the amino acid sequence of SEQ ID NO: 285 (e.g., TCRb full length protein).

In some embodiments, a TCR comprises the amino acid sequences of SEQ ID NOs: 130-132 (e.g., TCRa CDRs 1-3), an amino acid sequence having at least 95% identity to the amino acid sequence of SEQ ID NO: 133 (e.g., TCRa variable domain), the amino acid sequences of SEQ ID NOs: 135-137 (e.g., TCRb CDRs 1-3), and an amino acid sequence having at least 95% identity to the amino acid sequence of SEQ ID NO: 138 (e.g., TCRb variable domain). In some embodiments, a TCR comprises the amino acid sequences of SEQ ID NOs: 130-132 (e.g., TCRa CDRs 1-3), an amino acid sequence having at least 95% identity to the amino acid sequence of SEQ ID NO: 134 (e.g., TCRa full length protein), the amino acid sequences of SEQ ID NOs: 135-137 (e.g., TCRb CDRs 1-3), and an amino acid sequence having at least 95% identity to the amino acid sequence of SEQ ID NO: 139 (e.g., TCRb full length protein). In some embodiments, a TCR comprises the amino acid sequences of SEQ ID NOs: 130-132 (e.g., TCRa CDRs 1-3), an amino acid sequence having at least 95% identity to the amino acid sequence of SEQ ID NO: 284 (e.g., TCRa full length protein), the amino acid sequences of SEQ ID NOs: 135-137 (e.g., TCRb CDRs 1-3), and an amino acid sequence having at least 95% identity to the amino acid sequence of SEQ ID NO: 285 (e.g., TCRb full length protein).

In some embodiments, a TCR comprises the amino acid sequences of SEQ ID NOs: 130-132 (e.g., TCRa CDRs 1-3), an amino acid sequence having at least 98% identity to the amino acid sequence of SEQ ID NO: 133 (e.g., TCRa variable domain), the amino acid sequences of SEQ ID NOs: 135-137 (e.g., TCRb CDRs 1-3), and an amino acid sequence having at least 98% identity to the amino acid sequence of SEQ ID NO: 138 (e.g., TCRb variable domain). In some embodiments, a TCR comprises the amino acid sequences of SEQ ID NOs: 130-132 (e.g., TCRa CDRs 1-3), an amino acid sequence having at least 98% identity to the amino acid sequence of SEQ ID NO: 134 (e.g., TCRa full length protein), the amino acid sequences of SEQ ID NOs: 135-137 (e.g., TCRb CDRs 1-3), and an amino acid sequence having at least 98% identity to the amino acid sequence of SEQ ID NO: 139 (e.g., TCRb full length protein). In some embodiments, a TCR comprises the amino acid sequences of SEQ ID NOs: 130-132 (e.g., TCRa CDRs 1-3), an amino acid sequence having at least 98% identity to the amino acid sequence of SEQ ID NO: 284 (e.g., TCRa full length protein), the amino acid sequences of SEQ ID NOs: 135-137 (e.g., TCRb CDRs 1-3), and an amino acid sequence having at least 98% identity to the amino acid sequence of SEQ ID NO: 285 (e.g., TCRb full length protein).

In some embodiments, a TCR comprises the amino acid sequences of SEQ ID NOs: 140-142 (e.g., TCRa CDRs 1-3), an amino acid sequence having at least 90% identity to the amino acid sequence of SEQ ID NO: 143 (e.g., TCRa variable domain), the amino acid sequences of SEQ ID NOs: 145-147 (e.g., TCRb CDRs 1-3), and an amino acid sequence having at least 90% identity to the amino acid sequence of SEQ ID NO: 148 (e.g., TCRb variable domain). In some embodiments, a TCR comprises the amino acid sequences of SEQ ID NOs: 140-142 (e.g., TCRa CDRs 1-3), an amino acid sequence having at least 90% identity to the amino acid sequence of SEQ ID NO: 144 (e.g., TCRa full length protein), the amino acid sequences of SEQ ID NOs: 145-147 (e.g., TCRb CDRs 1-3), and an amino acid sequence having at least 90% identity to the amino acid sequence of SEQ ID NO: 149 (e.g., TCRb full length protein). In some embodiments, a TCR comprises the amino acid sequences of SEQ ID NOs: 140-142 (e.g., TCRa CDRs 1-3), an amino acid sequence having at least 90% identity to the amino acid sequence of SEQ ID NO: 286 (e.g., TCRa full length protein), the amino acid sequences of SEQ ID NOs: 145-147 (e.g., TCRb CDRs 1-3), and an amino acid sequence having at least 90% identity to the amino acid sequence of SEQ ID NO: 287 (e.g., TCRb full length protein).

In some embodiments, a TCR comprises the amino acid sequences of SEQ ID NOs: 140-142 (e.g., TCRa CDRs 1-3), an amino acid sequence having at least 95% identity to the amino acid sequence of SEQ ID NO: 143 (e.g., TCRa variable domain), the amino acid sequences of SEQ ID NOs: 145-147 (e.g., TCRb CDRs 1-3), and an amino acid sequence having at least 95% identity to the amino acid sequence of SEQ ID NO: 148 (e.g., TCRb variable domain). In some embodiments, a TCR comprises the amino acid sequences of SEQ ID NOs: 140-142 (e.g., TCRa CDRs 1-3), an amino acid sequence having at least 95% identity to the amino acid sequence of SEQ ID NO: 144 (e.g., TCRa full length protein), the amino acid sequences of SEQ ID NOs: 145-147 (e.g., TCRb CDRs 1-3), and an amino acid sequence having at least 95% identity to the amino acid sequence of SEQ ID NO: 149 (e.g., TCRb full length protein). In some embodiments, a TCR comprises the amino acid sequences of SEQ ID NOs: 140-142 (e.g., TCRa CDRs 1-3), an amino acid sequence having at least 95% identity to the amino acid sequence of SEQ ID NO: 286 (e.g., TCRa full length protein), the amino acid sequences of SEQ ID NOs: 145-147 (e.g., TCRb CDRs 1-3), and an amino acid sequence having at least 95% identity to the amino acid sequence of SEQ ID NO: 287 (e.g., TCRb full length protein).

In some embodiments, a TCR comprises the amino acid sequences of SEQ ID NOs: 140-142 (e.g., TCRa CDRs 1-3), an amino acid sequence having at least 98% identity to the amino acid sequence of SEQ ID NO: 143 (e.g., TCRa variable domain), the amino acid sequences of SEQ ID NOs: 145-147 (e.g., TCRb CDRs 1-3), and an amino acid sequence having at least 98% identity to the amino acid sequence of SEQ ID NO: 148 (e.g., TCRb variable domain). In some embodiments, a TCR comprises the amino acid sequences of SEQ ID NOs: 140-142 (e.g., TCRa CDRs 1-3), an amino acid sequence having at least 98% identity to the amino acid sequence of SEQ ID NO: 144 (e.g., TCRa full length protein), the amino acid sequences of SEQ ID NOs: 145-147 (e.g., TCRb CDRs 1-3), and an amino acid sequence having at least 98% identity to the amino acid sequence of SEQ ID NO: 149 (e.g., TCRb full length protein). In some embodiments, a TCR comprises the amino acid sequences of SEQ ID NOs: 140-142 (e.g., TCRa CDRs 1-3), an amino acid sequence having at least 98% identity to the amino acid sequence of SEQ ID NO: 286 (e.g., TCRa full length protein), the amino acid sequences of SEQ ID NOs: 145-147 (e.g., TCRb CDRs 1-3), and an amino acid sequence having at least 98% identity to the amino acid sequence of SEQ ID NO: 287 (e.g., TCRb full length protein).

In some embodiments, a TCR comprises the amino acid sequences of SEQ ID NOs: 150-152 (e.g., TCRa CDRs 1-3), an amino acid sequence having at least 90% identity to the amino acid sequence of SEQ ID NO: 153 (e.g., TCRa variable domain), the amino acid sequences of SEQ ID NOs: 155-157 (e.g., TCRb CDRs 1-3), and an amino acid sequence having at least 90% identity to the amino acid sequence of SEQ ID NO: 158 (e.g., TCRb variable domain). In some embodiments, a TCR comprises the amino acid sequences of SEQ ID NOs: 150-152 (e.g., TCRa CDRs 1-3), an amino acid sequence having at least 90% identity to the amino acid sequence of SEQ ID NO: 154 (e.g., TCRa full length protein), the amino acid sequences of SEQ ID NOs: 155-157 (e.g., TCRb CDRs 1-3), and an amino acid sequence having at least 90% identity to the amino acid sequence of SEQ ID NO: 159 (e.g., TCRb full length protein). In some embodiments, a TCR comprises the amino acid sequences of SEQ ID NOs: 150-152 (e.g., TCRa CDRs 1-3), an amino acid sequence having at least 90% identity to the amino acid sequence of SEQ ID NO: 288 (e.g., TCRa full length protein), the amino acid sequences of SEQ ID NOs: 155-157 (e.g., TCRb CDRs 1-3), and an amino acid sequence having at least 90% identity to the amino acid sequence of SEQ ID NO: 289 (e.g., TCRb full length protein).

In some embodiments, a TCR comprises the amino acid sequences of SEQ ID NOs: 150-152 (e.g., TCRa CDRs 1-3), an amino acid sequence having at least 95% identity to the amino acid sequence of SEQ ID NO: 153 (e.g., TCRa variable domain), the amino acid sequences of SEQ ID NOs: 155-157 (e.g., TCRb CDRs 1-3), and an amino acid sequence having at least 95% identity to the amino acid sequence of SEQ ID NO: 158 (e.g., TCRb variable domain). In some embodiments, a TCR comprises the amino acid sequences of SEQ ID NOs: 150-152 (e.g., TCRa CDRs 1-3), an amino acid sequence having at least 95% identity to the amino acid sequence of SEQ ID NO: 154 (e.g., TCRa full length protein), the amino acid sequences of SEQ ID NOs: 155-157 (e.g., TCRb CDRs 1-3), and an amino acid sequence having at least 95% identity to the amino acid sequence of SEQ ID NO: 159 (e.g., TCRb full length protein). In some embodiments, a TCR comprises the amino acid sequences of SEQ ID NOs: 150-152 (e.g., TCRa CDRs 1-3), an amino acid sequence having at least 95% identity to the amino acid sequence of SEQ ID NO: 288 (e.g., TCRa full length protein), the amino acid sequences of SEQ ID NOs: 155-157 (e.g., TCRb CDRs 1-3), and an amino acid sequence having at least 95% identity to the amino acid sequence of SEQ ID NO: 289 (e.g., TCRb full length protein).

In some embodiments, a TCR comprises the amino acid sequences of SEQ ID NOS: 150-152 (e.g., TCRa CDRs 1-3), an amino acid sequence having at least 98% identity to the amino acid sequence of SEQ ID NO: 153 (e.g., TCRa variable domain), the amino acid sequences of SEQ ID NOs: 155-157 (e.g., TCRb CDRs 1-3), and an amino acid sequence having at least 98% identity to the amino acid sequence of SEQ ID NO: 158 (e.g., TCRb variable domain). In some embodiments, a TCR comprises the amino acid sequences of SEQ ID NOs: 150-152 (e.g., TCRa CDRs 1-3), an amino acid sequence having at least 98% identity to the amino acid sequence of SEQ ID NO: 154 (e.g., TCRa full length protein), the amino acid sequences of SEQ ID NOs: 155-157 (e.g., TCRb CDRs 1-3), and an amino acid sequence having at least 98% identity to the amino acid sequence of SEQ ID NO: 159 (e.g., TCRb full length protein). In some embodiments, a TCR comprises the amino acid sequences of SEQ ID NOs: 150-152 (e.g., TCRa CDRs 1-3), an amino acid sequence having at least 98% identity to the amino acid sequence of SEQ ID NO: 290 (e.g., TCRa full length protein), the amino acid sequences of SEQ ID NOs: 155-157 (e.g., TCRb CDRs 1-3), and an amino acid sequence having at least 98% identity to the amino acid sequence of SEQ ID NO: 291 (e.g., TCRb full length protein).

In some embodiments, a TCR comprises the amino acid sequences of SEQ ID NOs: 160-162 (e.g., TCRa CDRs 1-3), an amino acid sequence having at least 90% identity to the amino acid sequence of SEQ ID NO: 163 (e.g., TCRa variable domain), the amino acid sequences of SEQ ID NOs: 165-167 (e.g., TCRb CDRs 1-3), and an amino acid sequence having at least 90% identity to the amino acid sequence of SEQ ID NO: 168 (e.g., TCRb variable domain). In some embodiments, a TCR comprises the amino acid sequences of SEQ ID NOs: 160-162 (e.g., TCRa CDRs 1-3), an amino acid sequence having at least 90% identity to the amino acid sequence of SEQ ID NO: 164 (e.g., TCRa full length protein), the amino acid sequences of SEQ ID NOs: 165-167 (e.g., TCRb CDRs 1-3), and an amino acid sequence having at least 90% identity to the amino acid sequence of SEQ ID NO: 169 (e.g., TCRb full length protein). In some embodiments, a TCR comprises the amino acid sequences of SEQ ID NOs: 160-162 (e.g., TCRa CDRs 1-3), an amino acid sequence having at least 90% identity to the amino acid sequence of SEQ ID NO: 290 (e.g., TCRa full length protein), the amino acid sequences of SEQ ID NOs: 165-167 (e.g., TCRb CDRs 1-3), and an amino acid sequence having at least 90% identity to the amino acid sequence of SEQ ID NO: 291 (e.g., TCRb full length protein).

In some embodiments, a TCR comprises the amino acid sequences of SEQ ID NOs: 160-162 (e.g., TCRa CDRs 1-3), an amino acid sequence having at least 95% identity to the amino acid sequence of SEQ ID NO: 163 (e.g., TCRa variable domain), the amino acid sequences of SEQ ID NOs: 165-167 (e.g., TCRb CDRs 1-3), and an amino acid sequence having at least 95% identity to the amino acid sequence of SEQ ID NO: 168 (e.g., TCRb variable domain). In some embodiments, a TCR comprises the amino acid sequences of SEQ ID NOs: 160-162 (e.g., TCRa CDRs 1-3), an amino acid sequence having at least 95% identity to the amino acid sequence of SEQ ID NO: 164 (e.g., TCRa full length protein), the amino acid sequences of SEQ ID NOs: 165-167 (e.g., TCRb CDRs 1-3), and an amino acid sequence having at least 95% identity to the amino acid sequence of SEQ ID NO: 169 (e.g., TCRb full length protein). In some embodiments, a TCR comprises the amino acid sequences of SEQ ID NOs: 160-162 (e.g., TCRa CDRs 1-3), an amino acid sequence having at least 95% identity to the amino acid sequence of SEQ ID NO: 290 (e.g., TCRa full length protein), the amino acid sequences of SEQ ID NOs: 165-167 (e.g., TCRb CDRs 1-3), and an amino acid sequence having at least 95% identity to the amino acid sequence of SEQ ID NO: 291 (e.g., TCRb full length protein).

In some embodiments, a TCR comprises the amino acid sequences of SEQ ID NOs: 160-162 (e.g., TCRa CDRs 1-3), an amino acid sequence having at least 98% identity to the amino acid sequence of SEQ ID NO: 163 (e.g., TCRa variable domain), the amino acid sequences of SEQ ID NOs: 165-167 (e.g., TCRb CDRs 1-3), and an amino acid sequence having at least 98% identity to the amino acid sequence of SEQ ID NO: 168 (e.g., TCRb variable domain). In some embodiments, a TCR comprises the amino acid sequences of SEQ ID NOs: 160-162 (e.g., TCRa CDRs 1-3), an amino acid sequence having at least 98% identity to the amino acid sequence of SEQ ID NO: 164 (e.g., TCRa full length protein), the amino acid sequences of SEQ ID NOs: 165-167 (e.g., TCRb CDRs 1-3), and an amino acid sequence having at least 98% identity to the amino acid sequence of SEQ ID NO: 169 (e.g., TCRb full length protein). In some embodiments, a TCR comprises the amino acid sequences of SEQ ID NOs: 160-162 (e.g., TCRa CDRs 1-3), an amino acid sequence having at least 98% identity to the amino acid sequence of SEQ ID NO: 290 (e.g., TCRa full length protein), the amino acid sequences of SEQ ID NOs: 165-167 (e.g., TCRb CDRs 1-3), and an amino acid sequence having at least 98% identity to the amino acid sequence of SEQ ID NO: 291 (e.g., TCRb full length protein).

TABLE 1

| | | | SEQ |
|---|---|---|---|
| | | | ID |
| | Name | Amino Acid Sequence | NO: |
| TCR-A | TCRa | ETRDTTYYL | 1 |
| | CDR1 | | |
| | TCRa | RRNSFDEQNE | 2 |
| | CDR2 | | |
| | TCRa | CALSENRGGTASKLTF | 3 |
| | CDR3 | | |
| | TCRa | AQKVTQAQTEISVVEKEDVTLDCVYETRDTTYYLFWYKQPPS | 4 |
| | variable | GELVFLIRRNSFDEQNEISGRYSWNFQKSTSSFNFTITASQVVD | |
| | domain | SAVYFCALSENRGGTASKLTFGTGTRLQVTL | |
| | TCRa full | AQKVTQAQTEISVVEKEDVTLDCVYETRDTTYYLFWYKQPPS | 5 |
| | | GELVFLIRRNSFDEQNEISGRYSWNFQKSTSSFNFTITASQVVD | |
| | | SAVYFCALSENRGGTASKLTFGTGTRLQVTLNIQNPDPAVYQL | |
| | | RDSKSSDKSVCLFTDFDSQTNVSQSKDSDVYITDKTVLDMRS | |
| | | MDFKSNSAVAWSNKSDFACANAFNNSIIPEDTFFPSPESSCDVK | |
| | | LVEKSFETDTNLNFQNLSVIGFRILLLKVAGFNLLMTLRLWSS | |
| | TCRa full | AQKVTQAQTEISVVEKEDVTLDCVYETRDTTYYLFWYKQPPS | 260 |
| | (DS) | GELVFLIRRNSFDEQNEISGRYSWNFQKSTSSFNFTITASQVVD | |
| | | SAVYFCALSENRGGTASKLTFGTGTRLQVTLNIQNPDPAVYQL | |
| | | RDSKSSDKSVCLFTDFDSQTNVSQSKDSDVYITDKCVLDMRS | |
| | | MDFKSNSAVAWSNKSDFACANAFNNSIIPEDTFFPSPESSCDVK | |
| | | LVEKSFETDTNLNFQNLSVIGFRILLLKVAGFNLLMTLRLWSS | |
| | TCRb | ISGHRSV | 6 |
| | CDR1 | | |
| | TCRb | EYFSETQR | 7 |
| | CDR2 | | |
| | TCRb | CASSLVGGPSSEAFF | 8 |
| | CDR3 | | |
| | TCRb | KAGVTQTPRYLIKTRGQQVTLSCSPISGHRSVSWYQQTPGQGL | 9 |
| | variable | QFLFEYFSETQRNKGNFPGRFSGRQFSNSRSEMNVSTLELGDS | |
| | domain | ALYLCASSLVGGPSSEAFFGQGTRLTVV | |
| | TCRb full | KAGVTQTPRYLIKTRGQQVTLSCSPISGHRSVSWYQQTPGQGL | 10 |
| | | QFLFEYFSETQRNKGNFPGRFSGRQFSNSRSEMNVSTLELGDS | |
| | | ALYLCASSLVGGPSSEAFFGQGTRLTVVEDLKNVFPPEVAVFE | |
| | | PSEAEISHTQKATLVCLATGFYPDHVELSWWVNGKEVHSGVS | |
| | | TDPQPLKEQPALNDSRYCLSSRLRVSATFWQNPRNHFRCQVQF | |
| | | YGLSENDEWTQDRAKPVTQIVSAEAWGRADCGFTSESYQQG | |
| | | VLSATILYEILLGKATLYAVLVSALVLMAMVKRKDSRG | |
| | TCRb full | KAGVTQTPRYLIKTRGQQVTLSCSPISGHRSVSWYQQTPGQGL | 261 |
| | (DS) | QFLFEYFSETQRNKGNFPGRFSGRQFSNSRSEMNVSTLELGDS | |
| | | ALYLCASSLVGGPSSEAFFGQGTRLTVVEDLKNVFPPEVAVFE | |
| | | PSEAEISHTQKATLVCLATGFYPDHVELSWWVNGKEVHSGVC | |
| | | TDPQPLKEQPALNDSRYCLSSRLRVSATFWQNPRNHFRCQVQF | |
| | | YGLSENDEWTQDRAKPVTQIVSAEAWGRADCGFTSESYQQG | |
| | | VLSATILYEILLGKATLYAVLVSALVLMAMVKRKDSRG | |
| TCR-B | TCRa | ETRDTTYYL | 11 |
| | CDR1 | | |
| | TCRa | RRNSFDEQNE | 12 |
| | CDR2 | | |
| | TCRa | CALSEEGGGANSKLTF | 13 |
| | CDR3 | | |
| | TCRa | AQKVTQAQTEISVVEKEDVTLDCVYETRDTTYYLFWYKQPPS | 14 |
| | variable | GELVFLIRRNSFDEQNEISGRYSWNFQKSTSSFNFTITASQVVD | |
| | domain | SAVYFCALSEEGGGANSKLTFGKGITLSVRP | |
| | TCRa full | AQKVTQAQTEISVVEKEDVTLDCVYETRDTTYYLFWYKQPPS | 15 |
| | | GELVFLIRRNSFDEQNEISGRYSWNFQKSTSSFNFTITASQVVD | |
| | | SAVYFCALSEEGGGANSKLTFGKGITLSVRPNIQNPDPAVYQL | |
| | | RDSKSSDKSVCLFTDFDSQTNVSQSKDSDVYITDKTVLDMRS | |
| | | MDFKSNSAVAWSNKSDFACANAFNNSIIPEDTFFPSPESSCDVK | |
| | | LVEKSFETDTNLNFQNLSVIGFRILLLKVAGFNLLMTLRLWSS | |
| | TCRa full | AQKVTQAQTEISVVEKEDVTLDCVYETRDTTYYLFWYKQPPS | 262 |
| | (DS) | GELVFLIRRNSFDEQNEISGRYSWNFQKSTSSFNFTITASQVVD | |
| | | SAVYFCALSEEGGGANSKLTFGKGITLSVRPNIQNPDPAVYQL | |
| | | RDSKSSDKSVCLFTDFDSQTNVSQSKDSDVYITDKCVLDMRS | |
| | | MDFKSNSAVAWSNKSDFACANAFNNSIIPEDTFFPSPESSCDVK | |
| | | LVEKSFETDTNLNFQNLSVIGFRILLLKVAGFNLLMTLRLWSS | |
| | TCRb | ISGHRSV | 16 |
| | CDR1 | | |
| | TCRb | EYFSETQR | 17 |
| | CDR2 | | |
| | TCRb | CASSLAGGANSPLHF | 18 |
| | CDR3 | | |
| | TCRb | KAGVTQTPRYLIKTRGQQVTLSCSPISGHRSVSWYQQTPGQGL | 19 |
| | variable | QFLFEYFSETQRNKGNFPGRFSGRQFSNSRSEMNVSTLELGDS | |
| | domain | ALYLCASSLAGGANSPLHFGNGTRLTVT | |
| | TCRb full | KAGVTQTPRYLIKTRGQQVTLSCSPISGHRSVSWYQQTPGQGL | 20 |
| | | QFLFEYFSETQRNKGNFPGRFSGRQFSNSRSEMNVSTLELGDS | |
| | | ALYLCASSLAGGANSPLHFGNGTRLTVTEDLKNVFPPEVAVFE | |
| | | PSEAEISHTQKATLVCLATGFYPDHVELSWWVNGKEVHSGVS | |
| | | TDPQPLKEQPALNDSRYCLSSRLRVSATFWQNPRNHFRCQVQF | |
| | | YGLSENDEWTQDRAKPVTQIVSAEAWGRADCGFTSESYQQG | |
| | | VLSATILYEILLGKATLYAVLVSALVLMAMVKRKDSRG | |
| | TCRb full | KAGVTQTPRYLIKTRGQQVTLSCSPISGHRSVSWYQQTPGQGL | 263 |
| | (DS) | QFLFEYFSETQRNKGNFPGRFSGRQFSNSRSEMNVSTLELGDS | |
| | | ALYLCASSLAGGANSPLHFGNGTRLTVTEDLKNVFPPEVAVFE | |
| | | PSEAEISHTQKATLVCLATGFYPDHVELSWWVNGKEVHSGVC | |
| | | TDPQPLKEQPALNDSRYCLSSRLRVSATFWQNPRNHFRCQVQF | |
| | | YGLSENDEWTQDRAKPVTQIVSAEAWGRADCGFTSESYQQG | |
| | | VLSATILYEILLGKATLYAVLVSALVLMAMVKRKDSRG | |
| TCR-C | TCRa | SSIFNTW | 21 |
| | CDR1 | | |
| | TCRa | ALYKAGELT | 22 |
| | CDR2 | | |
| | TCRa | CAGHSIIQGAQKLVF | 23 |
| | CDR3 | | |
| | TCRa | GQQLNQSPQSMFIQEGEDVSMNCTSSSIFNTWLWYKQEPGEGP | 24 |
| | variable | VLLIALYKAGELTSNGRLTAQFGITRKDSFLNISASIPSDVGIYF | |
| | domain | CAGHSIIQGAQKLVFGQGTRLTINP | |
| | TCRa full | GQQLNQSPQSMFIQEGEDVSMNCTSSSIFNTWLWYKQDPGEG | 25 |
| | | PVLLIALYKAGELTSNGRLTAQFGITRKDSFLNISASIPSDVGIY | |
| | | FCAGHSIIQGAQKLVFGQGTRLTINPNIQNPDPAVYQLRDSKSS | |
| | | DKSVCLFTDFDSQTNVSQSKDSDVYITDKTVLDMRSMDFKSN | |
| | | SAVAWSNKSDFACANAFNNSIIPEDTFFPSPESSCDVKLVEKSF | |
| | | ETDTNLNFQNLSVIGFRILLLKVAGFNLLMTLRLWSS | |
| | TCRa full | GQQLNQSPQSMFIQEGEDVSMNCTSSSIFNTWLWYKQDPGEG | 264 |
| | (DS) | PVLLIALYKAGELTSNGRLTAQFGITRKDSFLNISASIPSDVGIY | |
| | | FCAGHSIIQGAQKLVFGQGTRLTINPNIQNPDPAVYQLRDSKSS | |
| | | DKSVCLFTDFDSQTNVSQSKDSDVYITDKCVLDMRSMDFKSN | |
| | | SAVAWSNKSDFACANAFNNSIIPEDTFFPSPESSCDVKLVEKSF | |
| | | ETDTNLNFQNLSVIGFRILLLKVAGFNLLMTLRLWSS | |
| | TCRb | ISGHRSV | 26 |
| | CDR1 | | |
| | TCRb | EYFSETQR | 27 |
| | CDR2 | | |
| | TCRb | CASGRSSYNEQFF | 28 |
| | CDR3 | | |
| | TCRb | KAGVTQTPRYLIKTRGQQVTLSCSPISGHRSVSWYQQTPGQGL | 29 |
| | variable | QFLFEYFSETQRNKGNFPGRFSGRQFSNSRSEMNVSTLELGDS | |
| | domain | ALYLCASGRSSYNEQFFGPGTRLTVL | |
| | TCRb full | KAGVTQTPRYLIKTRGQQVTLSCSPISGHRSVSWYQQTPGQGL | 30 |
| | | QFLFEYFSETQRNKGNFPGRFSGRQFSNSRSEMNVSTLELGDS | |
| | | ALYLCASGRSSYNEQFFGPGTRLTVLEDLKNVFPPEVAVFEPSE | |
| | | AEISHTQKATLVCLATGFYPDHVELSWWVNGKEVHSGVSTDP | |
| | | QPLKEQPALNDSRYCLSSRLRVSATFWQNPRNHFRCQVQFYG | |
| | | LSENDEWTQDRAKPVTQIVSAEAWGRADCGFTSESYQQGVLS | |
| | | ATILYEILLGKATLYAVLVSALVLMAMVKRKDSRG | |
| | TCRb full | KAGVTQTPRYLIKTRGQQVTLSCSPISGHRSVSWYQQTPGQGL | 265 |
| | (DS) | QFLFEYFSETQRNKGNFPGRFSGRQFSNSRSEMNVSTLELGDS | |
| | | ALYLCASGRSSYNEQFFGPGTRLTVLEDLKNVFPPEVAVFEPSE | |
| | | AEISHTQKATLVCLATGFYPDHVELSWWVNGKEVHSGVCTDP | |
| | | QPLKEQPALNDSRYCLSSRLRVSATFWQNPRNHFRCQVQFYG | |
| | | LSENDEWTQDRAKPVTQIVSAEAWGRADCGFTSESYQQGVLS | |
| | | ATILYEILLGKATLYAVLVSALVLMAMVKRKDSRG | |
| TCR-D | TCRa | TATGYPSL | 31 |
| | CDR1 | | |
| | TCRa | KATKADDKG | 32 |
| | CDR2 | | |
| | TCRa | CALSDHAGGTSYGKLTF | 33 |
| | CDR3 | | |
| | TCRa | GDSVTQMEGPVTLSEEAFLTINCTYTATGYPSLFWYVQYPGEG | 34 |
| | variable | LQLLLKATKADDKGSNKGFEATYRKETTSFHLEKGSVQVSDSA | |
| | domain | VYFCALSDHAGGTSYGKLTFGQGTILTVHP | |
| | TCRa full | MNYSPGLVSLILLLLGRTRGDSVTQMEGPVTLSEEAFLTINCTY | 35 |
| | | TATGYPSLFWYVQYPGEGLQLLLKATKADDKGSNKGFEATYR | |
| | | KETTSFHLEKGSVQVSDSAVYFCALSDHAGGTSYGKLTFGQGT | |
| | | ILTVHPNIQNPDPAVYQLRDSKSSDKSVCLFTDFDSQTNVSQSK | |
| | | DSDVYITDKTVLDMRSMDFKSNSAVAWSNKSDFACANAFNNS | |
| | | IIPEDTFFPSPESSCDVKLVEKSFETDTNLNFQNLSVIGFRILLLK | |
| | | VAGFNLLMTLRLWSS | |
| | TCRa full | MNYSPGLVSLILLLLGRTRGDSVTQMEGPVTLSEEAFLTINCTY | 266 |
| | (DS) | TATGYPSLFWYVQYPGEGLQLLLKATKADDKGSNKGFEATYR | |
| | | KETTSFHLEKGSVQVSDSAVYFCALSDHAGGTSYGKLTFGQGT | |
| | | ILTVHPNIQNPDPAVYQLRDSKSSDKSVCLFTDFDSQTNVSQSK | |
| | | DSDVYITDKCVLDMRSMDFKSNSAVAWSNKSDFACANAFNNS | |
| | | IIPEDTFFPSPESSCDVKLVEKSFETDTNLNFQNLSVIGFRILLLK | |
| | | VAGFNLLMTLRLWSS | |
| | TCRb | DMNHEYM | 36 |
| | CDR1 | | |
| | TCRb | YSVGAGIT | 37 |
| | CDR2 | | |
| | TCRb | CASMSPATNEKLFF | 38 |
| | CDR3 | | |
| | TCRb | NAGVTQTPKFQVLKTGQSMTLQCAQDMNHEYMSWYRQDPG | 39 |
| | variable | MGLRLIHYSVGAGITDQGEVPNGYNVSRSTTEDFPLRLLSAAPS | |
| | domain | QTSVYFCASMSPATNEKLFFGSGTQLSVL | |
| | TCRb full | MSIGLLCCAALSLLWAGPVNAGVTQTPKFQVLKTGQSMTLQC | 40 |
| | | AQDMNHEYMSWYRQDPGMGLRLIHYSVGAGITDQGEVPNGY | |
| | | NVSRSTTEDFPLRLLSAAPSQTSVYFCASMSPATNEKLFFGSGT | |
| | | QLSVLEDLNKVFPPEVAVFEPSEAEISHTQKATLVCLATGFFPD | |
| | | HVELSWWVNGKEVHSGVSTDPQPLKEQPALNDSRYCLSSRLR | |
| | | VSATFWQNPRNHFRCQVQFYGLSENDEWTQDRAKPVTQIVSA | |
| | | EAWGRADCGFTSVSYQQGVLSATILYEILLGKATLYAVLVSAL | |
| | | VLMAMVKRKDF | |
| | TCRb full | MSIGLLCCAALSLLWAGPVNAGVTQTPKFQVLKTGQSMTLQC | 267 |
| | (DS) | AQDMNHEYMSWYRQDPGMGLRLIHYSVGAGITDQGEVPNGY | |
| | | NVSRSTTEDFPLRLLSAAPSQTSVYFCASMSPATNEKLFFGSGT | |
| | | QLSVLEDLNKVFPPEVAVFEPSEAEISHTQKATLVCLATGFFPD | |
| | | HVELSWWVNGKEVHSGVCTDPQPLKEQPALNDSRYCLSSRLR | |
| | | VSATFWQNPRNHFRCQVQFYGLSENDEWTQDRAKPVTQIVSA | |
| | | EAWGRADCGFTSVSYQQGVLSATILYEILLGKATLYAVLVSAL | |
| | | VLMAMVKRKDF | |
| TCR-E | TCRa | SSSVPPYL | 41 |
| | CDR1 | | |
| | TCRa | KYTSAATLVK | 42 |
| | CDR2 | | |
| | TCRa | CAVSDQNSGYSTLTF | 43 |
| | CDR3 | | |
| | TCRa | AQSVTQLGSHVSVSEGALVLLRCNYSSSVPPYLFWYVQYPNQ | 44 |
| | variable | GLQLLLKYTSAATLVKGINGFEAEFKKSETSFHLTKPSAHMSD | |
| | domain | AAEYFCAVSDQNSGYSTLTFGKGTMLLVSP | |
| | TCRa full | MLLLLVPVLEVIFTLGGTRAQSVTQLGSHVSVSEGALVLLRCN | 45 |
| | | YSSSVPPYLFWYVQYPNQGLQLLLKYTSAATLVKGINGFEAEF | |
| | | KKSETSFHLTKPSAHMSDAAEYFCAVSDQNSGYSTLTFGKGTM | |
| | | LLVSPDIQNPDPAVYQLRDSKSSDKSVCLFTDFDSQTNVSQSKD | |
| | | SDVYITDKTVLDMRSMDFKSNSAVAWSNKSDFACANAFNNSII | |
| | | PEDTFFPSPESSCDVKLVEKSFETDTNLNFQNLSVIGFRILLLKV | |
| | | AGFNLLMTLRLWSS | |
| | TCRa full | MLLLLVPVLEVIFTLGGTRAQSVTQLGSHVSVSEGALVLLRCN | 268 |
| | (DS) | YSSSVPPYLFWYVQYPNQGLQLLLKYTSAATLVKGINGFEAEF | |
| | | KKSETSFHLTKPSAHMSDAAEYFCAVSDQNSGYSTLTFGKGTM | |
| | | LLVSPDIQNPDPAVYQLRDSKSSDKSVCLFTDFDSQTNVSQSKD | |
| | | SDVYITDKCVLDMRSMDFKSNSAVAWSNKSDFACANAFNNSII | |
| | | PEDTFFPSPESSCDVKLVEKSFETDTNLNFQNLSVIGFRILLLKV | |
| | | AGFNLLMTLRLWSS | |
| | TCRb | ISGHRSV | 46 |
| | CDR1 | | |
| | TCRb | EYFSETQR | 47 |
| | CDR2 | | |
| | TCRb | CASSFKGATNEKLFF | 48 |
| | CDR3 | | |
| | TCRb | KAGVTQTPRYLIKTRGQQVTLSCSPISGHRSVSWYQQTPGQGL | 49 |
| | variable | QFLFEYFSETQRNKGNFPGRFSGRQFSNSRSEMNVSTLELGDSA | |
| | domain | LYLCASSFKGATNEKLFFGSGTQLSVL | |
| | TCRb full | MGSRLLCWVLLCLLGAGPVKAGVTQTPRYLIKTRGQQVTLSC | 50 |
| | | SPISGHRSVSWYQQTPGQGLQFLFEYFSETQRNKGNFPGRFSGR | |
| | | QFSNSRSEMNVSTLELGDSALYLCASSFKGATNEKLFFGSGTQL | |
| | | SVLEDLNKVFPPEVAVFEPSEAEISHTQKATLVCLATGFFPDHV | |
| | | ELSWWVNGKEVHSGVSTDPQPLKEQPALNDSRYCLSSRLRVS | |
| | | ATFWQNPRNHFRCQVQFYGLSENDEWTQDRAKPVTQIVSAEA | |
| | | WGRADCGFTSVSYQQGVLSATILYEILLGKATLYAVLVSALVL | |
| | | MAMVKRKDF | |
| | TCRb full | MGSRLLCWVLLCLLGAGPVKAGVTQTPRYLIKTRGQQVTLSC | 269 |
| | (DS) | SPISGHRSVSWYQQTPGQGLQFLFEYFSETQRNKGNFPGRFSGR | |
| | | QFSNSRSEMNVSTLELGDSALYLCASSFKGATNEKLFFGSGTQL | |
| | | SVLEDLNKVFPPEVAVFEPSEAEISHTQKATLVCLATGFFPDHV | |
| | | ELSWWVNGKEVHSGVCTDPQPLKEQPALNDSRYCLSSRLRVS | |
| | | ATFWQNPRNHFRCQVQFYGLSENDEWTQDRAKPVTQIVSAEA | |
| | | WGRADCGFTSVSYQQGVLSATILYEILLGKATLYAVLVSALVL | |
| | | MAMVKRKDF | |
| TCR-F | TCRa | TNYSPAYL | 51 |
| | CDR1 | | |
| | TCRa | LIRENEKEK | 52 |
| | CDR2 | | |
| | TCRa | CALGGVGSGGYNKLIF | 53 |
| | CDR3 | | |
| | TCRa | SQKIEQNSEALNIQEGKTATLTCNYTNYSPAYLQWYRQDPGRG | 54 |
| | variable | PVFLLLIRENEKEKRKERLKVTFDTTLKQSLFHITASQPADSAT | |
| | domain | YLCALGGVGSGGYNKLIFGAGTRLAVHP | |
| | TCRa full | MESFLGGVLLILWLQVDWVKSQKIEQNSEALNIQEGKTATLTC | 55 |
| | | NYTNYSPAYLQWYRQDPGRGPVFLLLIRENEKEKRKERLKVTF | |
| | | DTTLKQSLFHITASQPADSATYLCALGGVGSGGYNKLIFGAGT | |
| | | RLAVHPYIQNPDPAVYQLRDSKSSDKSVCLFTDFDSQTNVSQS | |
| | | KDSDVYITDKTVLDMRSMDFKSNSAVAWSNKSDFACANAFN | |
| | | NSIIPEDTFFPSPESSCDVKLVEKSFETDTNLNFQNLSVIGFRILLL | |
| | | KVAGFNLLMTLRLWSS | |
| | TCRa full | MESFLGGVLLILWLQVDWVKSQKIEQNSEALNIQEGKTATLTC | 270 |
| | (DS) | NYTNYSPAYLQWYRQDPGRGPVFLLLIRENEKEKRKERLKVTF | |
| | | DTTLKQSLFHITASQPADSATYLCALGGVGSGGYNKLIFGAGT | |
| | | RLAVHPYIQNPDPAVYQLRDSKSSDKSVCLFTDFDSQTNVSQS | |
| | | KDSDVYITDKCVLDMRSMDFKSNSAVAWSNKSDFACANAFN | |
| | | NSIIPEDTFFPSPESSCDVKLVEKSFETDTNLNFQNLSVIGFRILLL | |
| | | KVAGFNLLMTLRLWSS | |
| | TCRb | HMGHRAM | 56 |
| | CDR1 | | |
| | TCRb | VYSYEKLS | 57 |
| | CDR2 | | |
| | TCRb | CASSQDNHAGELFF | 58 |
| | CDR3 | | |
| | TCRb | DTEVTQTPKHLVMGMTNKKSLKCEQHMGHRAMYWYKQKAK | 59 |
| | variable | KPPELMFVYSYEKLSINESVPSRFSPECPNSSLLNLHLHALQPED | |
| | domain | SALYLCASSQDNHAGELFFGEGSRLTVL | |
| | TCRb full | MGCRLLCCAVLCLLGAVPIDTEVTQTPKHLVMGMTNKKSLKC | 60 |
| | | EQHMGHRAMYWYKQKAKKPPELMFVYSYEKLSINESVPSRFS | |
| | | PECPNSSLLNLHLHALQPEDSALYLCASSQDNHAGELFFGEGSR | |
| | | LTVLEDLKNVFPPEVAVFEPSEAEISHTQKATLVCLATGFYPDH | |
| | | VELSWWVNGKEVHSGVSTDPQPLKEQPALNDSRYCLSSRLRV | |
| | | SATFWQNPRNHFRCQVQFYGLSENDEWTQDRAKPVTQIVSAE | |
| | | AWGRADCGFTSESYQQGVLSATILYEILLGKATLYAVLVSALV | |
| | | LMAMVKRKDSRG | |
| | TCRb full | MGCRLLCCAVLCLLGAVPIDTEVTQTPKHLVMGMTNKKSLKC | 271 |
| | (DS) | EQHMGHRAMYWYKQKAKKPPELMFVYSYEKLSINESVPSRFS | |
| | | PECPNSSLLNLHLHALQPEDSALYLCASSQDNHAGELFFGEGSR | |
| | | LTVLEDLKNVFPPEVAVFEPSEAEISHTQKATLVCLATGFYPDH | |
| | | VELSWWVNGKEVHSGVCTDPQPLKEQPALNDSRYCLSSRLRV | |
| | | SATFWQNPRNHFRCQVQFYGLSENDEWTQDRAKPVTQIVSAE | |
| | | AWGRADCGFTSESYQQGVLSATILYEILLGKATLYAVLVSALV | |
| | | LMAMVKRKDSRG | |
| TCR-G | TCRa | TATGYPSL | 61 |
| | CDR1 | | |
| | TCRa | KATKADDKG | 62 |
| | CDR2 | | |
| | TCRa | CALSDPGGGSYIPTF | 63 |
| | CDR3 | | |
| | TCRa | GDSVTQMEGPVTLSEEAFLTINCTYTATGYPSLFWYVQYPGEG | 64 |
| | variable | LQLLLKATKADDKGSNKGFEATYRKETTSFHLEKGSVQVSDS | |
| | domain | AVYFCALSDPGGGSYIPTFGRGTSLIVHP | |
| | TCRa full | MNYSPGLVSLILLLLGRTRGDSVTQMEGPVTLSEEAFLTINCTY | 65 |
| | | TATGYPSLFWYVQYPGEGLQLLLKATKADDKGSNKGFEATYR | |
| | | KETTSFHLEKGSVQVSDSAVYFCALSDPGGGSYIPTFGRGTSLI | |
| | | VHPYIQNPDPAVYQLRDSKSSDKSVCLFTDFDSQTNVSQSKDS | |
| | | DVYITDKTVLDMRSMDFKSNSAVAWSNKSDFACANAFNNSIIP | |
| | | EDTFFPSPESSCDVKLVEKSFETDTNLNFQNLSVIGFRILLLKVA | |
| | | GFNLLMTLRLWSS | |
| | TCRa full | MNYSPGLVSLILLLLGRTRGDSVTQMEGPVTLSEEAFLTINCTY | 272 |
| | (DS) | TATGYPSLFWYVQYPGEGLQLLLKATKADDKGSNKGFEATYR | |
| | | KETTSFHLEKGSVQVSDSAVYFCALSDPGGGSYIPTFGRGTSLI | |
| | | VHPYIQNPDPAVYQLRDSKSSDKSVCLFTDFDSQTNVSQSKDS | |
| | | DVYITDKCVLDMRSMDFKSNSAVAWSNKSDFACANAFNNSIIP | |
| | | EDTFFPSPESSCDVKLVEKSFETDTNLNFQNLSVIGFRILLLKVA | |
| | | GFNLLMTLRLWSS | |
| | TCRb | TLNHNVM | 66 |
| | CDR1 | | |
| | TCRb | HYYDKDFN | 67 |
| | CDR2 | | |
| | TCRb | CATSRAGDDLALQFF | 68 |
| | CDR3 | | |
| | TCRb | DAMVIQNPRYQVTQFGKPVTLSCSQTLNHNVMYWYQQKSSQ | 69 |
| | variable | APKLLFHYYDKDFNNEADTPDNFQSRRPNTSFCFLDIRSPGLGD | |
| | domain | AAMYLCATSRAGDDLALQFFGPGTRLTVL | |
| | TCRb full | MGPGLLHWMALCLLGTGHGDAMVIQNPRYQVTQFGKPVTLS | 70 |
| | | CSQTLNHNVMYWYQQKSSQAPKLLFHYYDKDFNNEADTPDN | |
| | | FQSRRPNTSFCFLDIRSPGLGDAAMYLCATSRAGDDLALQFFGP | |
| | | GTRLTVLEDLKNVFPPEVAVFEPSEAEISHTQKATLVCLATGFY | |
| | | PDHVELSWWVNGKEVHSGVSTDPQPLKEQPALNDSRYCLSSR | |
| | | LRVSATFWQNPRNHFRCQVQFYGLSENDEWTQDRAKPVTQIV | |
| | | SAEAWGRADCGFTSESYQQGVLSATILYEILLGKATLYAVLVS | |
| | | ALVLMAMVKRKDSRG | |
| | TCRb full | MGPGLLHWMALCLLGTGHGDAMVIQNPRYQVTQFGKPVTLS | 273 |
| | (DS) | CSQTLNHNVMYWYQQKSSQAPKLLFHYYDKDFNNEADTPDN | |
| | | FQSRRPNTSFCFLDIRSPGLGDAAMYLCATSRAGDDLALQFFGP | |
| | | GTRLTVLEDLKNVFPPEVAVFEPSEAEISHTQKATLVCLATGFY | |
| | | PDHVELSWWVNGKEVHSGVCTDPQPLKEQPALNDSRYCLSSR | |
| | | LRVSATFWQNPRNHFRCQVQFYGLSENDEWTQDRAKPVTQIV | |
| | | SAEAWGRADCGFTSESYQQGVLSATILYEILLGKATLYAVLVS | |
| | | ALVLMAMVKRKDSRG | |
| TCR-H | TCRa | QTSGFYGL | 71 |
| | CDR1 | | |
| | TCRa | YNALDGLE | 72 |
| | CDR2 | | |
| | TCRa | CAVKKGSSNTGKLIF | 73 |
| | CDR3 | | |
| | TCRa | GQSLEQPSEVTAVEGAIVQINCTYQTSGFYGLSWYQQHDGGAP | 74 |
| | variable | TFLSYNALDGLEETGRFSSFLSRSDSYGYLLLQELQMKDSASYF | |
| | domain | CAVKKGSSNTGKLIFGQGTTLQVKP | |
| | TCRa full | MWGAFLLYVSMKMGGTAGQSLEQPSEVTAVEGAIVQINCTYQ | 75 |
| | | TSGFYGLSWYQQHDGGAPTFLSYNALDGLEETGRFSSFLSRSD | |
| | | SYGYLLLQELQMKDSASYFCAVKKGSSNTGKLIFGQGTTLQV | |
| | | KPDIQNPDPAVYQLRDSKSSDKSVCLFTDFDSQTNVSQSKDSD | |
| | | VYITDKTVLDMRSMDFKSNSAVAWSNKSDFACANAFNNSIIPE | |
| | | DTFFPSPESSCDVKLVEKSFETDTNLNFQNLSVIGFRILLLKVAG | |
| | | FNLLMTLRLWSS | |
| | TCRa full | MWGAFLLYVSMKMGGTAGQSLEQPSEVTAVEGAIVQINCTYQ | 274 |
| | (DS) | TSGFYGLSWYQQHDGGAPTFLSYNALDGLEETGRFSSFLSRSD | |
| | | SYGYLLLQELQMKDSASYFCAVKKGSSNTGKLIFGQGTTLQV | |
| | | KPDIQNPDPAVYQLRDSKSSDKSVCLFTDFDSQTNVSQSKDSD | |
| | | VYITDKCVLDMRSMDFKSNSAVAWSNKSDFACANAFNNSIIPE | |
| | | DTFFPSPESSCDVKLVEKSFETDTNLNFQNLSVIGFRILLLKVAG | |
| | | FNLLMTLRLWSS | |
| | TCRb | ISGHDNL | 76 |
| | CDR1 | | |
| | TCRb | HFVKESKQ | 77 |
| | CDR2 | | |
| | TCRb | CASSPLQGYEQYF | 78 |
| | CDR3 | | |
| | TCRb | EAGVTQFPSHSVIEKGQTVTLRCDPISGHDNLYWYRRVMGKEI | 79 |
| | variable | KFLLHFVKESKQDESGMPNNRFLAERTGGTYSTLKVQPAELED | |
| | domain | SGVYFCASSPLQGYEQYFGPGTRLTVT | |
| | TCRb full | MVSRLLSLVSLCLLGAKHIEAGVTQFPSHSVIEKGQTVTLRCDP | 80 |
| | | ISGHDNLYWYRRVMGKEIKFLLHFVKESKQDESGMPNNRFLA | |
| | | ERTGGTYSTLKVQPAELEDSGVYFCASSPLQGYEQYFGPGTRL | |
| | | TVTEDLKNVFPPEVAVFEPSEAEISHTQKATLVCLATGFYPDHV | |
| | | ELSWWVNGKEVHSGVSTDPQPLKEQPALNDSRYCLSSRLRVS | |
| | | ATFWQNPRNHFRCQVQFYGLSENDEWTQDRAKPVTQIVSAEA | |
| | | WGRADCGFTSESYQQGVLSATILYEILLGKATLYAVLVSALVL | |
| | | MAMVKRKDSRG | |
| | TCRb full | MVSRLLSLVSLCLLGAKHIEAGVTQFPSHSVIEKGQTVTLRCDP | 275 |
| | (DS) | ISGHDNLYWYRRVMGKEIKFLLHFVKESKQDESGMPNNRFLA | |
| | | ERTGGTYSTLKVQPAELEDSGVYFCASSPLQGYEQYFGPGTRL | |
| | | TVTEDLKNVFPPEVAVFEPSEAEISHTQKATLVCLATGFYPDHV | |
| | | ELSWWVNGKEVHSGVCTDPQPLKEQPALNDSRYCLSSRLRVS | |
| | | ATFWQNPRNHFRCQVQFYGLSENDEWTQDRAKPVTQIVSAEA | |
| | | WGRADCGFTSESYQQGVLSATILYEILLGKATLYAVLVSALVL | |
| | | MAMVKRKDSRG | |
| TCR-I | TCRa | SNSASQSF | 81 |
| | CDR1 | | |
| | TCRa | SVYSSGN | 82 |
| | CDR2 | | |
| | TCRa | CVVNRRNARLMF | 83 |
| | CDR3 | | |
| | TCRa | RKEVEQDPGPFNVPEGATVAFNCTYSNSASQSFFWYRQDCRK | 84 |
| | variable | EPKLLMSVYSSGNEDGRFTAQLNRASQYISLLIRDSKLSDSATY | |
| | domain | LCVVNRRNARLMFGDGTQLVVKP | |
| | TCRa full | MISLRVLLVILWLQLSWVWSQRKEVEQDPGPFNVPEGATVAF | 85 |
| | | NCTYSNSASQSFFWYRQDCRKEPKLLMSVYSSGNEDGRFTAQ | |
| | | LNRASQYISLLIRDSKLSDSATYLCVVNRRNARLMFGDGTQLV | |
| | | VKPNIQNPDPAVYQLRDSKSSDKSVCLFTDFDSQTNVSQSKDS | |
| | | DVYITDKTVLDMRSMDFKSNSAVAWSNKSDFACANAFNNSIIP | |
| | | EDTFFPSPESSCDVKLVEKSFETDTNLNFQNLSVIGFRILLLKVA | |
| | | GFNLLMTLRLWSS | |
| | TCRa full | MISLRVLLVILWLQLSWVWSQRKEVEQDPGPFNVPEGATVAF | 276 |
| | (DS) | NCTYSNSASQSFFWYRQDCRKEPKLLMSVYSSGNEDGRFTAQ | |
| | | LNRASQYISLLIRDSKLSDSATYLCVVNRRNARLMFGDGTQLV | |
| | | VKPNIQNPDPAVYQLRDSKSSDKSVCLFTDFDSQTNVSQSKDS | |
| | | DVYITDKCVLDMRSMDFKSNSAVAWSNKSDFACANAFNNSIIP | |
| | | EDTFFPSPESSCDVKLVEKSFETDTNLNFQNLSVIGFRILLLKVA | |
| | | GFNLLMTLRLWSS | |
| | TCRb | TKGHDRM | 86 |
| | CDR1 | | |
| | TCRb | YSFDVKDI | 87 |
| | CDR2 | | |
| | TCRb | CATSDGDGQETQYF | 88 |
| | CDR3 | | |
| | TCRb | DADVTQTPRNRITKTGKRIMLECSQTKGHDRMYWYRQDPGLG | 89 |
| | variable | LQLIYYSFDVKDINKGEISDGYSVSRQAQAKFSLSLESAIPNQTA | |
| | domain | LYFCATSDGDGQETQYFGPGTRLLVL | |
| | TCRb full | MASLLFFCGAFYLLGTGSMDADVTQTPRNRITKTGKRIMLECS | 90 |
| | | QTKGHDRMYWYRQDPGLGLQLIYYSFDVKDINKGEISDGYSV | |
| | | SRQAQAKFSLSLESAIPNQTALYFCATSDGDGQETQYFGPGTRL | |
| | | LVLEDLKNVFPPEVAVFEPSEAEISHTQKATLVCLATGFYPDHV | |
| | | ELSWWVNGKEVHSGVSTDPQPLKEQPALNDSRYCLSSRLRVS | |
| | | ATFWQNPRNHFRCQVQFYGLSENDEWTQDRAKPVTQIVSAEA | |
| | | WGRADCGFTSESYQQGVLSATILYEILLGKATLYAVLVSALVL | |
| | | MAMVKRKDSRG | |
| | TCRb full | MASLLFFCGAFYLLGTGSMDADVTQTPRNRITKTGKRIMLECS | 277 |
| | (DS) | QTKGHDRMYWYRQDPGLGLQLIYYSFDVKDINKGEISDGYSV | |
| | | SRQAQAKFSLSLESAIPNQTALYFCATSDGDGQETQYFGPGTRL | |
| | | LVLEDLKNVFPPEVAVFEPSEAEISHTQKATLVCLATGFYPDHV | |
| | | ELSWWVNGKEVHSGVCTDPQPLKEQPALNDSRYCLSSRLRVS | |
| | | ATFWQNPRNHFRCQVQFYGLSENDEWTQDRAKPVTQIVSAEA | |
| | | WGRADCGFTSESYQQGVLSATILYEILLGKATLYAVLVSALVL | |
| | | MAMVKRKDSRG | |
| TCR-J | TCRa | TNYSPAYL | 91 |
| | CDR1 | | |
| | TCRa | LIRENEKEK | 92 |
| | CDR2 | | |
| | TCRa | CAPISGSARQLTF | 93 |
| | CDR3 | | |
| | TCRa | SQKIEQNSEALNIQEGKTATLTCNYTNYSPAYLQWYRQDPGRG | 94 |
| | variable | PVFLLLIRENEKEKRKERLKVTFDTTLKQSLFHITASQPADSAT | |
| | domain | YLCAPISGSARQLTFGSGTQLTVLP | |
| | TCRa full | MESFLGGVLLILWLQVDWVKSQKIEQNSEALNIQEGKTATLTC | 95 |
| | | NYTNYSPAYLQWYRQDPGRGPVFLLLIRENEKEKRKERLKVTF | |
| | | DTTLKQSLFHITASQPADSATYLCAPISGSARQLTFGSGTQLTV | |
| | | LPDIQNPDPAVYQLRDSKSSDKSVCLFTDFDSQTNVSQSKDSD | |
| | | VYITDKTVLDMRSMDFKSNSAVAWSNKSDFACANAFNNSIIPE | |
| | | DTFFPSPESSCDVKLVEKSFETDTNLNFQNLSVIGFRILLLKVAG | |
| | | FNLLMTLRLWSS | |
| | TCRa full | MESFLGGVLLILWLQVDWVKSQKIEQNSEALNIQEGKTATLTC | 278 |
| | (DS) | NYTNYSPAYLQWYRQDPGRGPVFLLLIRENEKEKRKERLKVTF | |
| | | DTTLKQSLFHITASQPADSATYLCAPISGSARQLTFGSGTQLTV | |
| | | LPDIQNPDPAVYQLRDSKSSDKSVCLFTDFDSQTNVSQSKDSD | |
| | | VYITDKCVLDMRSMDFKSNSAVAWSNKSDFACANAFNNSIIPE | |
| | | DTFFPSPESSCDVKLVEKSFETDTNLNFQNLSVIGFRILLLKVAG | |
| | | FNLLMTLRLWSS | |
| | TCRb | NLNHDAM | 96 |
| | CDR1 | | |
| | TCRb | YSQIVNDF | 97 |
| | CDR2 | | |
| | TCRb | CASSMGQGQPQHF | 98 |
| | CDR3 | | |
| | TCRb | DGGITQSPKYLFRKEGQNVTLSCEQNLNHDAMYWYRQDPGQ | 99 |
| | variable | GLRLIYYSQIVNDFQKGDIAEGYSVSREKKESFPLTVTSAQKNP | |
| | domain | TAFYLCASSMGQGQPQHFGDGTRLSIL | |
| | TCRb full | MSNQVLCCVVLCFLGANTVDGGITQSPKYLFRKEGQNVTLSCE | 100 |
| | | QNLNHDAMYWYRQDPGQGLRLIYYSQIVNDFQKGDIAEGYSV | |
| | | SREKKESFPLTVTSAQKNPTAFYLCASSMGQGQPQHFGDGTRL | |
| | | SILEDLNKVFPPEVAVFEPSEAEISHTQKATLVCLATGFFPDHVE | |
| | | LSWWVNGKEVHSGVSTDPQPLKEQPALNDSRYCLSSRLRVSA | |
| | | TFWQNPRNHFRCQVQFYGLSENDEWTQDRAKPVTQIVSAEAW | |
| | | GRADCGFTSVSYQQGVLSATILYEILLGKATLYAVLVSALVLM | |
| | | AMVKRKDF | |
| | TCRb full | MSNQVLCCVVLCFLGANTVDGGITQSPKYLFRKEGQNVTLSCE | 279 |
| | (DS) | QNLNHDAMYWYRQDPGQGLRLIYYSQIVNDFQKGDIAEGYSV | |
| | | SREKKESFPLTVTSAQKNPTAFYLCASSMGQGQPQHFGDGTRL | |
| | | SILEDLNKVFPPEVAVFEPSEAEISHTQKATLVCLATGFFPDHVE | |
| | | LSWWVNGKEVHSGVCTDPQPLKEQPALNDSRYCLSSRLRVSA | |
| | | TFWQNPRNHFRCQVQFYGLSENDEWTQDRAKPVTQIVSAEAW | |
| | | GRADCGFTSVSYQQGVLSATILYEILLGKATLYAVLVSALVLM | |
| | | AMVKRKDF | |
| TCR-K | TCRa | STISGTDYI | 110 |
| | CDR1 | | |
| | TCRa | HGLTSNV | 111 |
| | CDR2 | | |
| | TCRa | CILRDVENYGQNFVF | 112 |
| | CDR3 | | |
| | TCRa | DAKTTQPNSMESNEEEPVHLPCNHSTISGTDYIHWYRQLPSQG | 113 |
| | variable | PEYVIHGLTSNVNNRMASLAIAEDRKSSTLILHRATLRDAAVY | |
| | domain | YCILRDVENYGQNFVFGPGTRLSVLP | |
| | TCRa full | MKLVTSITVLLSLGIMGDAKTTQPNSMESNEEEPVHLPCNHSTI | 114 |
| | | SGTDYIHWYRQLPSQGPEYVIHGLTSNVNNRMASLAIAEDRKS | |
| | | STLILHRATLRDAAVYYCILRDVENYGQNFVFGPGTRLSVLPYI | |
| | | QNPDPAVYQLRDSKSSDKSVCLFTDFDSQTNVSQSKDSDVYIT | |
| | | DKTVLDMRSMDFKSNSAVAWSNKSDFACANAFNNSIIPEDTFF | |
| | | PSPESSCDVKLVEKSFETDTNLNFQNLSVIGFRILLLKVAGFNLL | |
| | | MTLRLWSS | |
| | TCRa full | MKLVTSITVLLSLGIMGDAKTTQPNSMESNEEEPVHLPCNHSTI | 280 |
| | (DS) | SGTDYIHWYRQLPSQGPEYVIHGLTSNVNNRMASLAIAEDRKS | |
| | | STLILHRATLRDAAVYYCILRDVENYGQNFVFGPGTRLSVLPYI | |
| | | QNPDPAVYQLRDSKSSDKSVCLFTDFDSQTNVSQSKDSDVYIT | |
| | | DKCVLDMRSMDFKSNSAVAWSNKSDFACANAFNNSIIPEDTFF | |
| | | PSPESSCDVKLVEKSFETDTNLNFQNLSVIGFRILLLKVAGFNLL | |
| | | MTLRLWSS | |
| | TCRb | ISEHNRL | 115 |
| | CDR1 | | |
| | TCRb | YFQNEAQL | 116 |
| | CDR2 | | |
| | TCRb | CASSLWTSGNSPLHF | 117 |
| | CDR3 | | |
| | TCRb | DTGVSQDPRHKITKRGQNVTFRCDPISEHNRLYWYRQTLGQGP | 118 |
| | variable | EFLTYFQNEAQLEKSRLLSDRFSAERPKGSFSTLEIQRTEQGDS | |
| | domain | AMYLCASSLWTSGNSPLHFGNGTRLTVT | |
| | TCRb full | MGTSLLCWMALCLLGADHADTGVSQDPRHKITKRGQNVTFR | 119 |
| | | CDPISEHNRLYWYRQTLGQGPEFLTYFQNEAQLEKSRLLSDRF | |
| | | SAERPKGSFSTLEIQRTEQGDSAMYLCASSLWTSGNSPLHFGN | |
| | | GTRLTVTEDLNKVFPPEVAVFEPSEAEISHTQKATLVCLATGFF | |
| | | PDHVELSWWVNGKEVHSGVSTDPQPLKEQPALNDSRYCLSSR | |
| | | LRVSATFWQNPRNHFRCQVQFYGLSENDEWTQDRAKPVTQIV | |
| | | SAEAWGRADCGFTSVSYQQGVLSATILYEILLGKATLYAVLVS | |
| | | ALVLMAMVKRKDF | |
| | TCRb full | MGTSLLCWMALCLLGADHADTGVSQDPRHKITKRGQNVTFR | 281 |
| | (DS) | CDPISEHNRLYWYRQTLGQGPEFLTYFQNEAQLEKSRLLSDRF | |
| | | SAERPKGSFSTLEIQRTEQGDSAMYLCASSLWTSGNSPLHFGN | |
| | | GTRLTVTEDLNKVFPPEVAVFEPSEAEISHTQKATLVCLATGFF | |
| | | PDHVELSWWVNGKEVHSGVCTDPQPLKEQPALNDSRYCLSSR | |
| | | LRVSATFWQNPRNHFRCQVQFYGLSENDEWTQDRAKPVTQIV | |
| | | SAEAWGRADCGFTSVSYQQGVLSATILYEILLGKATLYAVLVS | |
| | | ALVLMAMVKRKDF | |
| TCR-L | TCRa | STTLSNI | 120 |
| | CDR1 | | |
| | TCRa | QLVKSGEVK | 121 |
| | CDR2 | | |
| | TCRa | CAPPQTGANNLFF | 122 |
| | CDR3 | | |
| | TCRa | GQQVMQIPQYQHVQEGEDFTTYCNSSTTLSNIQWYKQRPGGH | 123 |
| | variable | PVFLIQLVKSGEVKKQKRLTFQFGEAKKNSSLHITATQTTDVGT | |
| | domain | YFCAPPQTGANNLFFGTGTRLTVIP | |
| | TCRa full | MLLITSMLVLWMQLSQVNGQQVMQIPQYQHVQEGEDFTTYC | 124 |
| | | NSSTTLSNIQWYKQRPGGHPVFLIQLVKSGEVKKQKRLTFQFG | |
| | | EAKKNSSLHITATQTTDVGTYFCAPPQTGANNLFFGTGTRLTVI | |
| | | PYIQNPDPAVYQLRDSKSSDKSVCLFTDFDSQTNVSQSKDSDV | |
| | | YITDKTVLDMRSMDFKSNSAVAWSNKSDFACANAFNNSIIPED | |
| | | TFFPSPESSCDVKLVEKSFETDTNLNFQNLSVIGFRILLLKVAGF | |
| | | NLLMTLRLWSS | |
| | TCRa full | MLLITSMLVLWMQLSQVNGQQVMQIPQYQHVQEGEDFTTYC | 282 |
| | (DS) | NSSTTLSNIQWYKQRPGGHPVFLIQLVKSGEVKKQKRLTFQFG | |
| | | EAKKNSSLHITATQTTDVGTYFCAPPQTGANNLFFGTGTRLTVI | |
| | | PYIQNPDPAVYQLRDSKSSDKSVCLFTDFDSQTNVSQSKDSDV | |
| | | YITDKCVLDMRSMDFKSNSAVAWSNKSDFACANAFNNSIIPED | |
| | | TFFPSPESSCDVKLVEKSFETDTNLNFQNLSVIGFRILLLKVAGF | |
| | | NLLMTLRLWSS | |
| | TCRb | ISGHRSV | 125 |
| | CDR1 | | |
| | TCRb | EYFSETQR | 126 |
| | CDR2 | | |
| | TCRb | CASSLVGGGDTEAFF | 127 |
| | CDR3 | | |
| | TCRb | KAGVTQTPRYLIKTRGQQVTLSCSPISGHRSVSWYQQTPGQGL | 128 |
| | variable | QFLFEYFSETQRNKGNFPGRFSGRQFSNSRSEMNVSTLELGDSA | |
| | domain | LYLCASSLVGGGDTEAFFGQGTRLTVV | |
| | TCRb full | MGSRLLCWVLLCLLGAGPVKAGVTQTPRYLIKTRGQQVTLSC | 129 |
| | | SPISGHRSVSWYQQTPGQGLQFLFEYFSETQRNKGNFPGRFSGR | |
| | | QFSNSRSEMNVSTLELGDSALYLCASSLVGGGDTEAFFGQGTR | |
| | | LTVVEDLNKVFPPEVAVFEPSEAEISHTQKATLVCLATGFFPDH | |
| | | VELSWWVNGKEVHSGVSTDPQPLKEQPALNDSRYCLSSRLRV | |
| | | SATFWQNPRNHFRCQVQFYGLSENDEWTQDRAKPVTQIVSAE | |
| | | AWGRADCGFTSVSYQQGVLSATILYEILLGKATLYAVLVSALV | |
| | | LMAMVKRKDF | |
| | TCRb full | MGSRLLCWVLLCLLGAGPVKAGVTQTPRYLIKTRGQQVTLSC | 283 |
| | (DS) | SPISGHRSVSWYQQTPGQGLQFLFEYFSETQRNKGNFPGRFSGR | |
| | | QFSNSRSEMNVSTLELGDSALYLCASSLVGGGDTEAFFGQGTR | |
| | | LTVVEDLNKVFPPEVAVFEPSEAEISHTQKATLVCLATGFFPDH | |
| | | VELSWWVNGKEVHSGVCTDPQPLKEQPALNDSRYCLSSRLRV | |
| | | SATFWQNPRNHFRCQVQFYGLSENDEWTQDRAKPVTQIVSAE | |
| | | AWGRADCGFTSVSYQQGVLSATILYEILLGKATLYAVLVSALV | |
| | | LMAMVKRKDF | |
| TCR-M | TCRa | TNYSPAYL | 130 |
| | CDR1 | | |
| | TCRa | LIRENEKEK | 131 |
| | CDR2 | | |
| | TCRa | CALPTTDSWGKLQF | 132 |
| | CDR3 | | |
| | TCRa | SQKIEQNSEALNIQEGKTATLTCNYTNYSPAYLQWYRQDPGRG | 133 |
| | variable | PVFLLLIRENEKEKRKERLKVTFDTTLKQSLFHITASQPADSAT | |
| | domain | YLCALPTTDSWGKLQFGAGTQVVVTP | |
| | TCRa full | MESFLGGVLLILWLQVDWVKSQKIEQNSEALNIQEGKTATLTC | 134 |
| | | NYTNYSPAYLQWYRQDPGRGPVFLLLIRENEKEKRKERLKVTF | |
| | | DTTLKQSLFHITASQPADSATYLCALPTTDSWGKLQFGAGTQV | |
| | | VVTPDIQNPDPAVYQLRDSKSSDKSVCLFTDFDSQTNVSQSKD | |
| | | SDVYITDKTVLDMRSMDFKSNSAVAWSNKSDFACANAFNNSII | |
| | | PEDTFFPSPESSCDVKLVEKSFETDTNLNFQNLSVIGFRILLLKV | |
| | | AGFNLLMTLRLWSS | |
| | TCRa full | MESFLGGVLLILWLQVDWVKSQKIEQNSEALNIQEGKTATLTC | 284 |
| | (DS) | NYTNYSPAYLQWYRQDPGRGPVFLLLIRENEKEKRKERLKVTF | |
| | | DTTLKQSLFHITASQPADSATYLCALPTTDSWGKLQFGAGTQV | |
| | | VVTPDIQNPDPAVYQLRDSKSSDKSVCLFTDFDSQTNVSQSKD | |
| | | SDVYITDKCVLDMRSMDFKSNSAVAWSNKSDFACANAFNNSII | |
| | | PEDTFFPSPESSCDVKLVEKSFETDTNLNFQNLSVIGFRILLLKV | |
| | | AGFNLLMTLRLWSS | |
| | TCRb | ISGHRSV | 135 |
| | CDR1 | | |
| | TCRb | EYFSETQR | 136 |
| | CDR2 | | |
| | TCRb | CASSFKDREETQYF | 137 |
| | CDR3 | | |
| | TCRb | KAGVTQTPRYLIKTRGQQVTLSCSPISGHRSVSWYQQTPGQGL | 138 |
| | variable | QFLFEYFSETQRNKGNFPGRFSGRQFSNSRSEMNVSTLELGDSA | |
| | domain | LYLCASSFKDREETQYFGPGTRLLVL | |
| | TCRb full | MGSRLLCWVLLCLLGAGPVKAGVTQTPRYLIKTRGQQVTLSC | 139 |
| | | SPISGHRSVSWYQQTPGQGLQFLFEYFSETQRNKGNFPGRFSGR | |
| | | QFSNSRSEMNVSTLELGDSALYLCASSFKDREETQYFGPGTRLL | |
| | | VLEDLKNVFPPEVAVFEPSEAEISHTQKATLVCLATGFYPDHVE | |
| | | LSWWVNGKEVHSGVSTDPQPLKEQPALNDSRYCLSSRLRVSA | |
| | | TFWQNPRNHFRCQVQFYGLSENDEWTQDRAKPVTQIVSAEAW | |
| | | GRADCGFTSESYQQGVLSATILYEILLGKATLYAVLVSALVLM | |
| | | AMVKRKDSRG | |
| | TCRb full | MGSRLLCWVLLCLLGAGPVKAGVTQTPRYLIKTRGQQVTLSC | 285 |
| | (DS) | SPISGHRSVSWYQQTPGQGLQFLFEYFSETQRNKGNFPGRFSGR | |
| | | QFSNSRSEMNVSTLELGDSALYLCASSFKDREETQYFGPGTRLL | |
| | | VLEDLKNVFPPEVAVFEPSEAEISHTQKATLVCLATGFYPDHVE | |
| | | LSWWVNGKEVHSGVCTDPQPLKEQPALNDSRYCLSSRLRVSA | |
| | | TFWQNPRNHFRCQVQFYGLSENDEWTQDRAKPVTQIVSAEAW | |
| | | GRADCGFTSESYQQGVLSATILYEILLGKATLYAVLVSALVLM | |
| | | AMVKRKDSRG | |
| TCR-N | TCRa | SNSASDYF | 140 |
| | CDR1 | | |
| | TCRa | DIRSNMDKR | 141 |
| | CDR2 | | |
| | TCRa | CAENGGNNARLMF | 142 |
| | CDR3 | | |
| | TCRa | GESVGLHLPTLSVQEGDNSIINCAYSNSASDYFIWYKQESGKGP | 143 |
| | variable | QFIIDIRSNMDKRQGQRVTVLLNKTVKHLSLQIAATQPGDSAV | |
| | domain | YFCAENGGNNARLMFGDGTQLVVKP | |
| | TCRa full | MAGIRALFMYLWLQLDWVSRGESVGLHLPTLSVQEGDNSIINC | 144 |
| | | AYSNSASDYFIWYKQESGKGPQFIIDIRSNMDKRQGQRVTVLL | |
| | | NKTVKHLSLQIAATQPGDSAVYFCAENGGNNARLMFGDGTQL | |
| | | VVKPNIQNPDPAVYQLRDSKSSDKSVCLFTDFDSQTNVSQSKD | |
| | | SDVYITDKTVLDMRSMDFKSNSAVAWSNKSDFACANAFNNSII | |
| | | PEDTFFPSPESSCDVKLVEKSFETDTNLNFQNLSVIGFRILLLKV | |
| | | AGFNLLMTLRLWSS | |
| | TCRa full | MAGIRALFMYLWLQLDWVSRGESVGLHLPTLSVQEGDNSIINC | 286 |
| | (DS) | AYSNSASDYFIWYKQESGKGPQFIIDIRSNMDKRQGQRVTVLL | |
| | | NKTVKHLSLQIAATQPGDSAVYFCAENGGNNARLMFGDGTQL | |
| | | VVKPNIQNPDPAVYQLRDSKSSDKSVCLFTDFDSQTNVSQSKD | |
| | | SDVYITDKCVLDMRSMDFKSNSAVAWSNKSDFACANAFNNSII | |
| | | PEDTFFPSPESSCDVKLVEKSFETDTNLNFQNLSVIGFRILLLKV | |
| | | AGFNLLMTLRLWSS | |
| | TCRb | ISGHRSV | 145 |
| | CDR1 | | |
| | TCRb | EYFSETQR | 146 |
| | CDR2 | | |
| | TCRb | CASSATGGDQPQHF | 147 |
| | CDR3 | | |
| | TCRb | KAGVTQTPRYLIKTRGQQVTLSCSPISGHRSVSWYQQTPGQGL | 148 |
| | variable | QFLFEYFSETQRNKGNFPGRFSGRQFSNSRSEMNVSTLELGDSA | |
| | domain | LYLCASSATGGDQPQHFGDGTRLSIL | |
| | TCRb full | MGSRLLCWVLLCLLGAGPVKAGVTQTPRYLIKTRGQQVTLSC | 149 |
| | | SPISGHRSVSWYQQTPGQGLQFLFEYFSETQRNKGNFPGRFSGR | |
| | | QFSNSRSEMNVSTLELGDSALYLCASSATGGDQPQHFGDGTRL | |
| | | SILEDLNKVFPPEVAVFEPSEAEISHTQKATLVCLATGFFPDHVE | |
| | | LSWWVNGKEVHSGVSTDPQPLKEQPALNDSRYCLSSRLRVSA | |
| | | TFWQNPRNHFRCQVQFYGLSENDEWTQDRAKPVTQIVSAEAW | |
| | | GRADCGFTSVSYQQGVLSATILYEILLGKATLYAVLVSALVLM | |
| | | AMVKRKDF | |
| | TCRb full | MGSRLLCWVLLCLLGAGPVKAGVTQTPRYLIKTRGQQVTLSC | 287 |
| | (DS) | SPISGHRSVSWYQQTPGQGLQFLFEYFSETQRNKGNFPGRFSGR | |
| | | QFSNSRSEMNVSTLELGDSALYLCASSATGGDQPQHFGDGTRL | |
| | | SILEDLNKVFPPEVAVFEPSEAEISHTQKATLVCLATGFFPDHVE | |
| | | LSWWVNGKEVHSGVCTDPQPLKEQPALNDSRYCLSSRLRVSA | |
| | | TFWQNPRNHFRCQVQFYGLSENDEWTQDRAKPVTQIVSAEAW | |
| | | GRADCGFTSVSYQQGVLSATILYEILLGKATLYAVLVSALVLM | |
| | | AMVKRKDF | |
| TCR-O | TCRa | STTSDRL | 150 |
| | CDR1 | | |
| | TCRa | VLLSNGAVK | 151 |
| | CDR2 | | |
| | TCRa | CAVDMTGSSGDKLTF | 152 |
| | CDR3 | | |
| | TCRa | ELKVEQNPLFLSMQEGKNYTIYCNYSTTSDRLYWYRQDPGKS | 153 |
| | variable | LESLFVLLSNGAVKQEGRLMASLDTKARLSTLHITAAVHDLSA | |
| | domain | TYFCAVDMTGSSGDKLTFGTGTRLAVRP | |
| | TCRa full | MKKLLAMILWLQLDRLSGELKVEQNPLFLSMQEGKNYTIYCN | 154 |
| | | YSTTSDRLYWYRQDPGKSLESLFVLLSNGAVKQEGRLMASLD | |
| | | TKARLSTLHITAAVHDLSATYFCAVDMTGSSGDKLTFGTGTRL | |
| | | AVRPNIQNPDPAVYQLRDSKSSDKSVCLFTDFDSQTNVSQSKD | |
| | | SDVYITDKTVLDMRSMDFKSNSAVAWSNKSDFACANAFNNSII | |
| | | PEDTFFPSPESSCDVKLVEKSFETDTNLNFQNLSVIGFRILLLKV | |
| | | AGFNLLMTLRLWSS | |
| | TCRa full | MKKLLAMILWLQLDRLSGELKVEQNPLFLSMQEGKNYTIYCN | 288 |
| | (DS) | YSTTSDRLYWYRQDPGKSLESLFVLLSNGAVKQEGRLMASLD | |
| | | TKARLSTLHITAAVHDLSATYFCAVDMTGSSGDKLTFGTGTRL | |
| | | AVRPNIQNPDPAVYQLRDSKSSDKSVCLFTDFDSQTNVSQSKD | |
| | | SDVYITDKCVLDMRSMDFKSNSAVAWSNKSDFACANAFNNSII | |
| | | PEDTFFPSPESSCDVKLVEKSFETDTNLNFQNLSVIGFRILLLKV | |
| | | AGFNLLMTLRLWSS | |
| | TCRb | ISGHVSL | 155 |
| | CDR1 | | |
| | TCRb | YFQNEAQL | 156 |
| | CDR2 | | |
| | TCRb | CASSLVAGHGGDGYTF | 157 |
| | CDR3 | | |
| | TCRb | GAGVSQSPRYKVAKRGQDVALRCDPISGHVSLFWYQQALGQG | 158 |
| | variable | PEFLTYFQNEAQLDKSGLPSDRFFAERPEGSVSTLKIQRTQKED | |
| | domain | SAVYLCASSLVAGHGGDGYTFGSGTRLTVV | |
| | TCRb full | MGTRLLCWVVLGFLGTDHTGAGVSQSPRYKVAKRGQDVALR | 159 |
| | | CDPISGHVSLFWYQQALGQGPEFLTYFQNEAQLDKSGLPSDRF | |
| | | FAERPEGSVSTLKIQRTQKEDSAVYLCASSLVAGHGGDGYTFG | |
| | | SGTRLTVVEDLNKVFPPEVAVFEPSEAEISHTQKATLVCLATGF | |
| | | FPDHVELSWWVNGKEVHSGVSTDPQPLKEQPALNDSRYCLSS | |
| | | RLRVSATFWQNPRNHFRCQVQFYGLSENDEWTQDRAKPVTQI | |
| | | VSAEAWGRADCGFTSVSYQQGVLSATILYEILLGKATLYAVLV | |
| | | SALVLMAMVKRKDF | |
| | TCRb full | MGTRLLCWVVLGFLGTDHTGAGVSQSPRYKVAKRGQDVALR | 289 |
| | (DS) | CDPISGHVSLFWYQQALGQGPEFLTYFQNEAQLDKSGLPSDRF | |
| | | FAERPEGSVSTLKIQRTQKEDSAVYLCASSLVAGHGGDGYTFG | |
| | | SGTRLTVVEDLNKVFPPEVAVFEPSEAEISHTQKATLVCLATGF | |
| | | FPDHVELSWWVNGKEVHSGVCTDPQPLKEQPALNDSRYCLSS | |
| | | RLRVSATFWQNPRNHFRCQVQFYGLSENDEWTQDRAKPVTQI | |
| | | VSAEAWGRADCGFTSVSYQQGVLSATILYEILLGKATLYAVLV | |
| | | SALVLMAMVKRKDF | |
| TCR-P | TCRa | STTLSNI | 160 |
| | CDR1 | | |
| | TCRa | QLVKSGEVK | 161 |
| | CDR2 | | |
| | TCRa | CAGLGGSNYKLTF | 162 |
| | CDR3 | | |
| | TCRa | GQQVMQIPQYQHVQEGEDFTTYCNSSTTLSNIQWYKQRPGGH | 163 |
| | variable | PVFLIQLVKSGEVKKQKRLTFQFGEAKKNSSLHITATQTTDVGT | |
| | domain | YFCAGLGGSNYKLTFGKGTLLTVNP | |
| | TCRa full | MLLITSMLVLWMQLSQVNGQQVMQIPQYQHVQEGEDFTTYC | 164 |
| | | NSSTTLSNIQWYKQRPGGHPVFLIQLVKSGEVKKQKRLTFQFG | |
| | | EAKKNSSLHITATQTTDVGTYFCAGLGGSNYKLTFGKGTLLTV | |
| | | NPNIQNPDPAVYQLRDSKSSDKSVCLFTDFDSQTNVSQSKDSD | |
| | | VYITDKTVLDMRSMDFKSNSAVAWSNKSDFACANAFNNSIIPE | |
| | | DTFFPSPESSCDVKLVEKSFETDTNLNFQNLSVIGFRILLLKVAG | |
| | | FNLLMTLRLWSS | |
| | TCRa full | MLLITSMLVLWMQLSQVNGQQVMQIPQYQHVQEGEDFTTYC | 290 |
| | (DS) | NSSTTLSNIQWYKQRPGGHPVFLIQLVKSGEVKKQKRLTFQFG | |
| | | EAKKNSSLHITATQTTDVGTYFCAGLGGSNYKLTFGKGTLLTV | |
| | | NPNIQNPDPAVYQLRDSKSSDKSVCLFTDFDSQTNVSQSKDSD | |
| | | VYITDKCVLDMRSMDFKSNSAVAWSNKSDFACANAFNNSIIPE | |
| | | DTFFPSPESSCDVKLVEKSFETDTNLNFQNLSVIGFRILLLKVAG | |
| | | FNLLMTLRLWSS | |
| | TCRb | NLNHDAM | 165 |
| | CDR1 | | |
| | TCRb | YSQIVNDF | 166 |
| | CDR2 | | |
| | TCRb | CASSPFSTDTQYF | 167 |
| | CDR3 | | |
| | TCRb | DGGITQSPKYLFRKEGQNVTLSCEQNLNHDAMYWYRQDPGQ | 168 |
| | variable | GLRLIYYSQIVNDFQKGDIAEGYSVSREKKESFPLTVTSAQKNP | |
| | domain | TAFYLCASSPFSTDTQYFGPGTRLTVL | |
| | TCRb full | MSNQVLCCVVLCFLGANTVDGGITQSPKYLFRKEGQNVTLSCE | 169 |
| | | QNLNHDAMYWYRQDPGQGLRLIYYSQIVNDFQKGDIAEGYSV | |
| | | SREKKESFPLTVTSAQKNPTAFYLCASSPFSTDTQYFGPGTRLT | |
| | | VLEDLKNVFPPEVAVFEPSEAEISHTQKATLVCLATGFYPDHVE | |
| | | LSWWVNGKEVHSGVSTDPQPLKEQPALNDSRYCLSSRLRVSA | |
| | | TFWQNPRNHFRCQVQFYGLSENDEWTQDRAKPVTQIVSAEAW | |
| | | GRADCGFTSESYQQGVLSATILYEILLGKATLYAVLVSALVLM | |
| | | AMVKRKDSRG | |
| | TCRb full | MSNQVLCCVVLCFLGANTVDGGITQSPKYLFRKEGQNVTLSCE | 291 |
| | (DS) | QNLNHDAMYWYRQDPGQGLRLIYYSQIVNDFQKGDIAEGYSV | |
| | | SREKKESFPLTVTSAQKNPTAFYLCASSPFSTDTQYFGPGTRLT | |
| | | VLEDLKNVFPPEVAVFEPSEAEISHTQKATLVCLATGFYPDHVE | |
| | | LSWWVNGKEVHSGVCTDPQPLKEQPALNDSRYCLSSRLRVSA | |
| | | TFWQNPRNHFRCQVQFYGLSENDEWTQDRAKPVTQIVSAEAW | |
| | | GRADCGFTSESYQQGVLSATILYEILLGKATLYAVLVSALVLM | |
| | | AMVKRKDSRG | |
| | TRAC | NIQNPDPAVYQLRDSKSSDKSVCLFTDFDSQTNVSQSKDSDVYI | 106 |
| | (alpha | TDKTVLDMRSMDFKSNSAVAWSNKSDFACANAFNNSIIPEDTF | |
| | constant | FPSPESSCDVKLVEKSFETDTNLNFQNLSVIGFRILLLKVAGFNL | |
| | domain) | LMTLRLWSS | |
| | TRBC1 | EDLNKVFPPEVAVFEPSEAEISHTQKATLVCLATGFFPDHVELS | 107 |
| | (beta | WWVNGKEVHSGVSTDPQPLKEQPALNDSRYCLSSRLRVSATF | |
| | constant | WQNPRNHFRCQVQFYGLSENDEWTQDRAKPVTQIVSAEAWG | |
| | domain) | RADCGFTSVSYQQGVLSATILYEILLGKATLYAVLVSALVLMA | |
| | | MVKRKDF | |
| | TRBC2 | EDLKNVFPPEVAVFEPSEAEISHTQKATLVCLATGFYPDHVELS | 108 |
| | (beta | WWVNGKEVHSGVSTDPQPLKEQPALNDSRYCLSSRLRVSATF | |
| | constant | WQNPRNHFRCQVQFYGLSENDEWTQDRAKPVTQIVSAEAWG | |
| | domain) | RADCGFTSESYQQGVLSATILYEILLGKATLYAVLVSALVLMA | |
| | | MVKRKDSRG | |
| | GAD65 | NFFRMVISNPAAT | 109 |
| | 555-567 | | |

In some embodiments, a TCR is an exogenous TCR. An exogenous TCR may be any TCR that is introduced to a T cell, e.g., a regulatory T cell, wherein the TCR is not endogenous to (i.e., naturally occurring in) that regulatory T cell. For example, in some embodiments, an exogenous TCR is encoded by a nucleic acid that is not endogenous to a regulatory T cell (i.e., not naturally occurring in the genome of the regulatory T cell). In some embodiments, the nucleic acid is an engineered nucleic acid, for example, a recombinant or synthetic nucleic acid. Thus, an exogenous TCR may also be referred to herein as an engineered TCR. A regulatory T cell comprising (e.g., expressing) an engineered TCR is considered an engineered regulatory T cell.

The TCR may be a human TCR. In some embodiments, the TCR is a TCR from a monkey, mouse, rat, or any other animal. In some embodiments, a TCR is chimeric (comprises nucleic acid and/or amino acid sequences from two or more different species or from two or more different TCRs of the same species).

A target peptide may be a peptide, or a portion of a peptide, that is associated with Type 1 Diabetes. A target peptide associated with Type 1 Diabetes may be a peptide belonging to a GAD65. In some embodiments, a target peptide is a peptide that is overexpressed in a population of cells associated with Type 1 Diabetes relative to a control (e.g., relative to a population of cells that are not associated with Type 1 Diabetes). In some embodiments, a target peptide is a peptide that is expressed at the site of disease relative to other sites in the body.

In some embodiments, a target peptide is a GAD65 peptide. As described herein, a GAD65 peptide is a peptide of GAD65. In some embodiments, a GAD65 peptide comprises the sequence of NFFRMVISNPAAT (SEQ ID NO: 109). In some embodiments, a GAD65 peptide consists of the sequence of SEQ ID NO: 109. Thus, in some embodiments, a TCR of the disclosure binds specifically to a peptide comprising or consisting of the sequence of SEQ ID NO: 109.

Glutamate decarboxylase 65 (GAD65) is a 585 amino acid, 65 kDa protein, that is an isoform of the GAD enzyme, which is a major enzyme in the synthesis of the neurotransmitter γ-amino butyric acid (GABA). GAD and GABA are mainly present in nerve cells but, are also detected in non-neural cells and organs such as the islet cells of the pancreas, adrenal gland, testis, and ovaries. GAD has two major protein isoforms that catalyze GABA synthesis. One isoform has a molecular size of 65 kDa (GAD65), while the other has a molecular size of 67 kDa size (GAD67). GAD65 is the product of a gene located on Chromosome 10 and GAD67 on Chromosome 2. In the pancreas, GAD65 is mostly detected in synaptic-like vesicles. GAD65 is initially synthesized in the cytosol rather than the ER and exhibits different translational regulation than proinsulin and other insulin secretory granule autoantigens.

In some embodiments, a target peptide is subject to an increased autoimmune reaction in a subject having Type 1 Diabetes relative to a control. In some embodiments, a target peptide is a peptide that is present at a site associated with Type 1 Diabetes within a subject relative to unaffected sites within the same subject. In some embodiments, a target peptide is specifically presented by an MHC allele that is associated with the presence of Type 1 Diabetes in the subject.

In some embodiments, a target peptide is a peptide that is overexpressed in cells of a subject having Type 1 Diabetes relative to a control (e.g., relative to a healthy subject). A target peptide is considered to be overexpressed in cells (e.g., associated with Type 1 Diabetes) if expression of the target peptide in the cells is at least 10%, at least 15%, at least 20%, at least 25%, at least 30%, at least 40%, at least 50%, at least 60%, at least 70%, at least 80%, or at least 90% higher in the cells relative to control cells (e.g., a population of cells that are not associated with Type 1 Diabetes). In some embodiments, a target peptide is overexpressed in cells of a subject having Type 1 Diabetes if expression of the target peptide in cells of the subject having Type 1 Diabetes is at least 10%, at least 15%, at least 20%, at least 25%, at least 30%, at least 40%, at least 50%, at least 60%, at least 70%, at least 80%, or at least 90% higher in the subject having Type 1 Diabetes relative to a healthy subject (e.g., a subject that does not have Type 1 Diabetes).

In some embodiments, a target peptide is a peptide that is highly expressed in cells at the site of disease in a subject having Type 1 Diabetes relative to a control (e.g., target peptide expression in an unaffected/non-disease site in the subject). A target peptide is considered to be highly expressed in cells (e.g., associated with Type 1 Diabetes) at the site of disease if expression of the target peptide in the cells the site of disease is at least 10%, at least 15%, at least 20%, at least 25%, at least 30%, at least 40%, at least 50%, at least 60%, at least 70%, at least 80%, or at least 90% higher in the cells relative to control cells (e.g., cells not at the site of disease).

In some embodiments, a target peptide is a peptide that is complexed with an MHC having an HLA haplotype associated with Type 1 Diabetes.

A TCR, in some embodiments, is (or is encoded as) a single polypeptide (e.g., comprising a beta chain and an alpha chain). In some embodiments, a TCR comprises an N-terminal beta chain and a C-terminal alpha chain. In other embodiments, a TCR comprises an N-terminal alpha chain and a C-terminal beta chain.

A TCR may comprise a linker domain positioned between an alpha chain and a beta chain. In some embodiments, a linker domain comprises a self-cleaving peptide sequence (e.g., a self-cleaving peptide sequence positioned between an alpha chain and a beta chain). A self-cleaving peptide sequence is a peptide sequence that induces a polypeptide to separate into two peptides using a non-classical mechanism. In some embodiments, a self-cleaving peptide sequence can induce ribosomal skipping during translation of a polypeptide. In some embodiments, a self-cleaving peptide sequence is 10-30, 10-25, 15-30, 15-25, or 18-22 amino acids in length. In some embodiments, a self-cleaving peptide sequence may be a 2A peptide sequence. A 2A peptide sequence may comprise, for example, a DXEXNPGP (SEQ ID NO: 101) amino acid motif, wherein X can be any amino acid. In some embodiments, a 2A peptide sequence is a P2A (derived from porcine teschovirus-1 2A), E2A (derived from equine rhinitis A virus), F2A (derived from foot-and-mouth disease virus), or T2A (derived from Thosea asigna virus 2A) peptide sequence. A T2A peptide sequence may comprise, for example, the amino acid sequence of EGRGSLLTCGDVEENPGP (SEQ ID NO: 102). A P2A peptide sequence may comprise, for example, the amino acid sequence of ATNFSLLKQAGDVEENPGP (SEQ ID NO: 103). An E2A peptide sequence may comprise, for example, the amino acid sequence of QCTNYALLKLAGDVESNPGP (SEQ ID NO: 104). A F2A peptide sequence may comprise, for example, the amino acid sequence of VKQTLNFDLLKLAGDVESNPGP (SEQ ID NO: 105).

In some embodiments, a TCR comprises two or more polypeptides. For example, in some embodiments, a TCR comprises a first polypeptide comprising an alpha chain and a second polypeptide comprising a beta chain.

In some embodiments, a TCR comprises one or more cysteine residues present in the alpha chain of the TCR that are capable of forming one or more disulfide bonds with one or more cysteine residues in the beta chain of the TCR. In some embodiments, an exogenous TCR comprises one or more cysteine residues present in the alpha chain constant region of the TCR that are capable of forming one or more disulfide bonds with one or more cysteine residues in the beta chain constant region of the TCR.

In some embodiments, the TCR alpha chain constant region comprises an amino acid substitution at position 48 to introduce a cysteine (e.g., T48C) relative to a TCR alpha chain constant region comprising the amino acid sequence of SEQ ID NO: 106. In some embodiments, the TCR beta chain constant region comprises an amino acid substitution at position 57 to introduce a cysteine (e.g., S57C) amino acid substitution relative to a TCR beta chain constant region comprising the amino acid sequence of SEQ ID NO: 108. In some embodiments, the TCR alpha chain constant region comprises an amino acid substitution at position 48 to introduce a cysteine (e.g., T48C) relative to a TCR alpha chain constant region comprising the amino acid sequence of SEQ ID NO: 106, and the TCR beta chain constant region comprises an amino acid substitution at position 57 to introduce a cysteine (e.g., S57C) amino acid substitution relative to a TCR beta chain constant region comprising the amino acid sequence of SEQ ID NO: 108, wherein the cysteine residue at position 48 of the alpha chain is capable of forming a disulfide bond with the cysteine residue at position 57 of the beta chain.

In some embodiments, the TCR alpha chain constant region comprises an amino acid substitution at position 45 to introduce a cysteine (e.g., T45C) relative to a TCR alpha chain constant region comprising the amino acid sequence of SEQ ID NO: 106. In some embodiments, the TCR beta chain constant region comprises an amino acid substitution at position 77 to introduce a cysteine (e.g., S77C) amino acid substitution relative to a TCR beta chain constant region comprising the amino acid sequence of SEQ ID NO: 108. In some embodiments, the TCR alpha chain constant region comprises an amino acid substitution at position 45 to introduce a cysteine (e.g., T45C) relative to a TCR alpha chain constant region comprising the amino acid sequence of SEQ ID NO: 106, and the TCR beta chain constant region comprises an amino acid substitution at position 77 to introduce a cysteine (e.g., S77C) amino acid substitution relative to a TCR beta chain constant region comprising the amino acid sequence of SEQ ID NO: 108, wherein the cysteine residue at position 45 of the alpha chain is capable of forming a disulfide bond with the cysteine residue at position 77 of the beta chain.

In some embodiments, the TCR alpha chain constant region comprises an amino acid substitution at position 10 to introduce a cysteine (e.g., Y10C) relative to a TCR alpha chain constant region comprising the amino acid sequence of SEQ ID NO: 106. In some embodiments, the TCR beta chain constant region comprises an amino acid substitution at position 17 to introduce a cysteine (e.g., S17C) amino acid substitution relative to a TCR beta chain constant region comprising the amino acid sequence of SEQ ID NO: 108. In some embodiments, the TCR alpha chain constant region comprises an amino acid substitution at position 10 to introduce a cysteine (e.g., Y10C) relative to a TCR alpha chain constant region comprising the amino acid sequence of SEQ ID NO: 106, and the TCR beta chain constant region comprises an amino acid substitution at position 17 to introduce a cysteine (e.g., S17C) amino acid substitution relative to a TCR beta chain constant region comprising the amino acid sequence of SEQ ID NO: 108, wherein the cysteine residue at position 10 of the alpha chain is capable of forming a disulfide bond with the cysteine residue at position 17 of the beta chain.

In some embodiments, the TCR alpha chain constant region comprises an amino acid substitution at position 45 to introduce a cysteine (e.g., T45C) relative to a TCR alpha chain constant region comprising the amino acid sequence of SEQ ID NO: 106. In some embodiments, the TCR beta chain constant region comprises an amino acid substitution at position 59 to introduce a cysteine (e.g., D59C) amino acid substitution relative to a TCR beta chain constant region comprising the amino acid sequence of SEQ ID NO: 108. In some embodiments, the TCR alpha chain constant region comprises an amino acid substitution at position 45 to introduce a cysteine (e.g., T45C) relative to a TCR alpha chain constant region comprising the amino acid sequence of SEQ ID NO: 106, and the TCR beta chain constant region comprises an amino acid substitution at position 59 to introduce a cysteine (e.g., D59C) amino acid substitution relative to a TCR beta chain constant region comprising the amino acid sequence of SEQ ID NO: 108, wherein the cysteine residue at position 45 of the alpha chain is capable of forming a disulfide bond with the cysteine residue at position 59 of the beta chain.

In some embodiments, the TCR alpha chain constant region comprises an amino acid substitution at position 15 to introduce a cysteine (e.g., S15C) relative to a TCR alpha chain constant region comprising the amino acid sequence of SEQ ID NO: 106. In some embodiments, the TCR beta chain constant region comprises an amino acid substitution at position 15 to introduce a cysteine (e.g., E15C) amino acid substitution relative to a TCR beta chain constant region comprising the amino acid sequence of SEQ ID NO: 108. In some embodiments, the TCR alpha chain constant region comprises an amino acid substitution at position 15 to introduce a cysteine (e.g., S15C) relative to a TCR alpha chain constant region comprising the amino acid sequence of SEQ ID NO: 106, and the TCR beta chain constant region comprises an amino acid substitution at position 15 to introduce a cysteine (e.g., E15C) amino acid substitution relative to a TCR beta chain constant region comprising the amino acid sequence of SEQ ID NO: 108, wherein the cysteine residue at position 15 of the alpha chain is capable of forming a disulfide bond with the cysteine residue at position 15 of the beta chain.

### Nucleic Acids Encoding a TCR

In some embodiments, the disclosure provides nucleic acids encoding a TCR (e.g., a TCR as described in Table 1). Nucleic acids may be or may include deoxyribonucleic acid (DNA), ribonucleic acid (RNA) (e.g., messenger RNA), threose nucleic acid (TNA), glycol nucleic acid (GNA), peptide nucleic acid (PNA), locked nucleic acid (LNA), ethylene nucleic acid (ENA), cyclohexenyl nucleic acid (CeNA) and/or chimeras.

The nucleic acids used herein are generally engineered nucleic acids. An engineered nucleic acid is a polynucleotide (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. The MegaGate molecular cloning method may also be used. MegaGate is a toxin-less Gateway technology that eliminates the ccdb toxin used in Gateway recombinase cloning and instead utilizes meganuclease-mediated digestion to eliminate background vectors during cloning (see, e.g., Kramme C. et al. STAR Protoc. 2021 Oct. 22; 2 (4): 100907, incorporated herein by reference). Other methods of producing engineered polynucleotides may be used in accordance with the present disclosure.

In some embodiments, an engineered nucleic acid comprises a promoter operably linked to an open reading frame. A promoter is a nucleotide sequence to which RNA polymerase binds to initial transcription (e.g., ATG). Promoters are typically located directly upstream from (at the 5′ end of) a transcription initiation site. In some embodiments, a promoter is a heterologous promoter. A heterologous promoter is not naturally associated with the open reading frame to which is it operably linked. In some embodiments, a promoter is an inducible promoter. An inducible promoter may be regulated in vivo by a chemical agent, temperature, or light, for example. Non-limiting examples of promoters that may be used as provided herein include the eukaryotic translation elongation factor 1 alpha (EF-1 alpha) promoter and the MND promoter (myeloproliferative sarcoma virus enhancer, negative control region deleted, d1587rev primer-binding site substituted) (see, e.g., Gill, D R. et al. Gene Ther. 2001; 8:1539-46 and Astrakhan, A. et al. Blood 2012; 119:4395-4407).

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

A nucleic acid encoding a TCR may be a vector or plasmid. In some embodiments, the vector is a viral vector. For example, the vector may be a lentiviral vector, an adenovirus vector, an adeno-associated viral (AAV) vector, a herpes viral vector, a retroviral vector, or a baculoviral vector. A viral vector provides efficient delivery of the exogenous TCR into regulatory T cells of the disclosure. Exemplary viral vectors may be derived from lentivirus, retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, alpharetrovirus, gammaretrovirus, spumavirus, murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses.

In some embodiments, a nucleic acid encoding an exogenous TCR is an RNA (e.g., a messenger RNA (mRNA)). In some embodiments, an mRNA comprises a 5′ cap, a 5′ untranslated region (UTR), an ORF, a 3′ UTR, and/or a poly(A) tail.

In some embodiments, a nucleic acid is codon optimized. Codon optimization methods are known in the art. Codon optimization, in some embodiments, may be used to match codon frequencies in target and host organisms to ensure proper folding; bias GC content to increase RNA (e.g., mRNA) stability or reduce secondary structures; minimize tandem repeat codons or base runs that may impair gene construction or expression; customize transcriptional and translational control regions; insert or remove protein trafficking sequences; remove/add post-translation modification sites in encoded protein (e.g., glycosylation sites); add, remove or shuffle protein domains; insert or delete restriction sites; modify ribosome binding sites and RNA (e.g., mRNA) degradation sites; adjust translational rates to allow the various domains of the protein to fold properly; or reduce or eliminate problem secondary structures within the polynucleotide.

In some embodiments, a nucleic acid encoding a TCR comprises a promoter operably linked to a coding sequence encoding the exogenous human TCR. A promoter may be a viral promoter or a natural TCR promoter. In some embodiments, a promoter is a constitutively active promoter or an inducible promoter. In some embodiments, a promoter is the eukaryotic translation elongation factor 1 alpha (EF-1 alpha) promoter and the MND promoter (myeloproliferative sarcoma virus enhancer, negative control region deleted, d1587rev primer-binding site substituted) (see, e.g., Gill, D R. et al. Gene Ther. 2001; 8:1539-46 and Astrakhan, A. et al. Blood 2012; 119:4395-4407).

A vector may also include a termination codon and/or expression enhancer elements. Any suitable vectors, promoters, enhancers and termination codons known in the art may be used. In some embodiments, an enhancer element is an optimized post-transcriptional regulatory element (oPRE), a woodchuck hepatitis virus post-transcriptional regulatory element (WPRE). A WPRE may be a wild-type WPRE or a WPRE mutant sequence (e.g., WPRE-mut6). In some embodiments, a WPRE is as described in Zanta-Boussif, M. A. et al., Gene Therapy volume 16, pages 605-619 (2009).

In some embodiments, a vector encoding a T cell receptor (TCR) is a recombinant lentiviral vector encoding the TCR. A recombinant lentiviral vector may be a third generation Vesicular Stomatitis Virus glycoprotein (VSV-G) pseudotyped, self-inactivating vector (e.g., that was generated using a split-genome four plasmid system (rev; gag+pol; VSV-G; gene encoding the TCR)). In some embodiments, a recombinant lentiviral vector encodes any of the TCRs described herein. In some embodiments, a recombinant lentiviral vector encodes a TCR that comprises one or more amino acid sequences set forth in Table 1. In some embodiments, a recombinant lentiviral vector encodes the B and a chains of any one of the TCRs provided in Table 1, wherein the B and a chains are linked by a ribosome skip P2A element.

Isolated Cell Regulatory T cells

The present disclosure provides methods of producing regulatory T cells (e.g., stable regulatory T cells) that express a TCR described herein and compositions comprising such regulatory T cells. These regulatory T cells may be used, for example, to treat Type 1 Diabetes. A regulatory T cell, also referred to as a “Treg”, is a T cell that modulates the immune system. Regulatory T cells are immunosuppressive and generally suppress or downregulate induction and proliferation of effector T cells. Regulatory T cells are thought to be derived from the same lineage as naïve CD4+ cells and express the biomarkers CD4, CD25, and FOXP3. In some embodiments, the regulatory T cells (e.g., stable regulatory T cells) comprise an exogenous human T cell receptor (TCR) that binds specifically to GAD65. This binding occurs when the GAD65 is complexed with a major histocompatibility complex (MHC) molecule (e.g., MHC Class I or MHC Class II).

A stable, or thymic, regulatory T cell is a regulatory T cell that comprises a hypomethylated regulatory T cell-specific demethylation region (TSDR) at an endogenous FOXP3 locus and expresses CD4, CD25, and FOXP3. A regulatory T cell is considered “stable” if it is terminally differentiated, i.e., it has lost its ability to change its cell fate. In thymic regulatory T cell development, the genome organizer SATB1 (special AT-rich sequence-binding protein) binds to specific genomic sites from the CD4⁺CD8⁺ thymocyte stage to open up the chromatin and activate super-enhancers associated with many regulatory T cell signature genes such as FOXP3, IL2RA, (CD25), CTLA4, IKZF2 (HELIOS), and IFZF4 (EOS). SATB1 and MLL4 (myeloid/lymphoid or mixed-lineage leukemia 4), an enzyme involved in enhancer priming, commonly occupy the newly identified conserved enhancer region, designated conserved noncoding sequence 0 (CNS0), at the FOXP3 locus, with subsequent activation of the enhancers at CNS3 and CNS2, and then the promoter. This results in stable hypomethylation and expression of FOXP3 and other regulator T cell-associated genes, thereby resulting in a stable regulatory T cell phenotype. Epigenetic mechanisms, in particular, DNA methylation/demethylation, which is heritable through cell divisions, play an essential role for stable maintenance of regulatory T cell-specific gene expression. Hypomethylation of the TSDR, which is an evolutionary conserved CpG-rich regulatory element of the FOXP3 gene, is associated with expression of FOXP3. A TSDR of an endogenous FOXP3 locus is hypomethylated when the methyl group from one or more methylated cytosines in the TSDR have been removed to replace the methylated cytosine(s) with cytosine. In some embodiments, measurement of the methylation status of the TSDR of a FOXP3 locus is as described in Kressler et. Al. “Targeted De-Methylation of the FOXP3-TSDR Is Sufficient to Induce Physiological FOXP3 Expression but Not a Functional Regulatory T Phenotype” Frontiers in Immunology, 7 Jan. 2021.; or Schreiber, et. Al. “The Regulatory T-Specific Demethylated Region Stabilizes Foxp3 Expression Independently of NF-κB Signaling” PLOS One, Feb. 5, 2014. For example, genomic DNA may be isolated and bisulfite conversion performed. This converts, in some embodiments, all the unmethylated cysteines to uracils. The TSDR region can be amplified using primers that amplify the bisulfite-converted DNA, independent of methylation status. Two competing probes detect the abundance of methylated or unmethylated CpG, with the total and/or relative abundance being determined by ddPCR or pyrosequencing. Alternatively, next-generation sequencing (NGS) can be performed on the bisulfite-converted DNA to determine the sequence of the bisulfite-converted DNA at the TSDR locus.

In some embodiments, a stable regulatory T cell retains markers of stability in the presence of pro-inflammatory conditions (e.g., in presence of one or more pro-inflammatory cytokines). In some embodiments, a stable regulatory T cell comprises a hypomethylated regulatory T cell-specific demethylation region (TSDR) at an endogenous FOXP3 locus and expresses CD4, CD25, and FOXP3 in the presence of pro-inflammatory conditions (e.g., in presence of one or more pro-inflammatory cytokines). In some embodiments, a stable regulatory T cell comprises a hypomethylated regulatory T cell-specific demethylation region (TSDR) at an endogenous FOXP3 locus and expresses CD4, CD25, and FOXP3 in the presence of pro-inflammatory conditions for at least 3, 4, 5, 6, 7, 8, 9, 10, 11, 12, 13, 14, 15, 20, 21, 22, 23, 24, or 25 days.

A stable regulatory T cell that expresses an exogenous TCR (e.g., a TCR as described in Table 1) that binds specifically to a target peptide (e.g., GAD65) complexed with an MHC molecule further exhibits one or more of the following functions: (i) cytokine secretion activity (e.g., secretion of IL-10, IL-4, IL-6, IL-11, and/or IL-13) when the TCR is contacted with its target peptide; (ii) expression of activation markers associated with regulatory T cells (e.g., expression of CD69, 4-1BB, CD25, CD71, and/or CTLA-4) when the TCR is contacted with its target peptide; and/or (iii) suppression activity (e.g., the ability of the stable regulatory T cell to suppress the activation of conventional T cells having specificity towards a shared target peptide) when the TCR is contacted with the target peptide complexed with an MHC molecule.

Thus, regulatory T cells of the isolated cell populations provided herein may comprise a hypomethylated TSDR at an endogenous FOXP3 locus, expression of CD4, CD25, and FOXP3, and/or exhibit cytokine secretion, activation, and/or suppression activity.

In some embodiments, a regulatory T cell (e.g., a stable regulatory T cell) is CD25⁺. In some embodiments, a regulatory T cell (e.g., a stable regulatory T cell) is CD25^{high}. As is used herein, “CD25^{+/high}” means that the cell is either CD25⁺ or CD25^{high}. A CD25⁺ cell is a cell that expresses a detectable level of CD25 (e.g., detectable by FACS). A CD25^{+/high} cell is a cell that expresses CD25 above a threshold or control level. For example, in the context of an isolated cell population that comprises CD25⁺ cells, a subpopulation of those cells may express higher levels of CD25 relative to another subpopulation of CD25⁺ cells in that population (or relative to all the other cells CD25+ cells in the population). The former subpopulation would be considered to be CD25^{high}, while the latter subpopulation would be considered only CD25⁺ (not CD25^{high}).

In some embodiments, a regulatory T cell (e.g., a stable regulatory T cell) is CD4⁺.

In some embodiments, a regulatory T cell (e.g., a stable regulatory T cell) is CD127^{-/lo}. As is used herein, “CD127^{-/lo}” means that the cell is either CD127⁻ or CD127^{lo}. A CD127⁻ cell is a cell that does not express CD127, does not express a detectable level of CD127 (e.g., by FACS), or expresses CD127 below a threshold or control level. For example, in the context of an isolated cell population that comprises at least some proportion of CD127⁺ cells (e.g., detectable by FACS), a subpopulation of those cells may express lower levels of CD127 relative to another subpopulation of CD127⁺ cells in that population (or relative to all the other cells CD127+ cells in the population). The former subpopulation would be considered to be CD1271^{lo} (not CD127⁻).

In some embodiments, a regulatory T cell (e.g., a stable regulatory T cell) is CD45RA⁺.

In some embodiments, a regulatory T cell (e.g., a stable regulatory T cell) is FOXP3⁺.

In some embodiments, a regulatory T cell (e.g., a stable regulatory T cell) is a CD25^{+/high}CD4⁺CD127^{-/lo} cell. That is, the regulatory T cell expresses, or expresses a high level of CD25, expresses CD4, and does not express, or expresses a low level of, CD127. In some embodiments, a regulatory T cell does not express CD127. In some embodiments, a regulatory T cell (e.g., a stable regulatory T cell) is a CD25^{+/high}CD4⁺CD127^{-/lo}/CD45RA⁺ cell. Thus, the regulatory T cells expresses, or expresses a high level of CD25, expresses CD4 and CD45RA, and does not express, or expresses a low level of, CD127. In some embodiments, a regulatory T cell expresses FOXP3. In some embodiments, a regulatory T cell is a CD25^{+/high}CD4⁺CD127^{-/lo} FOXP3⁺ cell. Thus, the regulatory T cells expresses, or expresses a high level of CD25, expresses CD4 and FOXP3, and does not express, or expresses a low level of, CD127. In some embodiments, a regulatory T cell is a CD25^{+/high}CD4⁺CD45RA⁺CD127^{-/lo}FOXP3⁺ cell. Thus, the regulatory T cells expresses, or expresses a high level of CD25, expresses CD4, CD45RA and FOXP3, and do not express, or expresses a low level of CD127.

A cell “expresses” a biomarker (e.g., CD4, CD25, CD45RA, and/or FOXP3) if the biomarker can be detected using a conventional protein expression assay, such as an antibody detection assay. An antibody detection assay in this context involves the detection (e.g., using fluorescence activated cell sorting (FACS) or Western blot) of an antibody that binds to the biomarker of interest (e.g., CD4, CD25, CD45RA, or FOXP3) when the biomarker is present within a cell or on the surface of a cell. A cell “does not express” a biomarker (e.g., CD127) if the expression of the biomarker cannot be detected using a conventional protein assay. A cell “expresses a low level” of a biomarker (e.g., CD127), if expression of that biomarker is lower than (e.g., at least 50%, at least 60%, at least 70%, at least 80%, or at least 90% lower than) a control level. In some embodiments, the control level of a biomarker in a regulatory T cell is the expression level of the biomarker (e.g., CD127) in a conventional T cell or a CD8⁺ T cell. For example, a regulatory T cell expresses a low level of CD127 if its expression of CD127 is lower than (e.g., at least 50%, at least 60%, at least 70%, at least 80%, or at least 90% lower than) expression of CD127 in a conventional T cell.

In some embodiments, a stable regulatory T cell of an isolated cell population provided herein maintains a hypomethylated TSDR at an endogenous FOXP3 locus over time (i.e., the TSDR at the endogenous FOXP3 locus of viable cells (e.g., in culture, in a cryoprotective agent, and/or in vivo) is hypomethylated for a certain measurable period of time). For example, a stable regulatory T cell of an isolated cell population may maintain a hypomethylated TSDR at an endogenous FOXP3 locus for at least 1, 2, 3, 4, 5, 6, 7, 8, 9, 10, 11, 12, 13, 14, 15, 16, 17, 18, 19, or 20 days (e.g., in culture, in a cryoprotective agent, and/or in vivo after being obtained from a subject or following transduction with a nucleic acid expressing a TCR). In some embodiments, a stable regulatory T cell maintains a hypomethylated TSDR at an endogenous FOXP3 locus for more than 5 days, more than 10 days, more than 15 days, or more than 20 days (e.g., in culture, in a cryoprotective agent, and/or in vivo after being obtained from a subject or following transduction with a nucleic acid expressing a TCR). In some embodiments, a stable regulatory T cell maintains a hypomethylated TSDR at an endogenous FOXP3 locus for 1-20 days, 1-10 days, 1-5 days, 5-30 days, 5-20 days, 10-40 days, or 25-50 days (e.g., in culture, in a cryoprotective agent, and/or in vivo after being obtained from a subject or following transduction with a nucleic acid expressing a TCR).

A regulatory T cell of an isolated cell population of the disclosure, in some embodiments, is an autologous cell. The term autologous in this context refers to cells that have been obtained from the same subject to which they are subsequently administered (e.g., following engineering to introduce an exogenous TCR into the cells). For example, a population of cells may be obtained from a subject, subjected to the methods described herein, and then administered to the same subject (from which the population of cells was originally obtained) to treat Type 1 Diabetes. In such embodiments, the population of cells administered to the subject comprise autologous regulatory T cells.

A regulatory T cell of the disclosure, in some embodiments, is an allogenic cell. The term allogenic in this context refers to cells that have been obtained from one subject and then administered to another subject. For example, a population of cells may be obtained from a subject, subjected to the methods described herein, and then administered to another subject in order to treat an autoimmune disease.

In some embodiments, regulatory T cells (e.g., a biological sample comprising regulatory T cells) are obtained from a subject diagnosed with, or suspected of having, Type 1 Diabetes.

Some aspects relate to an engineered regulatory T cell that comprises an exogenous TCR that binds to a GAD65 peptide complexed with a major histocompatibility complex (MHC), wherein the TCR comprises an alpha chain and a beta chain, and wherein the TCR comprises: a CDR1-alpha sequence comprising the amino acid sequence of SEQ ID NO: 1, a CDR2-alpha sequence comprising the amino acid sequence of SEQ ID NO: 2, a CDR3-alpha sequence comprising the amino acid sequence of SEQ ID NO: 3, a CDR1-beta sequence comprising the amino acid sequence of SEQ ID NO: 6, a CDR2-beta sequence comprising the amino acid sequence of SEQ ID NO: 7, and/or a CDR3-beta sequence comprising the amino acid sequence of SEQ ID NO: 8.

In some embodiments, the TCR comprises a CDR1-alpha sequence comprising the amino acid sequence of SEQ ID NO: 1, a CDR2-alpha sequence comprising the amino acid sequence of SEQ ID NO: 2, and a CDR3-alpha sequence comprising the amino acid sequence of SEQ ID NO: 3. In some embodiments, the alpha chain comprises an alpha variable region comprising an amino acid sequence having at least 90%, 95%, or 100% identity to the amino acid sequence of SEQ ID NO: 4. In some embodiments, the alpha chain comprises an amino acid sequence having at least 90%, 95%, or 100% identity to the amino acid sequence of SEQ ID NO: 5. In some embodiments, the alpha chain comprises an amino acid sequence having at least 90%, 95%, or 100% identity to the amino acid sequence of SEQ ID NO: 260.

In some embodiments, the TCR comprises a CDR1-beta sequence comprising the amino acid sequence of SEQ ID NO: 6, a CDR2-beta sequence comprising the amino acid sequence of SEQ ID NO: 7, and a CDR3-beta sequence comprising the amino acid sequence of SEQ ID NO: 8. In some embodiments, the beta chain comprises a beta variable region comprising an amino acid sequence having at least 90%, 95%, or 100% identity to the amino acid sequence of SEQ ID NO: 9. In some embodiments, the beta chain comprises an amino acid sequence having at least 90%, 95%, or 100% identity to the amino acid sequence of SEQ ID NO: 10. In some embodiments, the beta chain comprises an amino acid sequence having at least 90%, 95%, or 100% identity to the amino acid sequence of SEQ ID NO: 261.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 11, a CDR2-alpha sequence comprising the amino acid sequence of SEQ ID NO: 12, a CDR3-alpha sequence comprising the amino acid sequence of SEQ ID NO: 13, a CDR1-beta sequence comprising the amino acid sequence of SEQ ID NO: 16, a CDR2-beta sequence comprising the amino acid sequence of SEQ ID NO: 17, and/or a CDR3-beta sequence comprising the amino acid sequence of SEQ ID NO: 18.

In some embodiments, the TCR comprises a CDR1-alpha sequence comprising the amino acid sequence of SEQ ID NO: 11, a CDR2-alpha sequence comprising the amino acid sequence of SEQ ID NO: 12, and a CDR3-alpha sequence comprising the amino acid sequence of SEQ ID NO: 13. In some embodiments, the alpha chain comprises an alpha variable region comprising an amino acid sequence having at least 90%, 95%, or 100% identity to the amino acid sequence of SEQ ID NO: 14. In some embodiments, the alpha chain comprises an amino acid sequence having at least 90%, 95%, or 100% identity to the amino acid sequence of SEQ ID NO: 15. In some embodiments, the alpha chain comprises an amino acid sequence having at least 90%, 95%, or 100% identity to the amino acid sequence of SEQ ID NO: 262.

In some embodiments, the TCR comprises a CDR1-beta sequence comprising the amino acid sequence of SEQ ID NO: 16, a CDR2-beta sequence comprising the amino acid sequence of SEQ ID NO: 17, and a CDR3-beta sequence comprising the amino acid sequence of SEQ ID NO: 18. In some embodiments, the beta chain comprises an alpha variable region comprising an amino acid sequence having at least 90%, 95%, or 100% identity to the amino acid sequence of SEQ ID NO: 19. In some embodiments, the beta chain comprises an amino acid sequence having at least 90%, 95%, or 100% identity to the amino acid sequence of SEQ ID NO: 20. In some embodiments, the beta chain comprises an amino acid sequence having at least 90%, 95%, or 100% identity to the amino acid sequence of SEQ ID NO: 263.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 21, a CDR2-alpha sequence comprising the amino acid sequence of SEQ ID NO: 22, a CDR3-alpha sequence comprising the amino acid sequence of SEQ ID NO: 23, a CDR1-beta sequence comprising the amino acid sequence of SEQ ID NO: 26, a CDR2-beta sequence comprising the amino acid sequence of SEQ ID NO: 27, and/or a CDR3-beta sequence comprising the amino acid sequence of SEQ ID NO: 28.

In some embodiments, the TCR comprises a CDR1-alpha sequence comprising the amino acid sequence of SEQ ID NO: 21, a CDR2-alpha sequence comprising the amino acid sequence of SEQ ID NO: 22, and a CDR3-alpha sequence comprising the amino acid sequence of SEQ ID NO: 23. In some embodiments, the alpha chain comprises an alpha variable region comprising an amino acid sequence having at least 90%, 95%, or 100% identity to the amino acid sequence of SEQ ID NO: 24. In some embodiments, the alpha chain comprises an amino acid sequence having at least 90%, 95%, or 100% identity to the amino acid sequence of SEQ ID NO: 25. In some embodiments, the alpha chain comprises an amino acid sequence having at least 90%, 95%, or 100% identity to the amino acid sequence of SEQ ID NO: 264.

In some embodiments, the TCR comprises a CDR1-beta sequence comprising the amino acid sequence of SEQ ID NO: 26, a CDR2-beta sequence comprising the amino acid sequence of SEQ ID NO: 27, and a CDR3-beta sequence comprising the amino acid sequence of SEQ ID NO: 28. In some embodiments, the beta chain comprises a beta variable region comprising an amino acid sequence having at least 90%, 95%, or 100% identity to the amino acid sequence of SEQ ID NO: 29. In some embodiments, the beta chain comprises an amino acid sequence having at least 90%, 95%, or 100% identity to the amino acid sequence of SEQ ID NO: 30. In some embodiments, the beta chain comprises an amino acid sequence having at least 90%, 95%, or 100% identity to the amino acid sequence of SEQ ID NO: 265.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises a CDR1-alpha sequence comprising the amino acid sequence of SEQ ID NO: 31, a CDR2-alpha sequence comprising the amino acid sequence of SEQ ID NO: 32, a CDR3-alpha sequence comprising the amino acid sequence of SEQ ID NO: 33, a CDR1-beta sequence comprising the amino acid sequence of SEQ ID NO: 36, a CDR2-beta sequence comprising the amino acid sequence of SEQ ID NO: 37, and/or a CDR3-beta sequence comprising the amino acid sequence of SEQ ID NO: 38.

In some embodiments, the TCR comprises a CDR1-alpha sequence comprising the amino acid sequence of SEQ ID NO: 31, a CDR2-alpha sequence comprising the amino acid sequence of SEQ ID NO: 32, and a CDR3-alpha sequence comprising the amino acid sequence of SEQ ID NO: 33. In some embodiments, the alpha chain comprises an alpha variable region comprising an amino acid sequence having at least 90%, 95%, or 100% identity to the amino acid sequence of SEQ ID NO: 34. In some embodiments, the alpha chain comprises an amino acid sequence having at least 90%, 95%, or 100% identity to the amino acid sequence of SEQ ID NO: 35. In some embodiments, the alpha chain comprises an amino acid sequence having at least 90%, 95%, or 100% identity to the amino acid sequence of SEQ ID NO: 266.

In some embodiments, the TCR comprises a CDR1-beta sequence comprising the amino acid sequence of SEQ ID NO: 36, a CDR2-beta sequence comprising the amino acid sequence of SEQ ID NO: 37, and a CDR3-beta sequence comprising the amino acid sequence of SEQ ID NO: 38. In some embodiments, the beta chain comprises a beta variable region comprising an amino acid sequence having at least 90%, 95%, or 100% identity to the amino acid sequence of SEQ ID NO: 39. In some embodiments, the beta chain comprises an amino acid sequence having at least 90%, 95%, or 100% identity to the amino acid sequence of SEQ ID NO: 40. In some embodiments, the beta chain comprises an amino acid sequence having at least 90%, 95%, or 100% identity to the amino acid sequence of SEQ ID NO: 267.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 41, a CDR2-alpha sequence comprising the amino acid sequence of SEQ ID NO: 42, a CDR3-alpha sequence comprising the amino acid sequence of SEQ ID NO: 43, a CDR1-beta sequence comprising the amino acid sequence of SEQ ID NO: 46, a CDR2-beta sequence comprising the amino acid sequence of SEQ ID NO: 47, and/or a CDR3-beta sequence comprising the amino acid sequence of SEQ ID NO: 48.

In some embodiments, the TCR comprises a CDR1-alpha sequence comprising the amino acid sequence of SEQ ID NO: 41, a CDR2-alpha sequence comprising the amino acid sequence of SEQ ID NO: 42, and a CDR3-alpha sequence comprising the amino acid sequence of SEQ ID NO: 43. In some embodiments, the alpha chain comprises an alpha variable region comprising an amino acid sequence having at least 90%, 95%, or 100% identity to the amino acid sequence of SEQ ID NO: 44. In some embodiments, the alpha chain comprises an amino acid sequence having at least 90%, 95%, or 100% identity to the amino acid sequence of SEQ ID NO: 45. In some embodiments, the alpha chain comprises an amino acid sequence having at least 90%, 95%, or 100% identity to the amino acid sequence of SEQ ID NO: 268.

In some embodiments, the TCR comprises a CDR1-beta sequence comprising the amino acid sequence of SEQ ID NO: 46, a CDR2-beta sequence comprising the amino acid sequence of SEQ ID NO: 47, and a CDR3-beta sequence comprising the amino acid sequence of SEQ ID NO: 48. In some embodiments, the beta chain comprises a beta variable region comprising an amino acid sequence having at least 90%, 95%, or 100% identity to the amino acid sequence of SEQ ID NO: 49. In some embodiments, the beta chain comprises an amino acid sequence having at least 90%, 95%, or 100% identity to the amino acid sequence of SEQ ID NO: 50. In some embodiments, the beta chain comprises an amino acid sequence having at least 90%, 95%, or 100% identity to the amino acid sequence of SEQ ID NO: 269.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 51, a CDR2-alpha sequence comprising the amino acid sequence of SEQ ID NO: 52, a CDR3-alpha sequence comprising the amino acid sequence of SEQ ID NO: 53, a CDR1-beta sequence comprising the amino acid sequence of SEQ ID NO: 56, a CDR2-beta sequence comprising the amino acid sequence of SEQ ID NO: 57, and/or a CDR3-beta sequence comprising the amino acid sequence of SEQ ID NO: 58.

In some embodiments, the TCR comprises a CDR1-alpha sequence comprising the amino acid sequence of SEQ ID NO: 51, a CDR2-alpha sequence comprising the amino acid sequence of SEQ ID NO: 52, and a CDR3-alpha sequence comprising the amino acid sequence of SEQ ID NO: 53. In some embodiments, the alpha chain comprises an alpha variable region comprising an amino acid sequence having at least 90%, 95%, or 100% identity to the amino acid sequence of SEQ ID NO: 54. In some embodiments, the alpha chain comprises an amino acid sequence having at least 90%, 95%, or 100% identity to the amino acid sequence of SEQ ID NO: 55. In some embodiments, the alpha chain comprises an amino acid sequence having at least 90%, 95%, or 100% identity to the amino acid sequence of SEQ ID NO: 270.

In some embodiments, the TCR comprises a CDR1-beta sequence comprising the amino acid sequence of SEQ ID NO: 56, a CDR2-beta sequence comprising the amino acid sequence of SEQ ID NO: 57, and a CDR3-beta sequence comprising the amino acid sequence of SEQ ID NO: 58. In some embodiments, the beta chain comprises a beta variable region comprising an amino acid sequence having at least 90%, 95%, or 100% identity to the amino acid sequence of SEQ ID NO: 59. In some embodiments, the beta chain comprises an amino acid sequence having at least 90%, 95%, or 100% identity to the amino acid sequence of SEQ ID NO: 60. In some embodiments, the beta chain comprises an amino acid sequence having at least 90%, 95%, or 100% identity to the amino acid sequence of SEQ ID NO: 271.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 61, a CDR2-alpha sequence comprising the amino acid sequence of SEQ ID NO: 62, a CDR3-alpha sequence comprising the amino acid sequence of SEQ ID NO: 63, a CDR1-beta sequence comprising the amino acid sequence of SEQ ID NO: 66, a CDR2-beta sequence comprising the amino acid sequence of SEQ ID NO: 67, and/or a CDR3-beta sequence comprising the amino acid sequence of SEQ ID NO: 68.

In some embodiments, the TCR comprises a CDR1-alpha sequence comprising the amino acid sequence of SEQ ID NO: 61, a CDR2-alpha sequence comprising the amino acid sequence of SEQ ID NO: 62, and a CDR3-alpha sequence comprising the amino acid sequence of SEQ ID NO: 63. In some embodiments, the alpha chain comprises an alpha variable region comprising an amino acid sequence having at least 90%, 95%, or 100% identity to the amino acid sequence of SEQ ID NO: 64. In some embodiments, the alpha chain comprises an amino acid sequence having at least 90%, 95%, or 100% identity to the amino acid sequence of SEQ ID NO: 65. In some embodiments, the alpha chain comprises an amino acid sequence having at least 90%, 95%, or 100% identity to the amino acid sequence of SEQ ID NO: 272.

In some embodiments, the TCR comprises a CDR1-beta sequence comprising the amino acid sequence of SEQ ID NO: 66, a CDR2-beta sequence comprising the amino acid sequence of SEQ ID NO: 67, and a CDR3-beta sequence comprising the amino acid sequence of SEQ ID NO: 68. In some embodiments, the beta chain comprises a beta variable region comprising an amino acid sequence having at least 90%, 95%, or 100% identity to the amino acid sequence of SEQ ID NO: 69. In some embodiments, the beta chain comprises an amino acid sequence having at least 90%, 95%, or 100% identity to the amino acid sequence of SEQ ID NO: 70. In some embodiments, the beta chain comprises an amino acid sequence having at least 90%, 95%, or 100% identity to the amino acid sequence of SEQ ID NO: 273.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 71, a CDR2-alpha sequence comprising the amino acid sequence of SEQ ID NO: 72, a CDR3-alpha sequence comprising the amino acid sequence of SEQ ID NO: 73, a CDR1-beta sequence comprising the amino acid sequence of SEQ ID NO: 76, a CDR2-beta sequence comprising the amino acid sequence of SEQ ID NO: 77, and/or a CDR3-beta sequence comprising the amino acid sequence of SEQ ID NO: 78.

In some embodiments, the TCR comprises a CDR1-alpha sequence comprising the amino acid sequence of SEQ ID NO: 71, a CDR2-alpha sequence comprising the amino acid sequence of SEQ ID NO: 72, and a CDR3-alpha sequence comprising the amino acid sequence of SEQ ID NO: 73. In some embodiments, the alpha chain comprises an alpha variable region comprising an amino acid sequence having at least 90%, 95%, or 100% identity to the amino acid sequence of SEQ ID NO: 74. In some embodiments, the alpha chain comprises an amino acid sequence having at least 90%, 95%, or 100% identity to the amino acid sequence of SEQ ID NO: 75. In some embodiments, the alpha chain comprises an amino acid sequence having at least 90%, 95%, or 100% identity to the amino acid sequence of SEQ ID NO: 274.

In some embodiments, the TCR comprises a CDR1-beta sequence comprising the amino acid sequence of SEQ ID NO: 76, a CDR2-beta sequence comprising the amino acid sequence of SEQ ID NO: 77, and a CDR3-beta sequence comprising the amino acid sequence of SEQ ID NO: 78. In some embodiments, the beta chain comprises a beta variable region comprising an amino acid sequence having at least 90%, 95%, or 100% identity to the amino acid sequence of SEQ ID NO: 79. In some embodiments, the beta chain comprises an amino acid sequence having at least 90%, 95%, or 100% identity to the amino acid sequence of SEQ ID NO: 80. In some embodiments, the beta chain comprises an amino acid sequence having at least 90%, 95%, or 100% identity to the amino acid sequence of SEQ ID NO: 275.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 81, a CDR2-alpha sequence comprising the amino acid sequence of SEQ ID NO: 82, a CDR3-alpha sequence comprising the amino acid sequence of SEQ ID NO: 83, a CDR1-beta sequence comprising the amino acid sequence of SEQ ID NO: 86, a CDR2-beta sequence comprising the amino acid sequence of SEQ ID NO: 87, and/or a CDR3-beta sequence comprising the amino acid sequence of SEQ ID NO: 88.

In some embodiments, the TCR comprises a CDR1-alpha sequence comprising the amino acid sequence of SEQ ID NO: 81, a CDR2-alpha sequence comprising the amino acid sequence of SEQ ID NO: 82, and a CDR3-alpha sequence comprising the amino acid sequence of SEQ ID NO: 83. In some embodiments, the alpha chain comprises an alpha variable region comprising an amino acid sequence having at least 90%, 95%, or 100% identity to the amino acid sequence of SEQ ID NO: 84. In some embodiments, the alpha chain comprises an amino acid sequence having at least 90%, 95%, or 100% identity to the amino acid sequence of SEQ ID NO: 85. In some embodiments, the alpha chain comprises an amino acid sequence having at least 90%, 95%, or 100% identity to the amino acid sequence of SEQ ID NO: 276.

In some embodiments, the TCR comprises a CDR1-beta sequence comprising the amino acid sequence of SEQ ID NO: 86, a CDR2-beta sequence comprising the amino acid sequence of SEQ ID NO: 87, and a CDR3-beta sequence comprising the amino acid sequence of SEQ ID NO: 88. In some embodiments, the beta chain comprises a beta variable region comprising an amino acid sequence having at least 90%, 95%, or 100% identity to the amino acid sequence of SEQ ID NO: 89. In some embodiments, the beta chain comprises an amino acid sequence having at least 90%, 95%, or 100% identity to the amino acid sequence of SEQ ID NO: 90. In some embodiments, the beta chain comprises an amino acid sequence having at least 90%, 95%, or 100% identity to the amino acid sequence of SEQ ID NO: 277.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 91, a CDR2-alpha sequence comprising the amino acid sequence of SEQ ID NO: 92, a CDR3-alpha sequence comprising the amino acid sequence of SEQ ID NO: 93, a CDR1-beta sequence comprising the amino acid sequence of SEQ ID NO: 96, a CDR2-beta sequence comprising the amino acid sequence of SEQ ID NO: 97, and/or a CDR3-beta sequence comprising the amino acid sequence of SEQ ID NO: 98.

In some embodiments, the TCR comprises a CDR1-alpha sequence comprising the amino acid sequence of SEQ ID NO: 91, a CDR2-alpha sequence comprising the amino acid sequence of SEQ ID NO: 92, and a CDR3-alpha sequence comprising the amino acid sequence of SEQ ID NO: 93. In some embodiments, the alpha chain comprises an alpha variable region comprising an amino acid sequence having at least 90%, 95%, or 100% identity to the amino acid sequence of SEQ ID NO: 94. In some embodiments, the alpha chain comprises an amino acid sequence having at least 90%, 95%, or 100% identity to the amino acid sequence of SEQ ID NO: 95. In some embodiments, the alpha chain comprises an amino acid sequence having at least 90%, 95%, or 100% identity to the amino acid sequence of SEQ ID NO: 278.

In some embodiments, the TCR comprises a CDR1-beta sequence comprising the amino acid sequence of SEQ ID NO: 96, a CDR2-beta sequence comprising the amino acid sequence of SEQ ID NO: 97, and a CDR3-beta sequence comprising the amino acid sequence of SEQ ID NO: 98. In some embodiments, the beta chain comprises a beta variable region comprising an amino acid sequence having at least 90%, 95%, or 100% identity to the amino acid sequence of SEQ ID NO: 99. In some embodiments, the beta chain comprises an amino acid sequence having at least 90%, 95%, or 100% identity to the amino acid sequence of SEQ ID NO: 100. In some embodiments, the beta chain comprises an amino acid sequence having at least 90%, 95%, or 100% identity to the amino acid sequence of SEQ ID NO: 279.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 110, a CDR2-alpha sequence comprising the amino acid sequence of SEQ ID NO: 111, a CDR3-alpha sequence comprising the amino acid sequence of SEQ ID NO: 112, a CDR1-beta sequence comprising the amino acid sequence of SEQ ID NO: 115, a CDR2-beta sequence comprising the amino acid sequence of SEQ ID NO: 116, and/or a CDR3-beta sequence comprising the amino acid sequence of SEQ ID NO: 117.

In some embodiments, the TCR comprises a CDR1-alpha sequence comprising the amino acid sequence of SEQ ID NO: 110, a CDR2-alpha sequence comprising the amino acid sequence of SEQ ID NO: 111, and a CDR3-alpha sequence comprising the amino acid sequence of SEQ ID NO: 112. In some embodiments, the alpha chain comprises an alpha variable region comprising an amino acid sequence having at least 90%, 95%, or 100% identity to the amino acid sequence of SEQ ID NO: 113. In some embodiments, the alpha chain comprises an amino acid sequence having at least 90%, 95%, or 100% identity to the amino acid sequence of SEQ ID NO: 114. In some embodiments, the alpha chain comprises an amino acid sequence having at least 90%, 95%, or 100% identity to the amino acid sequence of SEQ ID NO: 280.

In some embodiments, the TCR comprises a CDR1-beta sequence comprising the amino acid sequence of SEQ ID NO: 115, a CDR2-beta sequence comprising the amino acid sequence of SEQ ID NO: 116, and a CDR3-beta sequence comprising the amino acid sequence of SEQ ID NO: 117. In some embodiments, the beta chain comprises a beta variable region comprising an amino acid sequence having at least 90%, 95%, or 100% identity to the amino acid sequence of SEQ ID NO: 118. In some embodiments, the beta chain comprises an amino acid sequence having at least 90%, 95%, or 100% identity to the amino acid sequence of SEQ ID NO: 119. In some embodiments, the beta chain comprises an amino acid sequence having at least 90%, 95%, or 100% identity to the amino acid sequence of SEQ ID NO: 281.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 120, a CDR2-alpha sequence comprising the amino acid sequence of SEQ ID NO: 121, a CDR3-alpha sequence comprising the amino acid sequence of SEQ ID NO: 122, a CDR1-beta sequence comprising the amino acid sequence of SEQ ID NO: 125, a CDR2-beta sequence comprising the amino acid sequence of SEQ ID NO: 126, and/or a CDR3-beta sequence comprising the amino acid sequence of SEQ ID NO: 127.

In some embodiments, the TCR comprises a CDR1-alpha sequence comprising the amino acid sequence of SEQ ID NO: 120, a CDR2-alpha sequence comprising the amino acid sequence of SEQ ID NO: 121, and a CDR3-alpha sequence comprising the amino acid sequence of SEQ ID NO: 122. In some embodiments, the alpha chain comprises an alpha variable region comprising an amino acid sequence having at least 90%, 95%, or 100% identity to the amino acid sequence of SEQ ID NO: 123. In some embodiments, the alpha chain comprises an amino acid sequence having at least 90%, 95%, or 100% identity to the amino acid sequence of SEQ ID NO: 124. In some embodiments, the alpha chain comprises an amino acid sequence having at least 90%, 95%, or 100% identity to the amino acid sequence of SEQ ID NO: 282.

In some embodiments, the TCR comprises a CDR1-beta sequence comprising the amino acid sequence of SEQ ID NO: 125, a CDR2-beta sequence comprising the amino acid sequence of SEQ ID NO: 126, and a CDR3-beta sequence comprising the amino acid sequence of SEQ ID NO: 127. In some embodiments, the beta chain comprises a beta variable region comprising an amino acid sequence having at least 90%, 95%, or 100% identity to the amino acid sequence of SEQ ID NO: 128. In some embodiments, the beta chain comprises an amino acid sequence having at least 90%, 95%, or 100% identity to the amino acid sequence of SEQ ID NO: 129. In some embodiments, the beta chain comprises an amino acid sequence having at least 90%, 95%, or 100% identity to the amino acid sequence of SEQ ID NO: 283.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 130, a CDR2-alpha sequence comprising the amino acid sequence of SEQ ID NO: 131, a CDR3-alpha sequence comprising the amino acid sequence of SEQ ID NO: 132, a CDR1-beta sequence comprising the amino acid sequence of SEQ ID NO: 135, a CDR2-beta sequence comprising the amino acid sequence of SEQ ID NO: 136, and/or a CDR3-beta sequence comprising the amino acid sequence of SEQ ID NO: 137.

In some embodiments, the TCR comprises a CDR1-alpha sequence comprising the amino acid sequence of SEQ ID NO: 130, a CDR2-alpha sequence comprising the amino acid sequence of SEQ ID NO: 131, and a CDR3-alpha sequence comprising the amino acid sequence of SEQ ID NO: 132. In some embodiments, the alpha chain comprises an alpha variable region comprising an amino acid sequence having at least 90%, 95%, or 100% identity to the amino acid sequence of SEQ ID NO: 133. In some embodiments, the alpha chain comprises an amino acid sequence having at least 90%, 95%, or 100% identity to the amino acid sequence of SEQ ID NO: 134. In some embodiments, the alpha chain comprises an amino acid sequence having at least 90%, 95%, or 100% identity to the amino acid sequence of SEQ ID NO: 284.

In some embodiments, the TCR comprises a CDR1-beta sequence comprising the amino acid sequence of SEQ ID NO: 135, a CDR2-beta sequence comprising the amino acid sequence of SEQ ID NO: 136, and a CDR3-beta sequence comprising the amino acid sequence of SEQ ID NO: 137. In some embodiments, the beta chain comprises a beta variable region comprising an amino acid sequence having at least 90%, 95%, or 100% identity to the amino acid sequence of SEQ ID NO: 138. In some embodiments, the beta chain comprises an amino acid sequence having at least 90%, 95%, or 100% identity to the amino acid sequence of SEQ ID NO: 139. In some embodiments, the beta chain comprises an amino acid sequence having at least 90%, 95%, or 100% identity to the amino acid sequence of SEQ ID NO: 285.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 140, a CDR2-alpha sequence comprising the amino acid sequence of SEQ ID NO: 141, a CDR3-alpha sequence comprising the amino acid sequence of SEQ ID NO: 142, a CDR1-beta sequence comprising the amino acid sequence of SEQ ID NO: 145, a CDR2-beta sequence comprising the amino acid sequence of SEQ ID NO: 146, and/or a CDR3-beta sequence comprising the amino acid sequence of SEQ ID NO: 147.

In some embodiments, the TCR comprises a CDR1-alpha sequence comprising the amino acid sequence of SEQ ID NO: 140, a CDR2-alpha sequence comprising the amino acid sequence of SEQ ID NO: 141, and a CDR3-alpha sequence comprising the amino acid sequence of SEQ ID NO: 142. In some embodiments, the alpha chain comprises an alpha variable region comprising an amino acid sequence having at least 90%, 95%, or 100% identity to the amino acid sequence of SEQ ID NO: 143. In some embodiments, the alpha chain comprises an amino acid sequence having at least 90%, 95%, or 100% identity to the amino acid sequence of SEQ ID NO: 144. In some embodiments, the alpha chain comprises an amino acid sequence having at least 90%, 95%, or 100% identity to the amino acid sequence of SEQ ID NO: 286.

In some embodiments, the TCR comprises a CDR1-beta sequence comprising the amino acid sequence of SEQ ID NO: 145, a CDR2-beta sequence comprising the amino acid sequence of SEQ ID NO: 146, and a CDR3-beta sequence comprising the amino acid sequence of SEQ ID NO: 147. In some embodiments, the beta chain comprises a beta variable region comprising an amino acid sequence having at least 90%, 95%, or 100% identity to the amino acid sequence of SEQ ID NO: 148. In some embodiments, the beta chain comprises an amino acid sequence having at least 90%, 95%, or 100% identity to the amino acid sequence of SEQ ID NO: 149. In some embodiments, the beta chain comprises an amino acid sequence having at least 90%, 95%, or 100% identity to the amino acid sequence of SEQ ID NO: 287.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 150, a CDR2-alpha sequence comprising the amino acid sequence of SEQ ID NO: 151, a CDR3-alpha sequence comprising the amino acid sequence of SEQ ID NO: 152, a CDR1-beta sequence comprising the amino acid sequence of SEQ ID NO: 155, a CDR2-beta sequence comprising the amino acid sequence of SEQ ID NO: 156, and/or a CDR3-beta sequence comprising the amino acid sequence of SEQ ID NO: 157.

In some embodiments, the TCR comprises aa CDR1-alpha sequence comprising the amino acid sequence of SEQ ID NO: 150, a CDR2-alpha sequence comprising the amino acid sequence of SEQ ID NO: 151, and a CDR3-alpha sequence comprising the amino acid sequence of SEQ ID NO: 152. In some embodiments, the alpha chain comprises an alpha variable region comprising an amino acid sequence having at least 90%, 95%, or 100% identity to the amino acid sequence of SEQ ID NO: 153. In some embodiments, the alpha chain comprises an amino acid sequence having at least 90%, 95%, or 100% identity to the amino acid sequence of SEQ ID NO: 154. In some embodiments, the alpha chain comprises an amino acid sequence having at least 90%, 95%, or 100% identity to the amino acid sequence of SEQ ID NO: 288.

In some embodiments, the TCR comprises a CDR1-beta sequence comprising the amino acid sequence of SEQ ID NO: 155, a CDR2-beta sequence comprising the amino acid sequence of SEQ ID NO: 156, and a CDR3-beta sequence comprising the amino acid sequence of SEQ ID NO: 157. In some embodiments, the beta chain comprises a beta variable region comprising an amino acid sequence having at least 90%, 95%, or 100% identity to the amino acid sequence of SEQ ID NO: 158. In some embodiments, the beta chain comprises an amino acid sequence having at least 90%, 95%, or 100% identity to the amino acid sequence of SEQ ID NO: 159. In some embodiments, the beta chain comprises an amino acid sequence having at least 90%, 95%, or 100% identity to the amino acid sequence of SEQ ID NO: 289.

Some aspects relate to an engineered regulatory T cell that comprises an exogenous TCR that binds to a GAD65 peptide complexed with an MHC molecule, wherein the TCR comprises an alpha chain and a beta chain, and wherein the TCR comprises: a CDR1-alpha sequence comprising the amino acid sequence of SEQ ID NO: 160, a CDR2-alpha sequence comprising the amino acid sequence of SEQ ID NO: 161, a CDR3-alpha sequence comprising the amino acid sequence of SEQ ID NO: 162, a CDR1-beta sequence comprising the amino acid sequence of SEQ ID NO: 165, a CDR2-beta sequence comprising the amino acid sequence of SEQ ID NO: 166, and/or a CDR3-beta sequence comprising the amino acid sequence of SEQ ID NO: 167.

In some embodiments, the TCR comprises a CDR1-alpha sequence comprising the amino acid sequence of SEQ ID NO: 160, a CDR2-alpha sequence comprising the amino acid sequence of SEQ ID NO: 161, and a CDR3-alpha sequence comprising the amino acid sequence of SEQ ID NO: 162. In some embodiments, the alpha chain comprises an alpha variable region comprising an amino acid sequence having at least 90%, 95%, or 100% identity to the amino acid sequence of SEQ ID NO: 163. In some embodiments, the alpha chain comprises an amino acid sequence having at least 90%, 95%, or 100% identity to the amino acid sequence of SEQ ID NO: 164. In some embodiments, the alpha chain comprises an amino acid sequence having at least 90%, 95%, or 100% identity to the amino acid sequence of SEQ ID NO: 290.

In some embodiments, the TCR comprises a CDR1-beta sequence comprising the amino acid sequence of SEQ ID NO: 165, a CDR2-beta sequence comprising the amino acid sequence of SEQ ID NO: 166, and a CDR3-beta sequence comprising the amino acid sequence of SEQ ID NO: 167. In some embodiments, the beta chain comprises a beta variable region comprising an amino acid sequence having at least 90%, 95%, or 100% identity to the amino acid sequence of SEQ ID NO: 168. In some embodiments, the beta chain comprises an amino acid sequence having at least 90%, 95%, or 100% identity to the amino acid sequence of SEQ ID NO: 169. In some embodiments, the beta chain comprises an amino acid sequence having at least 90%, 95%, or 100% identity to the amino acid sequence of SEQ ID NO: 291.

### Cell Populations

In some embodiments, the disclosure provides cell populations (e.g., isolated cell populations) comprising regulatory T cells, including stable regulatory T cells, transfected with a nucleic acid expressing an exogenous human TCR that binds specifically to a GAD65 peptide (e.g., GAD65 555-567) complexed with an MHC (e.g., a TCR as described in Table 1). In some embodiments, the isolated cell populations comprising stable regulatory T cells comprise a hypomethylated TSDR at an endogenous FOXP3 locus. In some embodiments, stable regulatory T cells further comprise an exogenous human TCR that binds specifically to a target peptide complexed with an MHC molecule.

An isolated cell population is a cell population that is removed from a human body. Thus, it is considered “isolated” from the human body. A population of cells may be isolated (e.g., obtained from) a subject, or from a biological sample obtained from the subject, for example, using any known cell collection method, such as apheresis. An isolated cell population of the disclosure may be subjected to the methods described herein to produce an isolated cell population a higher number of regulatory T cells (e.g., stable regulatory T cells) relative to a population of cells obtained directly from a subject, or from a biological sample obtained from the subject, by apheresis, for example.

In some embodiments, an isolated cell population comprises CD25^{+/high}CD4⁺CD127^{-/lo} regulatory T cells. In some embodiments, an isolated cell population comprises CD25^{+/high}CD4⁺CD127^{-/lo}/FOXP3⁺ regulatory T cells. In some embodiments, an isolated cell population comprises CD25^{+/high}CD4⁺CD127^{-/lo}/FOXP3⁺/CD45RA⁺ regulatory T cells.

In some embodiments, at least 50%, at least 60%, at least 70%, at least 75%, at least 80%, at least 85%, at least 90%, at least 95%, at least 96%, at least 97%, at least 98%, or at least 99% of the cells of an isolated cell population are stable regulatory T cells comprising a hypomethylated TSDR at an endogenous FOXP3 locus.

In some embodiments, at least 50%, at least 60%, at least 70%, at least 75%, at least 80%, at least 85%, at least 90%, at least 95%, at least 96%, at least 97%, at least 98%, or at least 99% of the cells of an isolated cell population are CD25^{+/high}CD4⁺CD127^{-/lo} regulatory T cells. In some embodiments, at least 50%, at least 60%, at least 70%, at least 75%, at least 80%, at least 85%, at least 90%, at least 95%, at least 96%, at least 97%, at least 98%, or at least 99% of the cells of an isolated cell population are CD25^{+/high}CD4⁺CD127^{-/lo}/FOXP3⁺ regulatory T cells.

In some embodiments, at least 10%, at least 25%, at least 50%, at least 75%, at least 80%, at least 85%, at least 90%, at least 95%, at least 96%, at least 97%, at least 98%, or at least 99% of the stable regulatory T cells of a population are CD45RA⁺. In some embodiments, at least 10%, at least 25%, at least 50%, at least 75%, at least 80%, at least 85%, at least 90%, at least 95%, at least 96%, at least 97%, at least 98%, or at least 99% of the stable regulatory T cells of a population are CD25^{+/high}CD4⁺CD127^{-/lo}/CD45RA⁺ regulatory T cells. In some embodiments, at least 10%, at least 25%, at least 50%, at least 75%, at least 80%, at least 85%, at least 90%, at least 95%, at least 96%, at least 97%, at least 98%, or at least 99% of the stable regulatory T cells of a population are CD25^{+/high}CD4⁺CD127^{-/lo}/FOXP3+/CD45RA⁺.

Stable regulatory T cells of a cell population, in some embodiments, are non-engineered at the endogenous FOXP3 locus. A FOXP3 locus is considered engineered if it has been edited (e.g., directly edited) using a gene editing technology such as, for example, homology-directed repair (HDR)-based gene editing, or if a cell has been otherwise genetically modified to increase or stabilize expression from the FOXP3 locus, relative to endogenous (naturally occurring) FOXP3 expression. In some embodiments, endogenous FOXP3 in the regulatory T cells of the population has not been gene edited. Thus, in some embodiments, fewer than (less than) 10% of the cells of the isolated cell population express FOXP3 protein from an engineered FOXP3 locus. In some embodiments, fewer than (less than) 9%, 8%, 7%, 6%, 5%, 4%, 3%, 2%, or 1% of the cells of the isolated cell population express FOXP3 protein from an engineered FOXP3 locus. In some embodiments, fewer than (less than) 0.5% or fewer than (less than) 0.1% of the cells of the isolated cell population express FOXP3 protein from an engineered FOXP3 locus.

In some embodiments, an isolated cell population comprises at least 1×10², at least 1×10³, at least 1×10⁴, at least 1×10⁵, at least 1×10⁶, at least 1×10⁷, at least 1×10⁸, at least 1×10⁹, or at least 1×10¹⁰ stable regulatory T cells. In some embodiments, an isolated cell population comprises 1×10² to 1×10¹⁰, 1×10³ to 1×10¹⁰, 1×10⁴ to 1×10¹⁰, 1×10⁵ to 1×10¹⁰, 1×10⁶ to 1×10¹⁰, 1×10⁷ to 1×10¹⁰, 1×10⁸ to 1×10¹⁰, 1×10⁵ to 1×10⁹, 1×10⁶ to 1×10⁸, 1×10⁷ to 1×10¹⁰, or 1×10⁴ to 1×10⁶ stable regulatory T cells. In some embodiments, an isolated cell population comprises 1×10⁶ to 1×10¹⁰ stable regulatory T cells.

In some embodiments, a subset of an isolated cell population are stable regulatory T cells (e.g., having a hypomethylated TSDR) at an endogenous FOXP3 and expressing CD4, CD25, and FOXP3). In some embodiments, the stable regulatory T cells within an isolated cell population maintain a hypomethylated TSDR at an endogenous FOXP3 locus over time, as described elsewhere herein.

In some embodiments, the stable regulatory T cells within an isolated cell population retain markers of stability in the presence of pro-inflammatory conditions (e.g., in presence of one or more pro-inflammatory cytokines). In some embodiments, stable regulatory T cells within an isolated cell population maintain a hypomethylated TSDR at an endogenous FOXP3 locus in the presence of pro-inflammatory conditions (e.g., in presence of one or more pro-inflammatory cytokines). In some embodiments, stable regulatory T cells within an isolated cell population maintain a hypomethylated TSDR at an endogenous FOXP3 locus in the presence of pro-inflammatory conditions (e.g., in presence of one or more pro-inflammatory cytokines). In some embodiments, the stable regulatory T cells within an isolated cell population maintain a hypomethylated TSDR at an endogenous FOXP3 locus and express CD4, CD25, and FOXP3 in the presence of pro-inflammatory conditions (e.g., in presence of one or more pro-inflammatory cytokines). In some embodiments, the stable regulatory T cells within an isolated cell population maintain a hypomethylated TSDR at an endogenous FOXP3 locus in the presence of pro-inflammatory conditions (e.g., in presence of one or more pro-inflammatory cytokines) for at least 3, 4, 5, 6, 7, 8, 9, 10, 11, 12, 13, 14, 15, 20, 21, 22, 23, 24, or 25 days.

At least 50%, at least 60%, at least 70%, at least 80%, at least 90%, at least 95%, or at least 98% of the cells of an isolated cell population may be stable regulatory T cells. Thus, in some embodiments, at least 50%, at least 60%, at least 70%, at least 80%, at least 90%, at least 95%, or at least 98% of the cells of an isolated cell population comprises a hypomethylated TSDR at a FOXP3 locus. In some embodiments, at least 50%, at least 60%, at least 70%, at least 80%, at least 90%, at least 95%, or at least 98% of the cells of an isolated cell population comprises a hypomethylated TSDR at a FOXP3 locus for at least 1, 2, 3, 4, 5, 6, 7, 8, 9, 10, 11, 12, 13, 14, 15, 16, 17, 18, 19, or 20 days (e.g., after being obtained from a subject or following transduction with a nucleic acid expressing a TCR).

In some embodiments, at least 50% of the cells of an isolated cell population comprises a hypomethylated TSDR at a FOXP3 locus for at least 5 days. In some embodiments, at least 50% of the cells of an isolated cell population comprises a hypomethylated TSDR at a FOXP3 locus for at least 10 days. In some embodiments, at least 50% of the cells of an isolated cell population comprises a hypomethylated TSDR at a FOXP3 locus for at least 15 days. In some embodiments, at least 60% of the cells of an isolated cell population comprises a hypomethylated TSDR at a FOXP3 locus for at least 5 days. In some embodiments, at least 60% of the cells of an isolated cell population comprises a hypomethylated TSDR at a FOXP3 locus for at least 10 days. In some embodiments, at least 60% of the cells of an isolated cell population comprises a hypomethylated TSDR at a FOXP3 locus for at least 15 days. In some embodiments, at least 70% of the cells of an isolated cell population comprises a hypomethylated TSDR at a FOXP3 locus for at least 5 days. In some embodiments, at least 70% of the cells of an isolated cell population comprises a hypomethylated TSDR at a FOXP3 locus for at least 10 days. In some embodiments, at least 70% of the cells of an isolated cell population comprises a hypomethylated TSDR at a FOXP3 locus for at least 15 days. In some embodiments, at least 80% of the cells of an isolated cell population comprises a hypomethylated TSDR at a FOXP3 locus for at least 5 days. In some embodiments, at least 80% of the cells of an isolated cell population comprises a hypomethylated TSDR at a FOXP3 locus for at least 10 days. In some embodiments, at least 80% of the cells of an isolated cell population comprises a hypomethylated TSDR at a FOXP3 locus for at least 15 days. In some embodiments, at least 90% of the cells of an isolated cell population comprises a hypomethylated TSDR at a FOXP3 locus for at least 5 days. In some embodiments, at least 90% of the cells of an isolated cell population comprises a hypomethylated TSDR at a FOXP3 locus for at least 10 days. In some embodiments, at least 90% of the cells of an isolated cell population comprises a hypomethylated TSDR at a FOXP3 locus for at least 15 days.

In some embodiments, the percentage of cells of the isolated cell population comprising the stable regulatory T cells does not decrease by more than 10, 9, 8, 7, 6, 5, 4, 3, 2, or 1 percentage point(s) during at least 5, 6, 7, 8, 9, 10, or 11 days of expansion following transduction of the stable regulatory T cells with a nucleic acid expressing an exogenous human TCR. In some embodiments, the percentage of cells of the isolated cell population comprising the stable regulatory T cells increases by 1, 2, 3, 4, or 5 percentage point(s) during at least 5, 6, 7, 8, 9, 10, or 11 days of expansion following transduction of the stable regulatory T cells with a nucleic acid expressing an exogenous human TCR. In some embodiments, the percentage of cells of the isolated cell population comprising the stable regulatory T cells does not decrease by more than 10 percentage points during at least 5 days of expansion following transduction of the stable regulatory T cells with a nucleic acid expressing an exogenous human TCR. In some embodiments, the percentage of cells of the isolated cell population comprising the stable regulatory T cells does not decrease by more than 10 percentage points during at least 10 days of expansion following transduction of the stable regulatory T cells with a nucleic acid expressing an exogenous human TCR. In some embodiments, the percentage of cells of the isolated cell population comprising the stable regulatory T cells does not decrease by more than 10 percentage points during at least 15 days of expansion following transduction of the stable regulatory T cells with a nucleic acid expressing an exogenous human TCR. In some embodiments, the percentage of cells of the isolated cell population comprising the stable regulatory T cells does not decrease by more than 5 percentage points during at least 5 days of expansion following transduction of the stable regulatory T cells with a nucleic acid expressing an exogenous human TCR. In some embodiments, the percentage of cells of the isolated cell population comprising the stable regulatory T cells does not decrease by more than 5 percentage points during at least 10 days of expansion following transduction of the stable regulatory T cells with a nucleic acid expressing an exogenous human TCR. In some embodiments, the percentage of cells of the isolated cell population comprising the stable regulatory T cells does not decrease by more than 5 percentage points during at least 15 days of expansion following transduction of the stable regulatory T cells with a nucleic acid expressing an exogenous human TCR. In some embodiments, the percentage of cells of the isolated cell population comprising the stable regulatory T cells does not decrease by more than 1 percentage point during at least 5 days of expansion following transduction of the stable regulatory T cells with a nucleic acid expressing an exogenous human TCR. In some embodiments, the percentage of cells of the isolated cell population comprising the stable regulatory T cells does not decrease by more than 1 percentage point during at least 10 days of expansion following transduction of the stable regulatory T cells with a nucleic acid expressing an exogenous human TCR. In some embodiments, the percentage of cells of the isolated cell population comprising the stable regulatory T cells does not decrease by more than 1 percentage point during at least 15 days of expansion following transduction of the stable regulatory T cells with a nucleic acid expressing an exogenous human TCR.

The percentage of stable regulatory T cells relative to total cells in an isolated cell population comprising regulatory T cells may be assessed about 1, about 6, about 12, about 24, about 36, about 48, about 72, about 96, or about 120 hours after transduction of cells with a nucleic acid expressing an exogenous human TCR. The percentage of stable regulatory T cells relative to total cells in an isolated cell population comprising regulatory T cells may be assessed 4, 5, 6, 7, 8, 9, 10, 11, 12, 13, or 14 days after transduction of cells with a nucleic acid expressing an exogenous human TCR. The percentage of stable regulatory T cells relative to total cells in an isolated cell population comprising regulatory T cells may be assessed about 1-21, 1-7, 4-14, 4-7, 7-10, 7-14, 10-21, or 14-21 days after transduction of cells with a nucleic acid expressing an exogenous human TCR.

In some embodiments, at least 10%, at least 25%, at least 50%, at least 60%, at least 70%, at least 80%, at least 90%, or at least 100% of the cells of an isolated cell population comprise an unmodified endogenous FOXP3 gene locus. In some embodiments, less than 25%, less than 20%, less than 15%, less than 10%, less than 5%, less than 2%, or less than 1% of the cells of the isolated cell population comprise a modified endogenous FOXP3 gene locus. An unmodified endogenous FOXP3 gene locus is a naturally occurring FOXP3 gene locus, which has not been edited, for example, using a gene editing or transgenic technique.

In some embodiments, at least 20%, at least 30%, at least 40%, at least 50%, at least 60%, at least 70%, at least 80%, at least 90%, or at least 100% of the cells of the isolated cell population express physiological levels of functional FOXP3 protein. A physiological level of functional FOXP3 protein is the protein level expressed from an unmodified (naturally occurring) FOXP3 gene locus.

In some embodiments, at least 20%, at least 30%, at least 40%, at least 50%, at least 60%, at least 70%, at least 80%, at least 90%, or at least 100% of the cells of the isolated cell population do not comprise an exogenous, e.g., ectopic, FOXP3 gene or an exogenous, e.g., ectopic, FOXP3 protein. In some embodiments, at least 20%, at least 30%, at least 40%, at least 50%, at least 60%, at least 70%, at least 80%, at least 90%, or at least 100% of the cells of the isolated cell population do not comprise ectopic expression of FOXP3. Ectopic expression of FOXP3 refers to expression that does not naturally occur in a cell. Thus, ectopic expression results, for example, from expression of an exogenous transgene or expression from an endogenous FOXP3 gene that has been modified at the FOXP3 gene locus, e.g., modified at the promoter region of the FOXP3 gene.

In some embodiments, less than 40%, less than 30%, less than 20%, less than 10%, less than 5%, less than 2%, or less than 1% of the cells of the isolated cell population comprise an exogenous, e.g., ectopic, FOXP3 gene or an exogenous, e.g., ectopic, FOXP3 protein. An exogenous gene or protein is one that has been introduced to a cell, for example, by transfection, transduction, or electroporation.

In some embodiments, at least 5%, at least 10%, at least 15%, at least 20%, at least 30%, at least 40%, at least 50%, at least 60%, at least 70%, at least 80%, at least 90%, or at least 95% of the regulatory T cells of an isolated cell population of regulatory T cells express an exogenous TCR (e.g., following transduction of the isolated cell population with a nucleic acid expressing an exogenous TCR). In some embodiments, 10%-60% or 20%-50% of the regulatory T cells of an isolated cell population of regulatory T cells express an exogenous TCR. In some embodiments, at least 5%, at least 10%, at least 15%, at least 20%, at least 30%, at least 40%, at least 50%, at least 60%, at least 70%, at least 80%, at least 90%, or at least 95% of the stable regulatory T cells of an isolated cell population of regulatory T cells express an exogenous TCR (e.g., following transduction of the isolated cell population with a nucleic acid expressing an exogenous TCR). In some embodiments, 10%-60% or 20%-50% of the stable regulatory T cells of an isolated cell population of regulatory T cells express an exogenous TCR.

In some embodiments, the TSDR at the endogenous FOXP3 locus of stable regulatory T cells of an isolated cell population of regulatory T cells remains hypomethylated until administration of the isolated cell population to a subject. In some embodiments, the TSDR at the endogenous FOXP3 locus of stable regulatory T cells of an isolated cell population of regulatory T cells remains hypomethylated following a cryopreservation freeze-thaw cycle.

In some embodiments, regulatory T cells (e.g., stable regulatory T cells) of an isolated cell population exhibit one or more cellular functions that are associated with regulatory T cells when activated by binding the pMHC. Non-limiting examples of such cellular functions include cytokine secretion activity, expression of certain activation markers, and suppression activity. Cytokine secretion activity simply refers to the secretion of certain anti-inflammatory cytokines, such as IL-10, IL-4, IL-6, IL-11, and IL-13. Activation markers include, but are not limited to CD69, 4-1BB, CD25, CD71, or CTLA-4. A regulatory T cell expresses one or more of these markers when it comes into contact with a target peptide complexed with an MHC molecule, for example.

Suppression activity refers to the suppression of activation, proliferation and cytokine production of non-regulatory T cells (e.g., CD8⁺ T cells and CD4⁺ conventional T cells) in part to suppress the immune system from becoming overactive. Regulatory T cells exhibit suppression activity when contacted with a target peptide complexed with MHC that binds to an exogenous TCR expressed by the regulatory T cells. In some embodiments, the target peptide is presented by a cell expressing the MHC. For example, in some embodiments, regulatory T cells suppress the activation, proliferation and cytokine production of conventional T cells having specificity towards a shared target peptide complexed with an MHC molecule, e.g., a shared target peptide presented by an Antigen Presenting Cell (APC). In some embodiments, regulatory T cells suppress proliferation and growth of non-regulatory T cells by at least 25%, at least 40%, at least 50%, at least 60%, at least 70%, or at least 80%, relative to a control (e.g., non-regulatory T cells in the absence of regulatory T cells).

In some embodiments, regulatory T cells suppress the production of interferon (IFN)-gamma from conventional T cells by at least 25%, at least 40%, at least 50%, at least 60%, at least 70%, or at least 80%, relative to a control (e.g., conventional T cells in the absence of regulatory T cells). In some embodiments, regulatory T cells suppress the production of IFN-gamma from conventional T cells by 50%-99%, 75%-99%, or 80%-100% relative to a control (e.g., when present in a population comprising a ratio of 1:1 to 1:8 regulatory T cells compared to conventional T cells). In some embodiments, regulatory T cells suppress the production of CD71 from conventional T cells by at least 20%, at least 30%, at least 40%, at least 50%, at least 60%, or at least 70%, relative to a control (e.g., conventional T cells in the absence of regulatory T cells). In some embodiments, regulatory T cells suppress the production of CD71 of conventional T cells by 20%-90% or 30%-80% relative to a control (e.g., when present in a population comprising a ratio of 1:1 to 1:8 regulatory T cells compared to conventional T cells). In some embodiments, regulatory T cells suppress the production of CD25 from conventional T cells by at least 20%, at least 30%, at least 40%, at least 50%, at least 60%, or at least 70%, relative to a control (e.g., conventional T cells in the absence of regulatory T cells). In some embodiments, regulatory T cells suppress the production of CD25 of conventional T cells by 40%-70% relative to a control (e.g., when present in a population comprising a ratio of 1:1 to 1:8 regulatory T cells compared to conventional T cells).

In some embodiments, regulatory T cells exhibit cytokine secretion activity (e.g., secretion of IL-10) when contacted with a target peptide complexed with an MHC molecule, e.g., a target peptide presented by an APC, that binds to an exogenous TCR expressed by the regulatory T cells. In some embodiments, regulatory T cells exhibit expression of activation markers when contacted with a target peptide complexed with MHC that binds to an exogenous TCR expressed by the regulatory T cells. For example, in some embodiments, regulatory T cells exhibit expression of CD69, 4-1BB, CD25, CD71, and/or CTLA-4 when contacted with a target peptide complexed with MHC that binds to an exogenous TCR expressed by the regulatory T cells. In some embodiments, regulatory T cells exhibit suppression activity when contacted with a target peptide complexed with MHC that binds to an exogenous TCR expressed by the regulatory T cells. For example, in some embodiments, regulatory T cells suppress the activation of conventional T cells having specificity towards a shared target peptide complexed with MHC.

The cellular functions of stable regulatory T cells may be assessed about 1, about 6, about 12, about 24, about 36, about 48, about 72, about 96, or about 120 hours after transduction of cells with a nucleic acid expressing an exogenous human TCR. The cellular functions of stable regulatory T cells may be assessed 4, 5, 6, 7, 8, 9, 10, 11, 12, 13, or 14 days after transduction of cells with a nucleic acid expressing an exogenous human TCR. The cellular functions of stable regulatory T cells may be assessed about 1-21, 1-7, 4-14, 4-7, 7-10, 7-14, 10-21, or 14-21 days after transduction of cells with a nucleic acid expressing an exogenous human TCR.

In some embodiments, transduced regulatory T cells (e.g., transduced regulatory T cells of an isolated population) retain their cellular functionality (e.g., ability to be activated) following a cryopreservation freeze-thaw cycle. In some embodiments, transduced regulatory T cells can be activated and/or expanded following a cryopreservation freeze-thaw cycle. In some embodiments, regulatory T cells can exhibit cytokine secretion activity, expression of certain activation markers, and/or suppression activity following a cryopreservation freeze-thaw cycle.

In some embodiments, at least 5%, at least 10%, at least 25%, at least 50%, at least 75%, at least 80%, at least 85%, at least 90%, at least 95%, at least 96%, at least 97%, at least 98%, or at least 99% of a population of regulatory T cells are CD25^{+/high}CD4⁺CD127^{-/lo} prior to activation and/or transduction with a nucleic acid expressing an exogenous human T cell receptor. In some embodiments, at least 5%, at least 10%, at least 25%, at least 50%, at least 75%, at least 80%, at least 85%, at least 90%, at least 95%, at least 96%, at least 97%, at least 98%, or at least 99% of a population of regulatory T cells are CD25^{+/high}CD4⁺CD127^{-/lo}CD45RA⁺ prior to activation and/or transduction with a nucleic acid expressing an exogenous human T cell receptor.

An isolated cell population comprising regulatory T cells may comprise a minority amount of non-regulatory T cells (e.g., conventional T cells). Non-regulatory T cells may be NK T cells, B cells, CD8⁺ T cells, neutrophils, eosinophils, CD14⁺ cells, or conventional (CD4⁺) T cells that are derived from peripheral blood and lymph nodes. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are non-regulatory T cells. In some embodiments, about 0.01% to about 0.1%, about 0.1% to about 0.5%, about 0.5% to about 1%, about 0.5% to about 10%, about 2% to about 5%, or about 5% to about 10% of the cells of an isolated cell population comprising regulatory T cells are non-regulatory T cells. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are non-regulatory T cells that comprise an exogenous human TCR (e.g., an engineered human TCR of the disclosure). In some embodiments, 10% or fewer of the cells of an isolated cell population comprising regulatory T cells are non-regulatory T cells that comprise an exogenous human TCR.

A conventional T cell commonly produces IL-2 and other interleukin factors. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are conventional T cells. In some embodiments, about 0.01% to about 0.1%, about 0.1% to about 0.5%, about 0.5% to about 1%, about 0.5% to about 10%, about 2% to about 5%, or about 5% to about 10% of the cells of an isolated cell population comprising regulatory T cells are conventional T cells. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are conventional T cells that comprise an exogenous human TCR. In some embodiments, an isolated cell population comprising regulatory T cells comprises an undetectable amount of conventional T cells. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are conventional CD4+ T cells. In some embodiments, 20% or fewer of the cells of an isolated cell population comprising regulatory T cells are conventional CD4+ T cells. In some embodiments, an isolated cell population comprising regulatory T cells comprises an undetectable amount of conventional CD4⁺ T cells. In some embodiments, an isolated cell population comprising regulatory T cells comprises an undetectable amount of B cells. In some embodiments, an isolated cell population comprising regulatory T cells comprises an undetectable amount of NK T cells. In some embodiments, an isolated cell population comprising regulatory T cells comprises an undetectable amount of CD14⁺ cells. In some embodiments, an isolated cell population comprising regulatory T cells comprises an undetectable number of eosinophils. In some embodiments, an isolated cell population comprising regulatory T cells comprises an undetectable number of neutrophils.

In some embodiments, fewer than 10%, fewer than 5%, fewer than 2%, fewer than 1%, fewer than 0.5%, fewer than 0.1%, or fewer than 0.01% of the cells of an isolated cell population comprising regulatory T cells are CD8+ T cells. In some embodiments, 5% or fewer of the cells of an isolated cell population are CD8+ T cells. In some embodiments, an isolated population of cells comprising regulatory T cells comprises an undetectable amount of CD8+ T cells. A cell type is considered undetectable if the presence of the cell in a sample cannot by detected by flow cytometry, for example, based on one or more cell-specific biomarker(s).

The percentage of conventional T cells or other non-regulatory T cells relative to total cells (or relative to stable regulatory T cells) may be assessed about 1, about 6, about 12, about 24, about 36, about 48, about 72, about 96, or about 120 hours after transduction of cells with a nucleic acid expressing an exogenous human TCR. The percentage of conventional T cells or other non-regulatory T cells relative to total cells (or relative to stable regulatory T cells) may be assessed 4, 5, 6, 7, 8, 9, 10, 11, 12, 13, or 14 days after transduction of cells with a nucleic acid expressing an exogenous human TCR. The percentage of conventional T cells or other non-regulatory T cells relative to total cells (or relative to stable regulatory T cells) may be assessed about 1-21, 1-7, 4-14, 4-7, 7-10, 7-14, 10-21, or 14-21 days after transduction of cells with a nucleic acid expressing an exogenous human TCR.

In some embodiments, the ratio of regulatory T cells to conventional T cells in an isolate population of cells comprising regulatory T cells is at least 5:1, at least 10:1, at least 15:1, at least 20:1, at least 25:1, at least 30:1, at least 35:1, at least 40:1, at least 45:1, at least 50:1, at least 60:1, at least 70:1, at least 80:1, at least 90:1, or at least 100:1.

### Pharmaceutical Compositions

In some embodiments, the disclosure provides pharmaceutical compositions comprising any one of the TCRs described herein (e.g., a TCR as described in Table 1). In some embodiments, the disclosure provides pharmaceutical compositions comprising the isolated cell populations of regulatory T cells (e.g., stable regulatory T cells) described herein. In some embodiments, a pharmaceutical composition comprises a cell population of regulatory T cells (e.g., stable regulatory T cells) described herein and a pharmaceutically acceptable excipient.

As used herein, a pharmaceutically acceptable excipient may also be referred to as a pharmaceutically acceptable carrier, pharmaceutically acceptable diluent, or pharmaceutically acceptable adjuvant. Formulation of pharmaceutically acceptable excipients and carrier solutions is well-known to those of skill in the art, as is the development of suitable dosing and treatment regimens for using the compositions described herein in a variety of treatment regimens.

The pharmaceutical compositions typically should be sterile and stable under the conditions of manufacture and storage. Sterile injectable formulations may be prepared using a non-toxic parenterally acceptable diluent or solvent. A pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers which are non-toxic to the subjects at the dosages and concentrations employed. In some embodiments, the pharmaceutical composition may comprise an organic solvent, such as but not limited to, methyl acetate, dimethyl sulfoxide (DMSO), N,N-dimethylformamide (DMF), dimethoxyethane (DME), and dimethylacetamide, including mixtures or combinations thereof.

A pharmaceutical composition may comprise an effective amount of stable regulatory T cells that is sufficient to elicit a desired biological response. For example, in some embodiments, an effective amount of stable regulatory T cells as described herein may refer to a number of cells that is sufficient to improve a symptom associated with Type 1 Diabetes. As will be appreciated by the skilled artisan, an effective amount of a solution or preparation provided herein may vary depending on various factors as, for example, on the desired biological response, e.g., on the specific disease being treated, the specific symptom to be alleviated, on the cell or tissue being targeted, and on the subject's age, gender, and general health status.

In some embodiments, an effective amount of stable regulatory T cells (e.g., stable regulatory T cells that (a) comprise an exogenous human T cell receptor (TCR) that binds specifically to a target peptide complexed with an MHC molecule and (b) comprise a hypomethylated TSDR at an endogenous FOXP3 locus) comprises at least 1×10², at least 1×10³, at least 1×10⁴, at least 1×10⁵, at least 1×10⁶, at least 1×10⁷, at least 1×10⁸, at least 1×10⁹, or at least 1×10¹⁰ stable regulatory T cells. In some embodiments, an effective amount of stable regulatory T cells (e.g., stable regulatory T cells that (a) comprise an exogenous human T cell receptor (TCR) that binds specifically to a target peptide complexed with an MHC molecule and (b) comprise a hypomethylated TSDR at an endogenous FOXP3 locus) is 1×10² to 1×10¹⁰, 1×10³ to 1×10¹⁰, 1×10⁴ to 1×10¹⁰, 1×10⁵ to 1×10¹⁰, 1×10⁶ to 1×10¹⁰, 1×10⁷ to 1×10¹⁰, 1×10⁸ to 1×10¹⁰, 1×10⁹ to 1×10¹⁰ stable regulatory T cells.

In some embodiments, the isolated cell populations (e.g., intended to be used in a pharmaceutical composition) are cryopreserved (e.g., subjected to one or more cryopreservation freeze-thaw cycles). That is, the isolated cell populations produced herein may be combined with a cryoprotecting agent, which lowers the melting temperature by forming chemical bonds with water and increasing the total concentration of solutes in the system. Non-limiting examples of cryoprotecting agents include glycerol, dimethyl sulfoxide (DMSO), ethanediol, and propanediol. While traditional methods that often include the use of serum and DMSO may be used, the disclosure also contemplates the use of cryopreservation medium manufactured under cGMP conditions and formulated serum-free and of non-animal origin (e.g., using <10% DMSO in the freeze cocktail). Other cryopreservation techniques are also provided herein, including more advanced techniques of cooling, for example, vitrifying cells without the use of cryoprotecting agents. See, e.g., Shinshu University. “A new way to ‘freeze’ cells promises to transform the common cell-freezing practice.” ScienceDaily.com April 2019. This process of ultrarapid cooling, utilizes inkjet cell printing to cool at a rate of 10,000 degrees Celsius/second, causing near-vitrification of the cells.

In some embodiments, the pharmaceutical composition comprises a population of stable regulatory T cells (e.g., stable regulatory T cells that (a) comprise an exogenous human T cell receptor (TCR) that binds specifically to a target peptide complexed with an MHC molecule and (b) comprise a hypomethylated TSDR at an endogenous FOXP3 locus) formulated with a cryoprotecting agent. In some embodiments, the cryoprotecting agent is a component of a cryopreservation medium (e.g., a cryopreservation medium containing 5% DMSO). Thus, in some embodiments, the pharmaceutical composition comprises a population of stable regulatory T cells (e.g., stable regulatory T cells that (a) comprise an exogenous human TCR that binds specifically to a target peptide complexed with an MHC molecule and (b) comprise a hypomethylated TSDR at an endogenous FOXP3 locus) formulated in a cryopreservation medium (e.g., a cryopreservation medium containing 5% DMSO). In some embodiments, a cryopreservation medium comprises 1-10%, 2-10%, 3-8%, 2-6%, 4-8%, or 4-6% DMSO. In some embodiments, a cryopreservation medium comprises 1%, 2%, 3%, 4%, 5%, 6%, 7%, 8%, 9%, or 10% DMSO.

### Methods of Administration

In some embodiments, the disclosure provides methods of administration of TCRs as described herein (and related pharmaceutical compositions) to a subject (e.g., a subject having Type 1 Diabetes). In some embodiments, the disclosure provides methods of administration of isolated cell populations comprising regulatory T cells as described herein (and related pharmaceutical compositions) to a subject (e.g., a subject having Type 1 Diabetes). Type 1 Diabetes, once known as juvenile diabetes or insulin-dependent diabetes, is a chronic condition. In this condition, the pancreas makes little or no insulin. Insulin is a hormone the body uses to allow sugar (glucose) to enter cells to produce energy. Different factors, such as genetics and some viruses, may cause Type 1 Diabetes. Although Type 1 Diabetes usually appears during childhood or adolescence, it can develop in adults. Even after much research, Type 1 Diabetes has no cure. Treatment is directed toward managing the amount of sugar in the blood using insulin, diet and lifestyle to prevent complications.

The natural progression of Type 1 Diabetes in a subject includes three stages. Stage 1 is characterized by normoglycemia accompanied by loss of self-tolerance and the development of two or more islet-directed autoantibodies. It is estimated that beta cells are 100% functional at this stage. The risk to develop Stage 3 Type 1 Diabetes in a Stage 1 subject is 44% after 5 years, 70% after 10 years and 100% over the lifetime of that subject. Stage 2 is characterized by accompaniment of the autoantibodies by dysglycemia, which reflects inadequate insulin secretion after glucose challenge. Stage 2 is typically asymptomatic despite an estimated 20-50% reduction in functional beta cells via immune destruction. The risk to develop Stage 3 Type 1 Diabetes in a Stage 1 subject is 75% after 5 years and 100% over the lifetime of that subject. Clinical diagnosis occurs at Stage 3. Stage 3 is characterized by the onset of symptoms and the need for insulin therapy. Approximately 20% of beta cells remain functional at Stage 3. The residual beta cell function results from variable insulitis (inflammation of the islets of Langerhans) that spares beta cell mass.

In some embodiments, the disclosure provides methods of administration of isolated cell populations comprising regulatory T cells as described herein (and related pharmaceutical compositions) to a subject having Stage 3 Type I Diabetes. In some embodiments, methods of administration of isolated cell populations comprising regulatory T cells as described herein (and related pharmaceutical compositions) to a subject target insulitis in the pancreas of the subject.

In some embodiments, the disclosure provides methods of administering cell population as described herein, e.g., a cell population comprising regulatory T cells transfected with a nucleic acid expressing an exogenous human TCR (e.g., a TCR as described in Table 1) that binds specifically to a GAD65 peptide (e.g., GAD65 555-567) complexed with an MHC or a pharmaceutical composition as described herein to a subject in an effective amount to alleviate one or more symptom of Type 1 Diabetes. In some embodiments, the disclosure provides methods of administering a cell population comprising regulatory T cells or a pharmaceutical composition as described herein to a subject in an effective amount to alleviate one or more symptom of Type 1 Diabetes. Non-limiting examples of symptoms of Type 1 Diabetes include reduced or no production of insulin, build-up of glucose/sugar in the bloodstream, feeling more thirsty than usual, urinating a lot, bed-wetting in children who have never wet the bed during the night, feeling very hungry, losing weight without trying, feeling irritable or having other mood changes, feeling tired and weak, having blurry vision.

In some embodiments, the disclosure provides methods of treating Type 1 Diabetes in a subject comprising administering a cell population comprising regulatory T cells or a pharmaceutical composition as described herein to the subject. In some embodiments, at least a portion of the population of cells are autologous cells (i.e., obtained from the same subject to which they are subsequently administered). In some embodiments, a population of cells is isolated from a subject, subjected to a method of producing a cell population (e.g., to increase the relative concentration of regulatory T cells within the population) as described herein, engineered to express an exogenous TCR, and then administered to the same subject in order to treat a disease.

In some embodiments, treating (or treatment of) a disease refers to a clinical intervention aimed to reverse, alleviate, delay the onset of, or inhibit the progress of Type 1 Diabetes, or one or more symptoms thereof. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., because of knowledge of genetic factors). Treatment may also be continued after symptoms have resolved, for example, to prevent or delay their recurrence.

A subject refers to an individual organism, for example, an individual human. In some embodiments, a subject is a human subject, such as a male subject or a female subject. In some embodiments, a human subject is an adult (e.g., 18 years of age or older). In some embodiments, a human subject is a juvenile (e.g., under 18 years of age). In some embodiments, a subject is a non-human mammal. In some embodiments, a subject is a non-human primate. In some embodiments, a subject is a rodent. In some embodiments, a subject is a sheep, a goat, a cow/cattle, a cat, or a dog. In some embodiments, a subject is a research animal. In some embodiments, a subject is genetically engineered, e.g., a genetically engineered non-human subject. A subject may be male or female.

A subject may have Type 1 Diabetes. In some embodiments, a subject has new-onset/adult-onset Type 1 Diabetes (e.g., new onset Stage 3 Type 1 Diabetes). A subject may have an HLA-DRB1*04:01 genetic haplotype. In some embodiments, a subject has an HLA-DRB1*04:01 genetic haplotype and has Type 1 Diabetes (e.g., new onset Stage 3 Type 1 Diabetes).

In some embodiments, a subject has residual beta cell function. In some embodiments, 5-50% of the beta cells within a subject are functional. In some embodiments, 5-40%, 5-30%, 5-25%, 10-50%, 10-40%, 10-30%, 15-30%, 15-25%, 15-20%, or 20-25% of the beta cells within a subject are functional. In some embodiments, about 5%, about 10%, about 15%, about 20%, about 25%, about 30%, about 35%, or about 40% of the beta cells within a subject are functional. A beta cell is considered functional if the beta cell secretes insulin and/or C-peptide.

In some embodiments, a subject has residual beta cell function if the C-peptide concentration in the plasma or blood of the subject is at least 300 μmol/L, 400 μmol/L, 450 pmol/L, 500 μmol/L, 550 μmol/L, 600 μmol/L, 700 μmol/L, 800 μmol/L, or 1000 μmol/L. In some embodiments, a subject has residual beta cell function if the C-peptide concentration in the plasma or blood of the subject is at least 0.1 ng/mL, 0.2 ng/ml, 0.3 ng/mL, 0.4 ng/mL, 0.5 ng/mL, 0.6 ng/ml, 0.7 ng/ml, 0.8 ng/mL, 0.9 ng/ml or 1 ng/mL. In some embodiments, a clinically relevant definition of residual beta cell function (e.g., contributing to glycemic control) is as described in Bonfanti R et al., Acta Diabetol 1998; 35:91-95.; or Palmer, J P et. al., Diabetes. 2004; 53:250-264; the contents of each of which are incorporated herein by reference.

In some embodiments, a subject does not have residual beta cell function. In some embodiments, endogenous insulin production by a subject is evidenced by circulating C-peptide levels, which are also a measure of preserved beta cell mass.

Conventional and pharmaceutically acceptable routes of administration of the include, but are not limited to, intravenous, subcutaneous, intravenous, intrathecal administration direct delivery to the selected organ (e.g., intraportal delivery to the liver), oral, inhalation (including intranasal and intratracheal delivery), intraocular, intramuscular, intradermal, intratumoral, and other parental routes of administration. Routes of administration may be combined, if desired.

A cell population comprising regulatory T cells or a pharmaceutical composition may be administered as a bolus administration. In some embodiments, administration of a cell population comprising regulatory T cells or a pharmaceutical composition comprises one or more infusion of the isolated cell population or pharmaceutical composition to the subject (e.g., cells are infused through a central line, similar to a blood transfusion).

### ADDITIONAL EMBODIMENTS

Additional embodiments of the disclosure are described in the numbered paragraphs below:

1. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 1,
a CDR2-alpha sequence comprising the amino acid sequence of SEQ ID NO: 2,
a CDR3-alpha sequence comprising the amino acid sequence of SEQ ID NO: 3,
a CDR1-beta sequence comprising the amino acid sequence of SEQ ID NO: 6,
a CDR2-beta sequence comprising the amino acid sequence of SEQ ID NO: 7, and/or
a CDR3-beta sequence comprising the amino acid sequence of SEQ ID NO: 8.
   2. The TCR of paragraph 1 comprising:
a CDR1-alpha sequence comprising the amino acid sequence of SEQ ID NO: 1,
a CDR2-alpha sequence comprising the amino acid sequence of SEQ ID NO: 2, and
a CDR3-alpha sequence comprising the amino acid sequence of SEQ ID NO: 3.
   3. The TCR of paragraph 1 or 2, wherein the alpha chain comprises an alpha variable region comprising an amino acid sequence having at least 90%, 95%, or 100% identity to the amino acid sequence of SEQ ID NO: 4.
   4. The TCR of paragraph 3, wherein the alpha chain comprises an amino acid sequence having at least 90%, 95%, or 100% identity to the amino acid sequence of SEQ ID NO: 5.
   5. The TCR of paragraph 3, wherein the alpha chain comprises an amino acid sequence having at least 90%, 95%, or 100% identity to the amino acid sequence of SEQ ID NO: 260.
   6. The TCR of any one of the preceding paragraphs comprising:
a CDR1-beta sequence comprising the amino acid sequence of SEQ ID NO: 6,
a CDR2-beta sequence comprising the amino acid sequence of SEQ ID NO: 7, and
a CDR3-beta sequence comprising the amino acid sequence of SEQ ID NO: 8.
   7. The TCR of paragraph 6, wherein the beta chain comprises a beta variable region comprising an amino acid sequence having at least 90%, 95%, or 100% identity to the amino acid sequence of SEQ ID NO: 9.
   8. The TCR of paragraph 7, wherein the beta chain comprises an amino acid sequence having at least 90%, 95%, or 100% identity to the amino acid sequence of SEQ ID NO: 10.
   9. The TCR of paragraph 7, wherein the beta chain comprises an amino acid sequence having at least 90%, 95%, or 100% identity to the amino acid sequence of SEQ ID NO: 261.
   10. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 11,
a CDR2-alpha sequence comprising the amino acid sequence of SEQ ID NO: 12,
a CDR3-alpha sequence comprising the amino acid sequence of SEQ ID NO: 13,
a CDR1-beta sequence comprising the amino acid sequence of SEQ ID NO: 16,
a CDR2-beta sequence comprising the amino acid sequence of SEQ ID NO: 17, and/or
a CDR3-beta sequence comprising the amino acid sequence of SEQ ID NO: 18.
   11. The TCR of paragraph 10 comprising:
a CDR1-alpha sequence comprising the amino acid sequence of SEQ ID NO: 11,
a CDR2-alpha sequence comprising the amino acid sequence of SEQ ID NO: 12, and
a CDR3-alpha sequence comprising the amino acid sequence of SEQ ID NO: 13.
   12. The TCR of paragraph 10 or 11, wherein the alpha chain comprises an alpha variable region comprising an amino acid sequence having at least 90%, 95%, or 100% identity to the amino acid sequence of SEQ ID NO: 14.
   13. The TCR of paragraph 12, wherein the alpha chain comprises an amino acid sequence having at least 90%, 95%, or 100% identity to the amino acid sequence of SEQ ID NO: 15.
   14. The TCR of paragraph 12, wherein the alpha chain comprises an amino acid sequence having at least 90%, 95%, or 100% identity to the amino acid sequence of SEQ ID NO: 262.
   15. The TCR of any one of paragraphs 10⁻¹⁴ comprising:
a CDR1-beta sequence comprising the amino acid sequence of SEQ ID NO: 16,
a CDR2-beta sequence comprising the amino acid sequence of SEQ ID NO: 17, and
a CDR3-beta sequence comprising the amino acid sequence of SEQ ID NO: 18.
   16. The TCR of paragraph 15, wherein the beta chain comprises an alpha variable region comprising an amino acid sequence having at least 90%, 95%, or 100% identity to the amino acid sequence of SEQ ID NO: 19.
   17. The TCR of paragraph 16, wherein the beta chain comprises an amino acid sequence having at least 90%, 95%, or 100% identity to the amino acid sequence of SEQ ID NO: 20.
   18. The TCR of paragraph 16, wherein the beta chain comprises an amino acid sequence having at least 90%, 95%, or 100% identity to the amino acid sequence of SEQ ID NO: 263.
   19. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 21,
a CDR2-alpha sequence comprising the amino acid sequence of SEQ ID NO: 22,
a CDR3-alpha sequence comprising the amino acid sequence of SEQ ID NO: 23,
a CDR1-beta sequence comprising the amino acid sequence of SEQ ID NO: 26,
a CDR2-beta sequence comprising the amino acid sequence of SEQ ID NO: 27, and/or
a CDR3-beta sequence comprising the amino acid sequence of SEQ ID NO: 28.
   20. The TCR of paragraph 19 comprising:
a CDR1-alpha sequence comprising the amino acid sequence of SEQ ID NO: 21,
a CDR2-alpha sequence comprising the amino acid sequence of SEQ ID NO: 22, and
a CDR3-alpha sequence comprising the amino acid sequence of SEQ ID NO: 23.
   21. The TCR of paragraph 19 or 20, wherein the alpha chain comprises an alpha variable region comprising an amino acid sequence having at least 90%, 95%, or 100% identity to the amino acid sequence of SEQ ID NO: 24.
   22. The TCR of paragraph 21, wherein the alpha chain comprises an amino acid sequence having at least 90%, 95%, or 100% identity to the amino acid sequence of SEQ ID NO: 25.
   23. The TCR of paragraph 21, wherein the alpha chain comprises an amino acid sequence having at least 90%, 95%, or 100% identity to the amino acid sequence of SEQ ID NO: 264.
   24. The TCR of any one of paragraphs 19-23 comprising:
a CDR1-beta sequence comprising the amino acid sequence of SEQ ID NO: 26,
a CDR2-beta sequence comprising the amino acid sequence of SEQ ID NO: 27, and
a CDR3-beta sequence comprising the amino acid sequence of SEQ ID NO: 28.
   25. The TCR of paragraph 24, wherein the beta chain comprises a beta variable region comprising an amino acid sequence having at least 90%, 95%, or 100% identity to the amino acid sequence of SEQ ID NO: 29.
   26. The TCR of paragraph 25, wherein the beta chain comprises an amino acid sequence having at least 90%, 95%, or 100% identity to the amino acid sequence of SEQ ID NO: 30.
   27. The TCR of paragraph 25, wherein the beta chain comprises an amino acid sequence having at least 90%, 95%, or 100% identity to the amino acid sequence of SEQ ID NO: 265.
   28. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 31,
a CDR2-alpha sequence comprising the amino acid sequence of SEQ ID NO: 32,
a CDR3-alpha sequence comprising the amino acid sequence of SEQ ID NO: 33,
a CDR1-beta sequence comprising the amino acid sequence of SEQ ID NO: 36,
a CDR2-beta sequence comprising the amino acid sequence of SEQ ID NO: 37, and/or
a CDR3-beta sequence comprising the amino acid sequence of SEQ ID NO: 38.
   29. The TCR of paragraph 28 comprising:
a CDR1-alpha sequence comprising the amino acid sequence of SEQ ID NO: 31,
a CDR2-alpha sequence comprising the amino acid sequence of SEQ ID NO: 32, and
a CDR3-alpha sequence comprising the amino acid sequence of SEQ ID NO: 33.
   30. The TCR of paragraph 28 or 29, wherein the alpha chain comprises an alpha variable region comprising an amino acid sequence having at least 90%, 95%, or 100% identity to the amino acid sequence of SEQ ID NO: 34.
   31. The TCR of paragraph 30, wherein the alpha chain comprises an amino acid sequence having at least 90%, 95%, or 100% identity to the amino acid sequence of SEQ ID NO: 35.
   32. The TCR of paragraph 30, wherein the alpha chain comprises an amino acid sequence having at least 90%, 95%, or 100% identity to the amino acid sequence of SEQ ID NO: 266.
   33. The TCR of any one of paragraphs 28-32 comprising:
a CDR1-beta sequence comprising the amino acid sequence of SEQ ID NO: 36,
a CDR2-beta sequence comprising the amino acid sequence of SEQ ID NO: 37, and
a CDR3-beta sequence comprising the amino acid sequence of SEQ ID NO: 38.
   34. The TCR of paragraph 33, wherein the beta chain comprises a beta variable region comprising an amino acid sequence having at least 90%, 95%, or 100% identity to the amino acid sequence of SEQ ID NO: 39.
   35. The TCR of paragraph 34, wherein the beta chain comprises an amino acid sequence having at least 90%, 95%, or 100% identity to the amino acid sequence of SEQ ID NO: 40.
   36. The TCR of paragraph 34, wherein the beta chain comprises an amino acid sequence having at least 90%, 95%, or 100% identity to the amino acid sequence of SEQ ID NO: 267.
   37. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 41,
a CDR2-alpha sequence comprising the amino acid sequence of SEQ ID NO: 42,
a CDR3-alpha sequence comprising the amino acid sequence of SEQ ID NO: 43,
a CDR1-beta sequence comprising the amino acid sequence of SEQ ID NO: 46,
a CDR2-beta sequence comprising the amino acid sequence of SEQ ID NO: 47, and/or
a CDR3-beta sequence comprising the amino acid sequence of SEQ ID NO: 48.
   38. The TCR of paragraph 37 comprising:
a CDR1-alpha sequence comprising the amino acid sequence of SEQ ID NO: 41,
a CDR2-alpha sequence comprising the amino acid sequence of SEQ ID NO: 42, and
a CDR3-alpha sequence comprising the amino acid sequence of SEQ ID NO: 43.
   39. The TCR of paragraph 37 or 38, wherein the alpha chain comprises an alpha variable region comprising an amino acid sequence having at least 90%, 95%, or 100% identity to the amino acid sequence of SEQ ID NO: 44.
   40. The TCR of paragraph 39, wherein the alpha chain comprises an amino acid sequence having at least 90%, 95%, or 100% identity to the amino acid sequence of SEQ ID NO: 45.
   41. The TCR of paragraph 39, wherein the alpha chain comprises an amino acid sequence having at least 90%, 95%, or 100% identity to the amino acid sequence of SEQ ID NO: 268.
   42. The TCR of any one of paragraphs 37-41 comprising:
a CDR1-beta sequence comprising the amino acid sequence of SEQ ID NO: 46,
a CDR2-beta sequence comprising the amino acid sequence of SEQ ID NO: 47, and
a CDR3-beta sequence comprising the amino acid sequence of SEQ ID NO: 48.
   43. The TCR of paragraph 42, wherein the beta chain comprises a beta variable region comprising an amino acid sequence having at least 90%, 95%, or 100% identity to the amino acid sequence of SEQ ID NO: 49.
   44. The TCR of paragraph 43, wherein the beta chain comprises an amino acid sequence having at least 90%, 95%, or 100% identity to the amino acid sequence of SEQ ID NO: 50.
   45. The TCR of paragraph 43, wherein the beta chain comprises an amino acid sequence having at least 90%, 95%, or 100% identity to the amino acid sequence of SEQ ID NO: 269.
   46. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 51,
a CDR2-alpha sequence comprising the amino acid sequence of SEQ ID NO: 52,
a CDR3-alpha sequence comprising the amino acid sequence of SEQ ID NO: 53,
a CDR1-beta sequence comprising the amino acid sequence of SEQ ID NO: 56,
a CDR2-beta sequence comprising the amino acid sequence of SEQ ID NO: 57, and/or
a CDR3-beta sequence comprising the amino acid sequence of SEQ ID NO: 58.
   47. The TCR of paragraph 46 comprising:
a CDR1-alpha sequence comprising the amino acid sequence of SEQ ID NO: 51,
a CDR2-alpha sequence comprising the amino acid sequence of SEQ ID NO: 52, and
a CDR3-alpha sequence comprising the amino acid sequence of SEQ ID NO: 53.
   48. The TCR of paragraph 46 or 47, wherein the alpha chain comprises an alpha variable region comprising an amino acid sequence having at least 90%, 95%, or 100% identity to the amino acid sequence of SEQ ID NO: 54.
   49. The TCR of paragraph 48, wherein the alpha chain comprises an amino acid sequence having at least 90%, 95%, or 100% identity to the amino acid sequence of SEQ ID NO: 55.
   50. The TCR of paragraph 48, wherein the alpha chain comprises an amino acid sequence having at least 90%, 95%, or 100% identity to the amino acid sequence of SEQ ID NO: 270.
   51. The TCR of any one of paragraphs 46-50 comprising:
a CDR1-beta sequence comprising the amino acid sequence of SEQ ID NO: 56,
a CDR2-beta sequence comprising the amino acid sequence of SEQ ID NO: 57, and
a CDR3-beta sequence comprising the amino acid sequence of SEQ ID NO: 58.
   52. The TCR of paragraph 51, wherein the beta chain comprises a beta variable region comprising an amino acid sequence having at least 90%, 95%, or 100% identity to the amino acid sequence of SEQ ID NO: 59.
   53. The TCR of paragraph 52, wherein the beta chain comprises an amino acid sequence having at least 90%, 95%, or 100% identity to the amino acid sequence of SEQ ID NO: 60.
   54. The TCR of paragraph 52, wherein the beta chain comprises an amino acid sequence having at least 90%, 95%, or 100% identity to the amino acid sequence of SEQ ID NO: 271.
   55. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 61,
a CDR2-alpha sequence comprising the amino acid sequence of SEQ ID NO: 62,
a CDR3-alpha sequence comprising the amino acid sequence of SEQ ID NO: 63,
a CDR1-beta sequence comprising the amino acid sequence of SEQ ID NO: 66,
a CDR2-beta sequence comprising the amino acid sequence of SEQ ID NO: 67, and/or
   56. The TCR of paragraph 55 comprising:
a CDR1-alpha sequence comprising the amino acid sequence of SEQ ID NO: 61,
a CDR2-alpha sequence comprising the amino acid sequence of SEQ ID NO: 62, and
a CDR3-alpha sequence comprising the amino acid sequence of SEQ ID NO: 63.
   57. The TCR of paragraph 55 or 56, wherein the alpha chain comprises an alpha variable region comprising an amino acid sequence having at least 90%, 95%, or 100% identity to the amino acid sequence of SEQ ID NO: 64.
   58. The TCR of paragraph 57, wherein the alpha chain comprises an amino acid sequence having at least 90%, 95%, or 100% identity to the amino acid sequence of SEQ ID NO: 65.
   59. The TCR of paragraph 57, wherein the alpha chain comprises an amino acid sequence having at least 90%, 95%, or 100% identity to the amino acid sequence of SEQ ID NO: 272.
   60. The TCR of any one of paragraphs 55-59 comprising:
a CDR1-beta sequence comprising the amino acid sequence of SEQ ID NO: 66,
a CDR2-beta sequence comprising the amino acid sequence of SEQ ID NO: 67, and
a CDR3-beta sequence comprising the amino acid sequence of SEQ ID NO: 68.
   61. The TCR of paragraph 60, wherein the beta chain comprises a beta variable region comprising an amino acid sequence having at least 90%, 95%, or 100% identity to the amino acid sequence of SEQ ID NO: 69.
   62. The TCR of paragraph 61, wherein the beta chain comprises an amino acid sequence having at least 90%, 95%, or 100% identity to the amino acid sequence of SEQ ID NO: 70.
   63. The TCR of paragraph 61, wherein the beta chain comprises an amino acid sequence having at least 90%, 95%, or 100% identity to the amino acid sequence of SEQ ID NO: 273.
   64. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 71,
a CDR2-alpha sequence comprising the amino acid sequence of SEQ ID NO: 72,
a CDR3-alpha sequence comprising the amino acid sequence of SEQ ID NO: 73,
a CDR1-beta sequence comprising the amino acid sequence of SEQ ID NO: 76,
a CDR2-beta sequence comprising the amino acid sequence of SEQ ID NO: 77, and/or
a CDR3-beta sequence comprising the amino acid sequence of SEQ ID NO: 78.
   65. The TCR of paragraph 64 comprising:
a CDR1-alpha sequence comprising the amino acid sequence of SEQ ID NO: 71,
a CDR2-alpha sequence comprising the amino acid sequence of SEQ ID NO: 72, and
a CDR3-alpha sequence comprising the amino acid sequence of SEQ ID NO: 73.
   66. The TCR of paragraph 64 or 65, wherein the alpha chain comprises an alpha variable region comprising an amino acid sequence having at least 90%, 95%, or 100% identity to the amino acid sequence of SEQ ID NO: 74.
   67. The TCR of paragraph 66, wherein the alpha chain comprises an amino acid sequence having at least 90%, 95%, or 100% identity to the amino acid sequence of SEQ ID NO: 75.
   68. The TCR of paragraph 66, wherein the alpha chain comprises an amino acid sequence having at least 90%, 95%, or 100% identity to the amino acid sequence of SEQ ID NO: 274.
   69. The TCR of any one of paragraphs 64-68 comprising:
a CDR1-beta sequence comprising the amino acid sequence of SEQ ID NO: 76,
a CDR2-beta sequence comprising the amino acid sequence of SEQ ID NO: 77, and
a CDR3-beta sequence comprising the amino acid sequence of SEQ ID NO: 78.
   70. The TCR of paragraph 69, wherein the beta chain comprises a beta variable region comprising an amino acid sequence having at least 90%, 95%, or 100% identity to the amino acid sequence of SEQ ID NO: 79.
   71. The TCR of paragraph 70, wherein the beta chain comprises an amino acid sequence having at least 90%, 95%, or 100% identity to the amino acid sequence of SEQ ID NO: 80.
   72. The TCR of paragraph 70, wherein the beta chain comprises an amino acid sequence having at least 90%, 95%, or 100% identity to the amino acid sequence of SEQ ID NO: 275.
   73. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 81,
a CDR2-alpha sequence comprising the amino acid sequence of SEQ ID NO: 82,
a CDR3-alpha sequence comprising the amino acid sequence of SEQ ID NO: 83,
a CDR1-beta sequence comprising the amino acid sequence of SEQ ID NO: 86,
a CDR2-beta sequence comprising the amino acid sequence of SEQ ID NO: 87, and/or
a CDR3-beta sequence comprising the amino acid sequence of SEQ ID NO: 88.
   74. The TCR of paragraph 73 comprising:
a CDR1-alpha sequence comprising the amino acid sequence of SEQ ID NO: 81,
a CDR2-alpha sequence comprising the amino acid sequence of SEQ ID NO: 82, and
a CDR3-alpha sequence comprising the amino acid sequence of SEQ ID NO: 83.
   75. The TCR of paragraph 73 or 74, wherein the alpha chain comprises an alpha variable region comprising an amino acid sequence having at least 90%, 95%, or 100% identity to the amino acid sequence of SEQ ID NO: 84.
   76. The TCR of paragraph 75, wherein the alpha chain comprises an amino acid sequence having at least 90%, 95%, or 100% identity to the amino acid sequence of SEQ ID NO: 85.
   77. The TCR of paragraph 75, wherein the alpha chain comprises an amino acid sequence having at least 90%, 95%, or 100% identity to the amino acid sequence of SEQ ID NO: 276.
   78. The TCR of any one of paragraphs 73-77 comprising:
a CDR1-beta sequence comprising the amino acid sequence of SEQ ID NO: 86,
a CDR2-beta sequence comprising the amino acid sequence of SEQ ID NO: 87, and
a CDR3-beta sequence comprising the amino acid sequence of SEQ ID NO: 88.
   79. The TCR of paragraph 78, wherein the beta chain comprises a beta variable region comprising an amino acid sequence having at least 90%, 95%, or 100% identity to the amino acid sequence of SEQ ID NO: 89.
   80. The TCR of paragraph 79, wherein the beta chain comprises an amino acid sequence having at least 90%, 95%, or 100% identity to the amino acid sequence of SEQ ID NO: 90.
   81. The TCR of paragraph 79, wherein the beta chain comprises an amino acid sequence having at least 90%, 95%, or 100% identity to the amino acid sequence of SEQ ID NO: 277.
   82. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 91,
a CDR2-alpha sequence comprising the amino acid sequence of SEQ ID NO: 92,
a CDR3-alpha sequence comprising the amino acid sequence of SEQ ID NO: 93,
a CDR1-beta sequence comprising the amino acid sequence of SEQ ID NO: 96,
a CDR2-beta sequence comprising the amino acid sequence of SEQ ID NO: 97, and/or
a CDR3-beta sequence comprising the amino acid sequence of SEQ ID NO: 98.
   83. The TCR of paragraph 82 comprising:
a CDR1-alpha sequence comprising the amino acid sequence of SEQ ID NO: 91,
a CDR2-alpha sequence comprising the amino acid sequence of SEQ ID NO: 92, and
a CDR3-alpha sequence comprising the amino acid sequence of SEQ ID NO: 93.
   84. The TCR of paragraph 82 or 83, wherein the alpha chain comprises an alpha variable region comprising an amino acid sequence having at least 90%, 95%, or 100% identity to the amino acid sequence of SEQ ID NO: 94.
   85. The TCR of paragraph 84, wherein the alpha chain comprises an amino acid sequence having at least 90%, 95%, or 100% identity to the amino acid sequence of SEQ ID NO: 95.
   86. The TCR of paragraph 84, wherein the alpha chain comprises an amino acid sequence having at least 90%, 95%, or 100% identity to the amino acid sequence of SEQ ID NO: 278.
   87. The TCR of any one of paragraphs 82-86 comprising:
a CDR1-beta sequence comprising the amino acid sequence of SEQ ID NO: 96,
a CDR2-beta sequence comprising the amino acid sequence of SEQ ID NO: 97, and
a CDR3-beta sequence comprising the amino acid sequence of SEQ ID NO: 98.
   88. The TCR of paragraph 87, wherein the beta chain comprises a beta variable region comprising an amino acid sequence having at least 90%, 95%, or 100% identity to the amino acid sequence of SEQ ID NO: 99.
   89. The TCR of paragraph 88, wherein the beta chain comprises an amino acid sequence having at least 90%, 95%, or 100% identity to the amino acid sequence of SEQ ID NO: 100.
   90. The TCR of paragraph 88, wherein the beta chain comprises an amino acid sequence having at least 90%, 95%, or 100% identity to the amino acid sequence of SEQ ID NO: 279.
   91. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 110,
a CDR2-alpha sequence comprising the amino acid sequence of SEQ ID NO: 111,
a CDR3-alpha sequence comprising the amino acid sequence of SEQ ID NO: 112,
a CDR1-beta sequence comprising the amino acid sequence of SEQ ID NO: 115,
a CDR2-beta sequence comprising the amino acid sequence of SEQ ID NO: 116, and/or
a CDR3-beta sequence comprising the amino acid sequence of SEQ ID NO: 117.
   92. The TCR of paragraph 91 comprising:
a CDR1-alpha sequence comprising the amino acid sequence of SEQ ID NO: 110,
a CDR2-alpha sequence comprising the amino acid sequence of SEQ ID NO: 111, and
a CDR3-alpha sequence comprising the amino acid sequence of SEQ ID NO: 112.
   93. The TCR of paragraph 91 or 92, wherein the alpha chain comprises an alpha variable region comprising an amino acid sequence having at least 90%, 95%, or 100% identity to the amino acid sequence of SEQ ID NO: 113.
   94. The TCR of paragraph 93, wherein the alpha chain comprises an amino acid sequence having at least 90%, 95%, or 100% identity to the amino acid sequence of SEQ ID NO: 114.
   95. The TCR of paragraph 93, wherein the alpha chain comprises an amino acid sequence having at least 90%, 95%, or 100% identity to the amino acid sequence of SEQ ID NO: 280.
   96. The TCR of any one of paragraphs 91-95 comprising:
a CDR1-beta sequence comprising the amino acid sequence of SEQ ID NO: 115,
a CDR2-beta sequence comprising the amino acid sequence of SEQ ID NO: 116, and
a CDR3-beta sequence comprising the amino acid sequence of SEQ ID NO: 117.
   97 The TCR of paragraph 96, wherein the beta chain comprises a beta variable region comprising an amino acid sequence having at least 90%, 95%, or 100% identity to the amino acid sequence of SEQ ID NO: 118.
   98. The TCR of paragraph 97, wherein the beta chain comprises an amino acid sequence having at least 90%, 95%, or 100% identity to the amino acid sequence of SEQ ID NO: 119.
   99. The TCR of paragraph 97, wherein the beta chain comprises an amino acid sequence having at least 90%, 95%, or 100% identity to the amino acid sequence of SEQ ID NO: 281.
   100. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 120,
a CDR2-alpha sequence comprising the amino acid sequence of SEQ ID NO: 121,
a CDR3-alpha sequence comprising the amino acid sequence of SEQ ID NO: 122,
a CDR1-beta sequence comprising the amino acid sequence of SEQ ID NO: 125,
a CDR2-beta sequence comprising the amino acid sequence of SEQ ID NO: 126, and/or
a CDR3-beta sequence comprising the amino acid sequence of SEQ ID NO: 127.
   101. The TCR of paragraph 100 comprising:
a CDR1-alpha sequence comprising the amino acid sequence of SEQ ID NO: 120,
a CDR2-alpha sequence comprising the amino acid sequence of SEQ ID NO: 121, and
a CDR3-alpha sequence comprising the amino acid sequence of SEQ ID NO: 122.
   102. The TCR of paragraph 100 or 101, wherein the alpha chain comprises an alpha variable region comprising an amino acid sequence having at least 90%, 95%, or 100% identity to the amino acid sequence of SEQ ID NO: 123.
   103. The TCR of paragraph 102, wherein the alpha chain comprises an amino acid sequence having at least 90%, 95%, or 100% identity to the amino acid sequence of SEQ ID NO: 124.
   104. The TCR of paragraph 102, wherein the alpha chain comprises an amino acid sequence having at least 90%, 95%, or 100% identity to the amino acid sequence of SEQ ID NO: 282.
   105. The TCR of any one of paragraphs 100-104 comprising:
a CDR1-beta sequence comprising the amino acid sequence of SEQ ID NO: 125,
a CDR2-beta sequence comprising the amino acid sequence of SEQ ID NO: 126, and
a CDR3-beta sequence comprising the amino acid sequence of SEQ ID NO: 127.
   106. The TCR of paragraph 105, wherein the beta chain comprises a beta variable region comprising an amino acid sequence having at least 90%, 95%, or 100% identity to the amino acid sequence of SEQ ID NO: 128.
   107. The TCR of paragraph 106, wherein the beta chain comprises an amino acid sequence having at least 90%, 95%, or 100% identity to the amino acid sequence of SEQ ID NO: 129.
   108. The TCR of paragraph 106, wherein the beta chain comprises an amino acid sequence having at least 90%, 95%, or 100% identity to the amino acid sequence of SEQ ID NO: 283.
   109. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 130,
a CDR2-alpha sequence comprising the amino acid sequence of SEQ ID NO: 131,
a CDR3-alpha sequence comprising the amino acid sequence of SEQ ID NO: 132,
a CDR1-beta sequence comprising the amino acid sequence of SEQ ID NO: 135,
a CDR2-beta sequence comprising the amino acid sequence of SEQ ID NO: 136, and/or
a CDR3-beta sequence comprising the amino acid sequence of SEQ ID NO: 137.
   110. The TCR of paragraph 109 comprising:
a CDR1-alpha sequence comprising the amino acid sequence of SEQ ID NO: 130,
a CDR2-alpha sequence comprising the amino acid sequence of SEQ ID NO: 131, and
a CDR3-alpha sequence comprising the amino acid sequence of SEQ ID NO: 132.
   111. The TCR of paragraph 110, wherein the alpha chain comprises an alpha variable region comprising an amino acid sequence having at least 90%, 95%, or 100% identity to the amino acid sequence of SEQ ID NO: 133.
   112. The TCR of paragraph 111, wherein the alpha chain comprises an amino acid sequence having at least 90%, 95%, or 100% identity to the amino acid sequence of SEQ ID NO: 134.
   113. The TCR of paragraph 111, wherein the alpha chain comprises an amino acid sequence having at least 90%, 95%, or 100% identity to the amino acid sequence of SEQ ID NO: 284.
   114. The TCR of any one of paragraphs 109-113 comprising:
a CDR1-beta sequence comprising the amino acid sequence of SEQ ID NO: 135,
a CDR2-beta sequence comprising the amino acid sequence of SEQ ID NO: 136, and
a CDR3-beta sequence comprising the amino acid sequence of SEQ ID NO: 137.
   115. The TCR of paragraph 114, wherein the beta chain comprises a beta variable region comprising an amino acid sequence having at least 90%, 95%, or 100% identity to the amino acid sequence of SEQ ID NO: 138.
   116. The TCR of paragraph 115, wherein the beta chain comprises an amino acid sequence having at least 90%, 95%, or 100% identity to the amino acid sequence of SEQ ID NO: 139.
   117. The TCR of paragraph 115, wherein the beta chain comprises an amino acid sequence having at least 90%, 95%, or 100% identity to the amino acid sequence of SEQ ID NO: 285.
   118. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 140,
a CDR2-alpha sequence comprising the amino acid sequence of SEQ ID NO: 141,
a CDR3-alpha sequence comprising the amino acid sequence of SEQ ID NO: 142,
a CDR1-beta sequence comprising the amino acid sequence of SEQ ID NO: 145,
a CDR2-beta sequence comprising the amino acid sequence of SEQ ID NO: 146, and/or
a CDR3-beta sequence comprising the amino acid sequence of SEQ ID NO: 147.
   119. The TCR of paragraph 118 comprising:
a CDR1-alpha sequence comprising the amino acid sequence of SEQ ID NO: 140,
a CDR2-alpha sequence comprising the amino acid sequence of SEQ ID NO: 141, and
a CDR3-alpha sequence comprising the amino acid sequence of SEQ ID NO: 142.
   120. The TCR of paragraph 119, wherein the alpha chain comprises an alpha variable region comprising an amino acid sequence having at least 90%, 95%, or 100% identity to the amino acid sequence of SEQ ID NO: 143.
   121. The TCR of paragraph 120, wherein the alpha chain comprises an amino acid sequence having at least 90%, 95%, or 100% identity to the amino acid sequence of SEQ ID NO: 144.
   122. The TCR of paragraph 120, wherein the alpha chain comprises an amino acid sequence having at least 90%, 95%, or 100% identity to the amino acid sequence of SEQ ID NO: 286.
   123. The TCR of any one of paragraphs 118-122 comprising:
a CDR1-beta sequence comprising the amino acid sequence of SEQ ID NO: 145,
a CDR2-beta sequence comprising the amino acid sequence of SEQ ID NO: 146, and
a CDR3-beta sequence comprising the amino acid sequence of SEQ ID NO: 147.
   124. The TCR of paragraph 123, wherein the beta chain comprises a beta variable region comprising an amino acid sequence having at least 90%, 95%, or 100% identity to the amino acid sequence of SEQ ID NO: 148.
   125. The TCR of paragraph 124, wherein the beta chain comprises an amino acid sequence having at least 90%, 95%, or 100% identity to the amino acid sequence of SEQ ID NO: 149.
   126. The TCR of paragraph 124, wherein the beta chain comprises an amino acid sequence having at least 90%, 95%, or 100% identity to the amino acid sequence of SEQ ID NO: 287.
   127. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 150,
a CDR2-alpha sequence comprising the amino acid sequence of SEQ ID NO: 151,
a CDR3-alpha sequence comprising the amino acid sequence of SEQ ID NO: 152,
a CDR1-beta sequence comprising the amino acid sequence of SEQ ID NO: 155,
a CDR2-beta sequence comprising the amino acid sequence of SEQ ID NO: 156, and/or
a CDR3-beta sequence comprising the amino acid sequence of SEQ ID NO: 157.
   128. The TCR of paragraph 127 comprising:
a CDR1-alpha sequence comprising the amino acid sequence of SEQ ID NO: 150,
a CDR2-alpha sequence comprising the amino acid sequence of SEQ ID NO: 151, and
a CDR3-alpha sequence comprising the amino acid sequence of SEQ ID NO: 152.
   129. The TCR of paragraph 128, wherein the alpha chain comprises an alpha variable region comprising an amino acid sequence having at least 90%, 95%, or 100% identity to the amino acid sequence of SEQ ID NO: 153.
   130. The TCR of paragraph 129, wherein the alpha chain comprises an amino acid sequence having at least 90%, 95%, or 100% identity to the amino acid sequence of SEQ ID NO: 154.
   131. The TCR of paragraph 129, wherein the alpha chain comprises an amino acid sequence having at least 90%, 95%, or 100% identity to the amino acid sequence of SEQ ID NO: 288.
   132. The TCR of any one of paragraphs 127-131 comprising:
a CDR1-beta sequence comprising the amino acid sequence of SEQ ID NO: 155,
a CDR2-beta sequence comprising the amino acid sequence of SEQ ID NO: 156, and
a CDR3-beta sequence comprising the amino acid sequence of SEQ ID NO: 157.
   133. The TCR of paragraph 132, wherein the beta chain comprises a beta variable region comprising an amino acid sequence having at least 90%, 95%, or 100% identity to the amino acid sequence of SEQ ID NO: 158.
   134. The TCR of paragraph 133, wherein the beta chain comprises an amino acid sequence having at least 90%, 95%, or 100% identity to the amino acid sequence of SEQ ID NO: 159.
   135. The TCR of paragraph 133, wherein the beta chain comprises an amino acid sequence having at least 90%, 95%, or 100% identity to the amino acid sequence of SEQ ID NO: 289.
   136. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 160,
a CDR2-alpha sequence comprising the amino acid sequence of SEQ ID NO: 161,
a CDR3-alpha sequence comprising the amino acid sequence of SEQ ID NO: 162,
a CDR1-beta sequence comprising the amino acid sequence of SEQ ID NO: 165,
a CDR2-beta sequence comprising the amino acid sequence of SEQ ID NO: 166, and/or
a CDR3-beta sequence comprising the amino acid sequence of SEQ ID NO: 167.
   137. The TCR of paragraph 136 comprising:
a CDR1-alpha sequence comprising the amino acid sequence of SEQ ID NO: 160,
a CDR2-alpha sequence comprising the amino acid sequence of SEQ ID NO: 161, and
a CDR3-alpha sequence comprising the amino acid sequence of SEQ ID NO: 162.
   138. The TCR of paragraph 137, wherein the alpha chain comprises an alpha variable region comprising an amino acid sequence having at least 90%, 95%, or 100% identity to the amino acid sequence of SEQ ID NO: 163.
   139. The TCR of paragraph 138, wherein the alpha chain comprises an amino acid sequence having at least 90%, 95%, or 100% identity to the amino acid sequence of SEQ ID NO: 164.
   140. The TCR of paragraph 138, wherein the alpha chain comprises an amino acid sequence having at least 90%, 95%, or 100% identity to the amino acid sequence of SEQ ID NO: 290.
   141. The TCR of any one of paragraphs 136-140 comprising:
a CDR1-beta sequence comprising the amino acid sequence of SEQ ID NO: 165,
a CDR2-beta sequence comprising the amino acid sequence of SEQ ID NO: 166, and
a CDR3-beta sequence comprising the amino acid sequence of SEQ ID NO: 167.
   142. The TCR of paragraph 141, wherein the beta chain comprises a beta variable region comprising an amino acid sequence having at least 90%, 95%, or 100% identity to the amino acid sequence of SEQ ID NO: 168.
   143. The TCR of paragraph 142, wherein the beta chain comprises an amino acid sequence having at least 90%, 95%, or 100% identity to the amino acid sequence of SEQ ID NO: 169.
   144. The TCR of paragraph 142, wherein the beta chain comprises an amino acid sequence having at least 90%, 95%, or 100% identity to the amino acid sequence of SEQ ID NO: 291.
   145. The TCR of any one of the preceding paragraphs, wherein the GAD65 peptide is a GAD65 555-567 peptide comprising the amino acid sequence of NFFRMVISNPAAT (SEQ ID NO: 109).
   146. The TCR of any one of the preceding paragraphs, wherein the MHC comprises HLA-DRB1*04:01.
   147. The TCR of any one of the preceding paragraphs, wherein the TCR is encoded as a single polypeptide, optionally wherein the polypeptide comprises an N-terminal beta domain and a C-terminal alpha domain.
   148. The TCR of paragraph 147, wherein the single polypeptide comprises a self-cleaving peptide sequence positioned between the alpha chain and the beta chain, optionally wherein the self-cleaving peptide sequence is a 2A peptide sequence, optionally wherein the 2A peptide sequence is a P2A, E2A, F2A, or T2A peptide sequence.
   149. The TCR of any one of the preceding paragraphs, wherein the alpha chain comprises an alpha constant region comprising an amino acid sequence having at least 90%, 95%, or 100% identity to the amino acid sequence of SEQ ID NO: 106.
   150. The TCR of any one of the preceding paragraphs, wherein the beta chain comprises a beta constant region comprising an amino acid sequence having at least 90%, 95%, or 100% identity to the amino acid sequence of SEQ ID NO: 107 or 108.
   151. The TCR of any one of the preceding paragraphs, wherein TCR comprises one or more amino acid substitutions to cysteine residues in the alpha chain constant region and the beta chain constant region, and wherein the cysteine residues are capable of forming one or more disulfide bridges.
   152. The TCR of any one of the preceding paragraphs, wherein the alpha chain constant region comprises a T48C amino acid substitution relative to an alpha chain constant region comprising the amino acid sequence of SEQ ID NO: 106, and wherein the beta chain constant region comprises a S57C amino acid substitution relative to a beta chain constant region comprising the amino acid sequence of SEQ ID NO: 108.
   153. An engineered nucleic acid encoding the TCR of any one of the preceding paragraphs.
   154. The engineered nucleic acid of paragraph 153, wherein the nucleic acid is a vector.
   155. The engineered nucleic acid of paragraph 154, wherein the vector is a viral vector.
   156. The engineered nucleic acid of paragraph 155, wherein the viral vector is a lentiviral vector.
   157. The engineered nucleic acid of any one of paragraphs 153-156, wherein the nucleic acid comprises a promoter operably linked to a coding sequence encoding the TCR, optionally wherein the promoter is an EF-1 alpha promoter or an MND promoter.
   158. The engineered nucleic acid of any one of paragraphs 153-157, wherein the nucleic acid further comprises an enhancer element, optionally an optimized post-transcriptional regulatory element (oPRE) or a woodchuck hepatitis virus post-transcriptional regulatory element (WPRE), further optionally WPRE-mut6.
   159. The engineered nucleic acid of any one of paragraphs 153-158, wherein the coding sequence is codon-optimized.
   160. An engineered cell comprising a TCR that specifically binds GAD65 555-567 complexed with a major histocompatibility complex (MHC) molecule, wherein the TCR is exogenous to the engineered cell.
   161. The engineered cell of paragraph 160, wherein the TCR comprises the TCR of any one of paragraphs 1-152 or the nucleic acid of any one of paragraphs 153-159.
   162. The engineered cell of paragraph 160 or 161, wherein the cell is a T cell, optionally a regulatory T cell.
   163. An engineered regulatory T cell comprising a TCR that specifically binds GAD65 555-567 peptide, complexed with a major histocompatibility complex (MHC) molecule, wherein the TCR is exogenous to the engineered cell.
   164. The engineered regulatory T cell of paragraph 163, wherein the TCR comprises the TCR of any one of paragraphs 1-152 or the nucleic acid of any one of paragraphs 153-159.
   165. The engineered cell of any one of paragraphs 160-164, wherein the cell is derived from a subject, optionally a subject having or suspected of having Type 1 Diabetes. 166. A cell population comprising a plurality of the engineered cell of any one of paragraphs 160-165.
   167. A pharmaceutical composition comprising the cell population of paragraph 166 and a pharmaceutically acceptable excipient.
   168. A composition comprising the cell population of paragraph 166 and a cryopreservative.
   169. A method comprising administering to a subject the cell population of paragraph 166, the pharmaceutical composition of paragraph 167, or the composition of paragraph 168, wherein the subject has Type 1 Diabetes.
   170. The method of paragraph 169, wherein the cell population or the pharmaceutical composition, or the composition is administered in an effective amount to alleviate one or more symptom of Type 1 Diabetes.
   171. The method of paragraph 169 or 170, wherein the administering comprises intravenous administration.
   172. The method of any one of paragraphs 169-171, wherein the administering comprises one or more infusion.

### EXAMPLES

### Example 1. TCR Characterization

T-cell receptors that are specific to GAD65 555-567 (SEQ ID NO: 109; NFFRMVISNPAAT) were identified by repeated stimulation of autologously matched CD4⁺ T cells and monocytes isolated from peripheral blood mononuclear cells (PBMCs) of a healthy HLA-DRB1*04:01 donor leukopak. 24 TCRs of clonal population of cells that expanded in response to antigen were selected for further analysis.

TCRs selected for further analysis were cloned into a lentiviral vector. Each of the TCRs was encoded by lentiviral vector constructs that comprised an N-terminal TCR beta chain and a C-terminal TCR alpha chain with a linker domain comprising a GSG amino acid sequence followed by a P2A self-cleaving peptide. The TCR beta-GSG-P2A-TCR alpha fusion constructs were expressed under the EF1a promoter. Additionally, a vector encoding the R164 TCR was included as a positive control (“Control TCR”). In some instances, the TCR sequences were engineered to introduce an additional cysteine bridge between the alpha and beta constant regions (DS, or disulfide modified).

The vectors were transduced into TCR-null Jurkat cells by electroporation and expression was confirmed via flow cytometry. The ability of the TCRs to activate Jurkat cells in response to antigen (GAD65 555-567) stimulation was tested. TCRs tested were either unmodified or disulfide modified. One day following transfection, Jurkat cells were co-cultured at a 1:1 ratio with BLS-DRB1*04:01 cells (HLA-DR monogenic B cell line) and 10 μM GAD65 555-567 peptide. Flow cytometry was used to measure upregulation of CD69 surface expression. 14 of the 24 TCRs tested demonstrated antigen-specific activation.

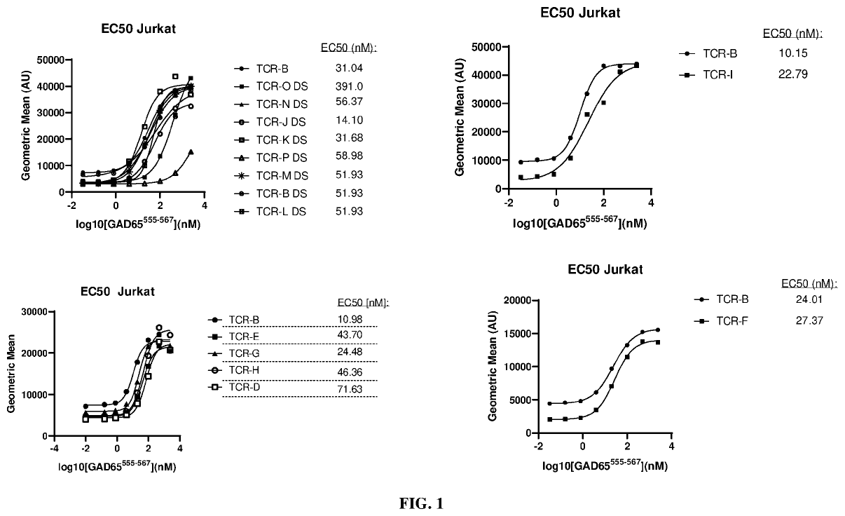

EC50 curves for these 14 TCRs were generated. TCRs tested were either unmodified, disulfide modified, or both versions were tested. Three days following transfection, Jurkat cells were co-cultured at a 1:1 ratio with BLS-DRB1*04:01 cells (HLA-DR monogenic B cell line) and GAD65 555-567 peptide in a 7-pt dose response curve from 2.5 μM peptide. Flow cytometry was used to measure surface level expression of CD69 following this co-culture. Representative EC50 curves are shown in FIG. 1.

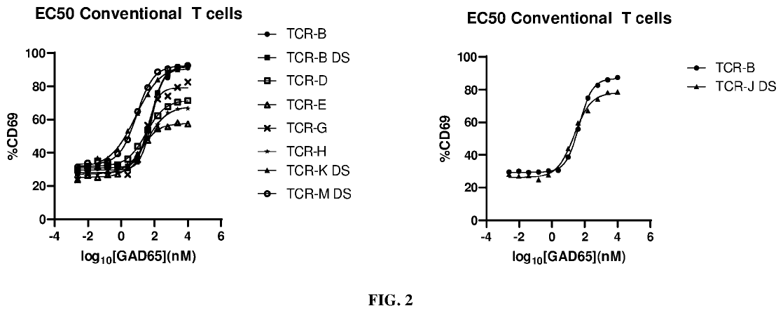

9 of the 14 TCRs were carried forward for further screening, in conventional T cells (which are expressing endogenous TCRs). Expression of TCRs was confirmed via flow cytometry. TCRs tested were either unmodified, disulfide modified, or both versions were tested. Conventional CD4⁺ T cells were isolated from peripheral blood mononuclear cells (PBMC) of healthy HLA-DRB1*04:01+ donors. Conventional T cells were activated in a 3:1 ratio with anti-CD3/CD28 beads (Dyna beads; ThermoFisher). After 24 hours, beads were removed from the cells and cells were transduced with lentivirus expressing the GAD65-specific TCRs in the presence of IL-7 and IL-2. Four days following transduction, CD4⁺ T cells were co-cultured with BLS-DRB1*04:01 cells at a 1:2 ratio BLS-DRB1*04:01 cells to conventional CD4+ T cells in the presence of varying concentrations of GAD65 555-567 peptide (an 11-pt dose response curve from 10 μM peptide at a 4× dilution series). Flow cytometry was used to measure surface level expression of CD69 after 1 day of co-culture. Representative EC50 curves are shown in FIG. 2. These data demonstrate that the identified TCRs specific for GAD65 peptide are capable of activating CD4+ T cells even at low concentrations of GAD65 peptide.

The EC50 values (nM GAD65 peptide) the 9 TCRs in conventional CD4+ T cells, with and/or without disulfide modification are shown in Table 2.

TABLE 2

| | EC50 nanomolar | N |
|---|---|---|
| TCR | (nM) | (experimental replicates) |
| TCR-B | 57.5966667 | 6 |
| TCR-B DS | 41.515 | 2 |
| TCD-D | 26.87 | 2 |
| TCR-E | 16.93 | 2 |
| TCR-G | 26.2633333 | 3 |
| TCR-H | 23.31 | 2 |
| TCR-I | 10000 | 1 |
| TCR-J | 23.06 | 1 |
| TCR-K | 6.196 | 2 |
| TCR-M | 9.101 | 2 |

### Example 2. Antigen-Specific Regulatory T Cell Suppression Studies

Human regulatory T cells (Tregs) exert their anti-inflammatory functions in part through suppression of conventional T cell (Tconv) activation and proliferation. TCR-transduced Tregs (transduced with TCR-B DS, TCR-G, TCR-K DS, or TCR-M DS) can be activated in an antigen-specific manner by the presentation of GAD65 555-567 by HLA-DRB1*04:01⁺ antigen-presenting cells (APCs).

For antigen-specific suppression studies, both Tregs and Tconvs were transduced with GAD65 555-567 specific TCRs. Isolated CD4⁺ CD25⁺ CD127^{low} Tregs were transduced with TCR-B DS, TCR-G, TCR-K DS, or TCR-M DS. CD4⁺ CD25⁻ Tconv cells isolated from healthy donors were transduced with TCR-B DS. The percentage of TCR⁺ cells was assessed by flow cytometry measurement of GFP⁺ cells as the lentiviral vector encoding the TCRs included an integrated green fluorescent protein (GFP). The proportion of TCR⁺ cells was then normalized across samples. TCR-transduced Tregs and TCR-transduced Tconvs were co-cultured and stimulated with GAD65 555-567 peptide presented by HLA-DRB1*04:01⁺ PBMCs. After four days, cells were collected for flow cytometry analysis. The analysis was repeated with cells (Treg and Tconv) from three donors.

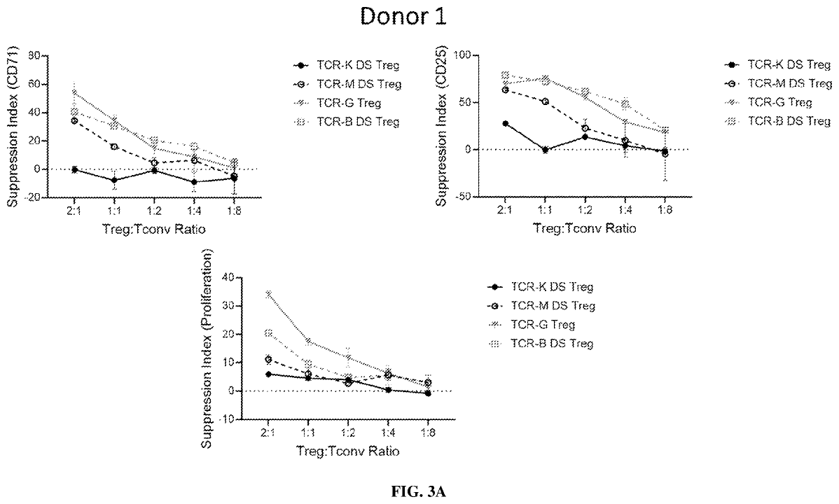

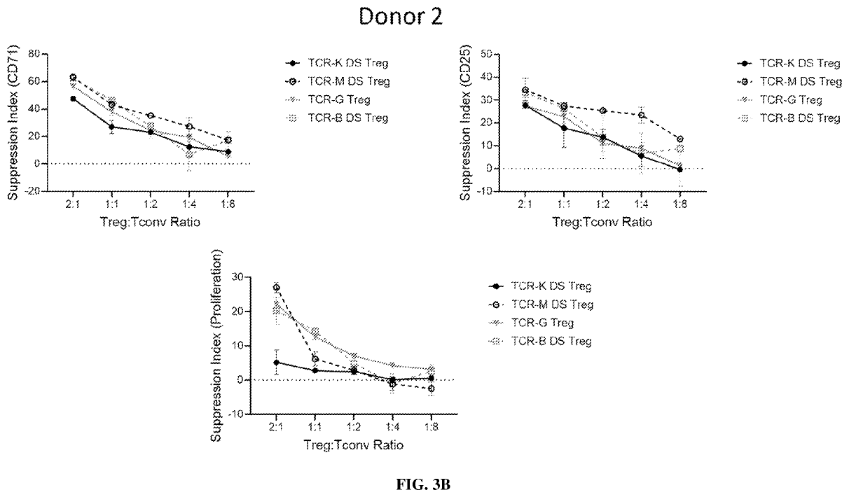

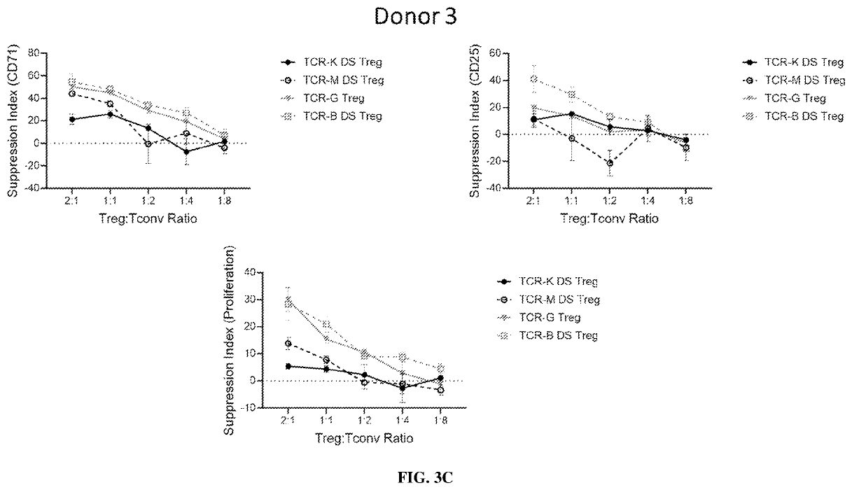

In response to antigen stimulation, the TCR-transduced Tregs suppressed Tconv activation as measured by the inhibition of activation markers CD25 and CD71; and suppressed Tconv cell proliferation as measured by CellTrace Violet dye dilution in cells from each of the three donors (FIGS. 3A-3C). Tregs transduced with TCR-B DS, TCR-G, TCR-K DS, or TCR-M DS suppressed proliferation and expression of activation associated markers CD71 and CD25, by GAD65 555-567 reactive Tconv at indicated Treg:Tconv ratios in the antigen-specific suppression study.

These data demonstrate that Tregs transduced with the GAD65-specific TCRs of the disclosure are capable of functioning, suppressing activation, proliferation and cytokine production of non-regulatory T cells (e.g., CD4⁺ conventional T cells).

Treg suppression of Tconv cell proliferation at indicated Treg:Tconv ratios was calculated as follows:

$$100 * \frac{\begin{matrix}[ Average \% of divided Tconv cells without Tregs ] - \\ [ \% of divided Tconv cells with Tregs ]\end{matrix}}{[ Average \% of divided Tconv cells without Tregs ]}$$

Treg suppression of Tconv cell activation at indicated Treg:Tconv ratios was calculated as follows:

$$100 * \frac{\begin{matrix}[ Average \% of activated Tconv cells without Tregs ] - \\ [ \% of activated Tconv cells with Tregs ]\end{matrix}}{[ Average \% of activated Tconv cells without Tregs ]}$$

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 61,
a CDR2-alpha sequence comprising the amino acid sequence of SEQ ID NO: 62,
a CDR3-alpha sequence comprising the amino acid sequence of SEQ ID NO: 63,
a CDR1-beta sequence comprising the amino acid sequence of SEQ ID NO: 66,
a CDR2-beta sequence comprising the amino acid sequence of SEQ ID NO: 67, and/or
a CDR3-beta sequence comprising the amino acid sequence of SEQ ID NO: 68.

2. The TCR of claim 1 comprising:
a CDR1-alpha sequence comprising the amino acid sequence of SEQ ID NO: 61,
a CDR2-alpha sequence comprising the amino acid sequence of SEQ ID NO: 62, and
a CDR3-alpha sequence comprising the amino acid sequence of SEQ ID NO: 63.

3. The TCR of claim 1 or 2, wherein the alpha chain comprises an alpha variable region comprising an amino acid sequence having at least 90%, 95%, or 100% identity to the amino acid sequence of SEQ ID NO: 64.

4. The TCR of claim 3, wherein the alpha chain comprises an amino acid sequence having at least 90%, 95%, or 100% identity to the amino acid sequence of SEQ ID NO: 65.

5. The TCR of claim 3, wherein the alpha chain comprises an amino acid sequence having at least 90%, 95%, or 100% identity to the amino acid sequence of SEQ ID NO: 272.

6. The TCR of any one of claims 1-5 comprising:
a CDR1-beta sequence comprising the amino acid sequence of SEQ ID NO: 66,
a CDR2-beta sequence comprising the amino acid sequence of SEQ ID NO: 67, and
a CDR3-alpha sequence comprising the amino acid sequence of SEQ ID NO: 68.

7. The TCR of claim 6, wherein the beta chain comprises a beta variable region comprising an amino acid sequence having at least 90%, 95%, or 100% identity to the amino acid sequence of SEQ ID NO: 69.

8. The TCR of claim 7, wherein the beta chain comprises an amino acid sequence having at least 90%, 95%, or 100% identity to the amino acid sequence of SEQ ID NO: 70.

9. The TCR of claim 7, wherein the beta chain comprises an amino acid sequence having at least 90%, 95%, or 100% identity to the amino acid sequence of SEQ ID NO: 273.

10. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 110,
a CDR2-alpha sequence comprising the amino acid sequence of SEQ ID NO: 111,
a CDR3-alpha sequence comprising the amino acid sequence of SEQ ID NO: 112,
a CDR1-beta sequence comprising the amino acid sequence of SEQ ID NO: 115,
a CDR2-beta sequence comprising the amino acid sequence of SEQ ID NO: 116, and/or
a CDR3-beta sequence comprising the amino acid sequence of SEQ ID NO: 117.

11. The TCR of claim 10 comprising:
a CDR1-alpha sequence comprising the amino acid sequence of SEQ ID NO: 110,
a CDR2-alpha sequence comprising the amino acid sequence of SEQ ID NO: 111, and
a CDR3-alpha sequence comprising the amino acid sequence of SEQ ID NO: 112.

12. The TCR of claim 10 or 11, wherein the alpha chain comprises an alpha variable region comprising an amino acid sequence having at least 90%, 95%, or 100% identity to the amino acid sequence of SEQ ID NO: 113.

13. The TCR of claim 12, wherein the alpha chain comprises an amino acid sequence having at least 90%, 95%, or 100% identity to the amino acid sequence of SEQ ID NO: 114.

14. The TCR of claim 12, wherein the alpha chain comprises an amino acid sequence having at least 90%, 95%, or 100% identity to the amino acid sequence of SEQ ID NO: 280.

15. The TCR of any one of claims 10-14 comprising:
a CDR1-beta sequence comprising the amino acid sequence of SEQ ID NO: 115,
a CDR2-beta sequence comprising the amino acid sequence of SEQ ID NO: 116, and
a CDR3-beta sequence comprising the amino acid sequence of SEQ ID NO: 117.

16. The TCR of claim 15, wherein the beta chain comprises a beta variable region comprising an amino acid sequence having at least 90%, 95%, or 100% identity to the amino acid sequence of SEQ ID NO: 118.

17. The TCR of claim 16, wherein the beta chain comprises an amino acid sequence having at least 90%, 95%, or 100% identity to the amino acid sequence of SEQ ID NO: 119.

18. The TCR of claim 16, wherein the beta chain comprises an amino acid sequence having at least 90%, 95%, or 100% identity to the amino acid sequence of SEQ ID NO: 281.

19. A T cell receptor (TCR) that binds to a GAD65 peptide complexed with a major histocompatibility complex (MHC) molecule, wherein the TCR comprises an alpha chain and a beta chain, and wherein the TCR comprises:
a CDR1-alpha sequence comprising the amino acid sequence of SEQ ID NO: 130,
a CDR2-alpha sequence comprising the amino acid sequence of SEQ ID NO: 131,
a CDR3-alpha sequence comprising the amino acid sequence of SEQ ID NO: 132,
a CDR1-beta sequence comprising the amino acid sequence of SEQ ID NO: 135,
a CDR2-beta sequence comprising the amino acid sequence of SEQ ID NO: 136, and/or
a CDR3-beta sequence comprising the amino acid sequence of SEQ ID NO: 137.

20. The TCR of claim 19 comprising:
a CDR1-alpha sequence comprising the amino acid sequence of SEQ ID NO: 130,
a CDR2-alpha sequence comprising the amino acid sequence of SEQ ID NO: 131, and
a CDR3-alpha sequence comprising the amino acid sequence of SEQ ID NO: 132.

21. The TCR of claim 19, wherein the alpha chain comprises an alpha variable region comprising an amino acid sequence having at least 90%, 95%, or 100% identity to the amino acid sequence of SEQ ID NO: 133.

22. The TCR of claim 21, wherein the alpha chain comprises an amino acid sequence having at least 90%, 95%, or 100% identity to the amino acid sequence of SEQ ID NO: 134.

23. The TCR of claim 21, wherein the alpha chain comprises an amino acid sequence having at least 90%, 95%, or 100% identity to the amino acid sequence of SEQ ID NO: 284.

24. The TCR of any one of claims 19-23 comprising:
a CDR1-beta sequence comprising the amino acid sequence of SEQ ID NO: 135,
a CDR2-beta sequence comprising the amino acid sequence of SEQ ID NO: 136, and
a CDR3-beta sequence comprising the amino acid sequence of SEQ ID NO: 137.

25. The TCR of claim 24, wherein the beta chain comprises a beta variable region comprising an amino acid sequence having at least 90%, 95%, or 100% identity to the amino acid sequence of SEQ ID NO: 138.

26. The TCR of claim 25, wherein the beta chain comprises an amino acid sequence having at least 90%, 95%, or 100% identity to the amino acid sequence of SEQ ID NO: 139.

27. The TCR of claim 25, wherein the beta chain comprises an amino acid sequence having at least 90%, 95%, or 100% identity to the amino acid sequence of SEQ ID NO: 285.

28. The TCR of any one of the preceding claims, wherein the GAD65 peptide is a GAD65 555-567 peptide comprising the amino acid sequence of NFFRMVISNPAAT (SEQ ID NO: 109).

29. The TCR of any one of the preceding claims, wherein the MHC comprises HLA-DRB1*04:01.

30. The TCR of any one of the preceding claims, wherein the TCR is encoded as a single polypeptide, optionally wherein the polypeptide comprises an N-terminal beta domain and a C-terminal alpha domain.

31. The TCR of claim 30, wherein the single polypeptide comprises a self-cleaving peptide sequence positioned between the alpha chain and the beta chain, optionally wherein the self-cleaving peptide sequence is a 2A peptide sequence, optionally wherein the 2A peptide sequence is a P2A, E2A, F2A, or T2A peptide sequence.

32. The TCR of any one of the preceding claims, wherein the alpha chain comprises an alpha constant region comprising an amino acid sequence having at least 90%, 95%, or 100% identity to the amino acid sequence of SEQ ID NO: 106.

33. The TCR of any one of the preceding claims, wherein the beta chain comprises a beta constant region comprising an amino acid sequence having at least 90%, 95%, or 100% identity to the amino acid sequence of SEQ ID NO: 107 or 108.

34. The TCR of any one of the preceding claims, wherein TCR comprises one or more amino acid substitutions to cysteine residues in the alpha chain constant region and the beta chain constant region, and wherein the cysteine residues are capable of forming one or more disulfide bridges.

35. The TCR of any one of the preceding claims, wherein the alpha chain constant region comprises a T48C amino acid substitution relative to an alpha chain constant region comprising the amino acid sequence of SEQ ID NO: 106, and wherein the beta chain constant region comprises a S57C amino acid substitution relative to a beta chain constant region comprising the amino acid sequence of SEQ ID NO: 108.

36. An engineered nucleic acid encoding the TCR of any one of the preceding claims.

37. The engineered nucleic acid of claim 36, wherein the nucleic acid is a vector.

38. The engineered nucleic acid of claim 37, wherein the vector is a viral vector.

39. The engineered nucleic acid of claim 38, wherein the viral vector is a lentiviral vector.

40. The engineered nucleic acid of any one of claims 36-39, wherein the nucleic acid comprises a promoter operably linked to a coding sequence encoding the TCR, optionally wherein the promoter is an EF-1 alpha promoter or an MND promoter.

41. The engineered nucleic acid of any one of claims 36-40, wherein the nucleic acid further comprises an enhancer element, optionally an optimized post-transcriptional regulatory element (oPRE) or a woodchuck hepatitis virus post-transcriptional regulatory element (WPRE), further optionally WPRE-mut6.

42. The engineered nucleic acid of any one of claims 36-41, wherein the coding sequence is codon-optimized.

43. An engineered cell comprising a TCR that specifically binds GAD65 555-567 complexed with a major histocompatibility complex (MHC) molecule, wherein the TCR is exogenous to the engineered cell.

44. The engineered cell of claim 43, wherein the TCR comprises the TCR of any one of claims 1-35 or the nucleic acid of any one of claims 36-42.

45. The engineered cell of claim 43 or 44, wherein the cell is a T cell, optionally a regulatory T cell.

46. An engineered regulatory T cell comprising a TCR that specifically binds GAD65 555-567 peptide, complexed with a major histocompatibility complex (MHC) molecule, wherein the TCR is exogenous to the engineered cell.

47. The engineered regulatory T cell of claim 46, wherein the TCR comprises the TCR of any one of claims 1-35 or the nucleic acid of any one of claims 36-42.

48. The engineered cell of any one of claims 43-47, wherein the cell is derived from a subject, optionally a subject having or suspected of having Type 1 Diabetes.

49. A cell population comprising a plurality of the engineered cell of any one of claims 43-48.

50. A pharmaceutical composition comprising the cell population of claim 49 and a pharmaceutically acceptable excipient.

51. A composition comprising the cell population of claim 49 and a cryopreservative.

52. A method comprising administering to a subject the cell population of claim 49, the pharmaceutical composition of claim 50, or the composition of claim 51, wherein the subject has Type 1 Diabetes.

53. The method of claim 52, wherein the cell population or the pharmaceutical composition, or the composition is administered in an effective amount to alleviate one or more symptom of Type 1 Diabetes.

54. The method of claim 52 or 53, wherein the administering comprises intravenous administration.

55. The method of any one of claims 52-54, wherein the administering comprises one or more infusion.


SEQUENCE LISTING (291 sequences)

SEQ ID NO: 1 - protein, 9 aa, synthetic construct
ETRDTTYYL

SEQ ID NO: 2 - protein, 10 aa, synthetic construct
RRNSFDEQNE

SEQ ID NO: 3 - protein, 16 aa, synthetic construct
CALSENRGGTASKLTF

SEQ ID NO: 4 - protein, 117 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSENRGGTASKLTFGTGTRLQVTL

SEQ ID NO: 5 - protein, 258 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSENRGGTASKLTFGTGTRLQVTLNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 6 - protein, 7 aa, synthetic construct
ISGHRSV

SEQ ID NO: 7 - protein, 8 aa, synthetic construct
EYFSETQR

SEQ ID NO: 8 - protein, 15 aa, synthetic construct
CASSLVGGPSSEAFF

SEQ ID NO: 9 - protein, 114 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLVGGPSSEAFFGQGTRLTVV

SEQ ID NO: 10 - protein, 293 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLVGGPSSEAFFGQGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 11 - protein, 9 aa, synthetic construct
ETRDTTYYL

SEQ ID NO: 12 - protein, 10 aa, synthetic construct
RRNSFDEQNE

SEQ ID NO: 13 - protein, 16 aa, synthetic construct
CALSEEGGGANSKLTF

SEQ ID NO: 14 - protein, 117 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEEGGGANSKLTFGKGITLSVRP

SEQ ID NO: 15 - protein, 258 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEEGGGANSKLTFGKGITLSVRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 7 aa, synthetic construct
ISGHRSV

SEQ ID NO: 17 - protein, 8 aa, synthetic construct
EYFSETQR

SEQ ID NO: 18 - protein, 15 aa, synthetic construct
CASSLAGGANSPLHF

SEQ ID NO: 19 - protein, 114 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLAGGANSPLHFGNGTRLTVT

SEQ ID NO: 20 - protein, 293 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLAGGANSPLHFGNGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 21 - protein, 7 aa, synthetic construct
SSIFNTW

SEQ ID NO: 22 - protein, 9 aa, synthetic construct
ALYKAGELT

SEQ ID NO: 23 - protein, 15 aa, synthetic construct
CAGHSIIQGAQKLVF

SEQ ID NO: 24 - protein, 113 aa, synthetic construct
GQQLNQSPQSMFIQEGEDVSMNCTSSSIFNTWLWYKQEPGEGPVLLIALYKAGELTSNGRLTAQFGITRKDSFLNISASIPSDVGIYFCAGHSIIQGAQKLVFGQGTRLTINP

SEQ ID NO: 25 - protein, 254 aa, synthetic construct
GQQLNQSPQSMFIQEGEDVSMNCTSSSIFNTWLWYKQDPGEGPVLLIALYKAGELTSNGRLTAQFGITRKDSFLNISASIPSDVGIYFCAGHSIIQGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 7 aa, synthetic construct
ISGHRSV

SEQ ID NO: 27 - protein, 8 aa, synthetic construct
EYFSETQR

SEQ ID NO: 28 - protein, 13 aa, synthetic construct
CASGRSSYNEQFF

SEQ ID NO: 29 - protein, 112 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASGRSSYNEQFFGPGTRLTVL

SEQ ID NO: 30 - protein, 291 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASGRSSYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - protein, 8 aa, synthetic construct
TATGYPSL

SEQ ID NO: 32 - protein, 9 aa, synthetic construct
KATKADDKG

SEQ ID NO: 33 - protein, 17 aa, synthetic construct
CALSDHAGGTSYGKLTF

SEQ ID NO: 34 - protein, 116 aa, synthetic construct
GDSVTQMEGPVTLSEEAFLTINCTYTATGYPSLFWYVQYPGEGLQLLLKATKADDKGSNKGFEATYRKETTSFHLEKGSVQVSDSAVYFCALSDHAGGTSYGKLTFGQGTILTVHP

SEQ ID NO: 35 - protein, 276 aa, synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSDHAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 36 - protein, 7 aa, synthetic construct
DMNHEYM

SEQ ID NO: 37 - protein, 8 aa, synthetic construct
YSVGAGIT

SEQ ID NO: 38 - protein, 14 aa, synthetic construct
CASMSPATNEKLFF

SEQ ID NO: 39 - protein, 113 aa, synthetic construct
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASMSPATNEKLFFGSGTQLSVL

SEQ ID NO: 40 - protein, 309 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASMSPATNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 41 - protein, 8 aa, synthetic construct
SSSVPPYL

SEQ ID NO: 42 - protein, 10 aa, synthetic construct
KYTSAATLVK

SEQ ID NO: 43 - protein, 15 aa, synthetic construct
CAVSDQNSGYSTLTF

SEQ ID NO: 44 - protein, 115 aa, synthetic construct
AQSVTQLGSHVSVSEGALVLLRCNYSSSVPPYLFWYVQYPNQGLQLLLKYTSAATLVKGINGFEAEFKKSETSFHLTKPSAHMSDAAEYFCAVSDQNSGYSTLTFGKGTMLLVSP

SEQ ID NO: 45 - protein, 275 aa, synthetic construct
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSDQNSGYSTLTFGKGTMLLVSPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 46 - protein, 7 aa, synthetic construct
ISGHRSV

SEQ ID NO: 47 - protein, 8 aa, synthetic construct
EYFSETQR

SEQ ID NO: 48 - protein, 15 aa, synthetic construct
CASSFKGATNEKLFF

SEQ ID NO: 49 - protein, 114 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSFKGATNEKLFFGSGTQLSVL

SEQ ID NO: 50 - protein, 310 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFKGATNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 51 - protein, 8 aa, synthetic construct
TNYSPAYL

SEQ ID NO: 52 - protein, 9 aa, synthetic construct
LIRENEKEK

SEQ ID NO: 53 - protein, 16 aa, synthetic construct
CALGGVGSGGYNKLIF

SEQ ID NO: 54 - protein, 115 aa, synthetic construct
SQKIEQNSEALNIQEGKTATLTCNYTNYSPAYLQWYRQDPGRGPVFLLLIRENEKEKRKERLKVTFDTTLKQSLFHITASQPADSATYLCALGGVGSGGYNKLIFGAGTRLAVHP

SEQ ID NO: 55 - protein, 276 aa, synthetic construct
MESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALGGVGSGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 56 - protein, 7 aa, synthetic construct
HMGHRAM

SEQ ID NO: 57 - protein, 8 aa, synthetic construct
VYSYEKLS

SEQ ID NO: 58 - protein, 14 aa, synthetic construct
CASSQDNHAGELFF

SEQ ID NO: 59 - protein, 113 aa, synthetic construct
DTEVTQTPKHLVMGMTNKKSLKCEQHMGHRAMYWYKQKAKKPPELMFVYSYEKLSINESVPSRFSPECPNSSLLNLHLHALQPEDSALYLCASSQDNHAGELFFGEGSRLTVL

SEQ ID NO: 60 - protein, 311 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQDNHAGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 61 - protein, 8 aa, synthetic construct
TATGYPSL

SEQ ID NO: 62 - protein, 9 aa, synthetic construct
KATKADDKG

SEQ ID NO: 63 - protein, 15 aa, synthetic construct
CALSDPGGGSYIPTF

SEQ ID NO: 64 - protein, 114 aa, synthetic construct
GDSVTQMEGPVTLSEEAFLTINCTYTATGYPSLFWYVQYPGEGLQLLLKATKADDKGSNKGFEATYRKETTSFHLEKGSVQVSDSAVYFCALSDPGGGSYIPTFGRGTSLIVHP

SEQ ID NO: 65 - protein, 274 aa, synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSDPGGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 66 - protein, 7 aa, synthetic construct
TLNHNVM

SEQ ID NO: 67 - protein, 8 aa, synthetic construct
HYYDKDFN

SEQ ID NO: 68 - protein, 15 aa, synthetic construct
CATSRAGDDLALQFF

SEQ ID NO: 69 - protein, 114 aa, synthetic construct
DAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRAGDDLALQFFGPGTRLTVL

SEQ ID NO: 70 - protein, 312 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRAGDDLALQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 71 - protein, 8 aa, synthetic construct
QTSGFYGL

SEQ ID NO: 72 - protein, 8 aa, synthetic construct
YNALDGLE

SEQ ID NO: 73 - protein, 15 aa, synthetic construct
CAVKKGSSNTGKLIF

SEQ ID NO: 74 - protein, 112 aa, synthetic construct
GQSLEQPSEVTAVEGAIVQINCTYQTSGFYGLSWYQQHDGGAPTFLSYNALDGLEETGRFSSFLSRSDSYGYLLLQELQMKDSASYFCAVKKGSSNTGKLIFGQGTTLQVKP

SEQ ID NO: 75 - protein, 270 aa, synthetic construct
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVKKGSSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 76 - protein, 7 aa, synthetic construct
ISGHDNL

SEQ ID NO: 77 - protein, 8 aa, synthetic construct
HFVKESKQ

SEQ ID NO: 78 - protein, 13 aa, synthetic construct
CASSPLQGYEQYF

SEQ ID NO: 79 - protein, 113 aa, synthetic construct
EAGVTQFPSHSVIEKGQTVTLRCDPISGHDNLYWYRRVMGKEIKFLLHFVKESKQDESGMPNNRFLAERTGGTYSTLKVQPAELEDSGVYFCASSPLQGYEQYFGPGTRLTVT

SEQ ID NO: 80 - protein, 311 aa, synthetic construct
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSPLQGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 81 - protein, 8 aa, synthetic construct
SNSASQSF

SEQ ID NO: 82 - protein, 7 aa, synthetic construct
SVYSSGN

SEQ ID NO: 83 - protein, 12 aa, synthetic construct
CVVNRRNARLMF

SEQ ID NO: 84 - protein, 109 aa, synthetic construct
RKEVEQDPGPFNVPEGATVAFNCTYSNSASQSFFWYRQDCRKEPKLLMSVYSSGNEDGRFTAQLNRASQYISLLIRDSKLSDSATYLCVVNRRNARLMFGDGTQLVVKP

SEQ ID NO: 85 - protein, 271 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNRRNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 86 - protein, 7 aa, synthetic construct
TKGHDRM

SEQ ID NO: 87 - protein, 8 aa, synthetic construct
YSFDVKDI

SEQ ID NO: 88 - protein, 14 aa, synthetic construct
CATSDGDGQETQYF

SEQ ID NO: 89 - protein, 113 aa, synthetic construct
DADVTQTPRNRITKTGKRIMLECSQTKGHDRMYWYRQDPGLGLQLIYYSFDVKDINKGEISDGYSVSRQAQAKFSLSLESAIPNQTALYFCATSDGDGQETQYFGPGTRLLVL

SEQ ID NO: 90 - protein, 311 aa, synthetic construct
MASLLFFCGAFYLLGTGSMDADVTQTPRNRITKTGKRIMLECSQTKGHDRMYWYRQDPGLGLQLIYYSFDVKDINKGEISDGYSVSRQAQAKFSLSLESAIPNQTALYFCATSDGDGQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 91 - protein, 8 aa, synthetic construct
TNYSPAYL

SEQ ID NO: 92 - protein, 9 aa, synthetic construct
LIRENEKEK

SEQ ID NO: 93 - protein, 13 aa, synthetic construct
CAPISGSARQLTF

SEQ ID NO: 94 - protein, 112 aa, synthetic construct
SQKIEQNSEALNIQEGKTATLTCNYTNYSPAYLQWYRQDPGRGPVFLLLIRENEKEKRKERLKVTFDTTLKQSLFHITASQPADSATYLCAPISGSARQLTFGSGTQLTVLP

SEQ ID NO: 95 - protein, 273 aa, synthetic construct
MESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCAPISGSARQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 96 - protein, 7 aa, synthetic construct
NLNHDAM

SEQ ID NO: 97 - protein, 8 aa, synthetic construct
YSQIVNDF

SEQ ID NO: 98 - protein, 13 aa, synthetic construct
CASSMGQGQPQHF

SEQ ID NO: 99 - protein, 112 aa, synthetic construct
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSMGQGQPQHFGDGTRLSIL

SEQ ID NO: 100 - protein, 308 aa, synthetic construct
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSMGQGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 101 - protein, 8 aa, synthetic construct
DXEXNPGP

SEQ ID NO: 102 - protein, 18 aa, synthetic construct
EGRGSLLTCGDVEENPGP

SEQ ID NO: 103 - protein, 19 aa, synthetic construct
ATNFSLLKQAGDVEENPGP

SEQ ID NO: 104 - protein, 20 aa, synthetic construct
QCTNYALLKLAGDVESNPGP

SEQ ID NO: 105 - protein, 22 aa, synthetic construct
VKQTLNFDLLKLAGDVESNPGP

SEQ ID NO: 106 - protein, 141 aa, synthetic construct
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 107 - protein, 177 aa, synthetic construct
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 108 - protein, 179 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 109 - protein, 13 aa, synthetic construct
NFFRMVISNPAAT

SEQ ID NO: 110 - protein, 9 aa, synthetic construct
STISGTDYI

SEQ ID NO: 111 - protein, 7 aa, synthetic construct
HGLTSNV

SEQ ID NO: 112 - protein, 15 aa, synthetic construct
CILRDVENYGQNFVF

SEQ ID NO: 113 - protein, 112 aa, synthetic construct
DAKTTQPNSMESNEEEPVHLPCNHSTISGTDYIHWYRQLPSQGPEYVIHGLTSNVNNRMASLAIAEDRKSSTLILHRATLRDAAVYYCILRDVENYGQNFVFGPGTRLSVLP

SEQ ID NO: 114 - protein, 270 aa, synthetic construct
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILRDVENYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 115 - protein, 7 aa, synthetic construct
ISEHNRL

SEQ ID NO: 116 - protein, 8 aa, synthetic construct
YFQNEAQL

SEQ ID NO: 117 - protein, 15 aa, synthetic construct
CASSLWTSGNSPLHF

SEQ ID NO: 118 - protein, 115 aa, synthetic construct
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLWTSGNSPLHFGNGTRLTVT

SEQ ID NO: 119 - protein, 311 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLWTSGNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 120 - protein, 7 aa, synthetic construct
STTLSNI

SEQ ID NO: 121 - protein, 9 aa, synthetic construct
QLVKSGEVK

SEQ ID NO: 122 - protein, 13 aa, synthetic construct
CAPPQTGANNLFF

SEQ ID NO: 123 - protein, 111 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAPPQTGANNLFFGTGTRLTVIP

SEQ ID NO: 124 - protein, 270 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAPPQTGANNLFFGTGTRLTVIPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 125 - protein, 7 aa, synthetic construct
ISGHRSV

SEQ ID NO: 126 - protein, 8 aa, synthetic construct
EYFSETQR

SEQ ID NO: 127 - protein, 15 aa, synthetic construct
CASSLVGGGDTEAFF

SEQ ID NO: 128 - protein, 114 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLVGGGDTEAFFGQGTRLTVV

SEQ ID NO: 129 - protein, 310 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLVGGGDTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 130 - protein, 8 aa, synthetic construct
TNYSPAYL

SEQ ID NO: 131 - protein, 9 aa, synthetic construct
LIRENEKEK

SEQ ID NO: 132 - protein, 14 aa, synthetic construct
CALPTTDSWGKLQF

SEQ ID NO: 133 - protein, 113 aa, synthetic construct
SQKIEQNSEALNIQEGKTATLTCNYTNYSPAYLQWYRQDPGRGPVFLLLIRENEKEKRKERLKVTFDTTLKQSLFHITASQPADSATYLCALPTTDSWGKLQFGAGTQVVVTP

SEQ ID NO: 134 - protein, 274 aa, synthetic construct
MESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALPTTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 135 - protein, 7 aa, synthetic construct
ISGHRSV

SEQ ID NO: 136 - protein, 8 aa, synthetic construct
EYFSETQR

SEQ ID NO: 137 - protein, 14 aa, synthetic construct
CASSFKDREETQYF

SEQ ID NO: 138 - protein, 113 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSFKDREETQYFGPGTRLLVL

SEQ ID NO: 139 - protein, 311 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFKDREETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 140 - protein, 8 aa, synthetic construct
SNSASDYF

SEQ ID NO: 141 - protein, 9 aa, synthetic construct
DIRSNMDKR

SEQ ID NO: 142 - protein, 13 aa, synthetic construct
CAENGGNNARLMF

SEQ ID NO: 143 - protein, 112 aa, synthetic construct
GESVGLHLPTLSVQEGDNSIINCAYSNSASDYFIWYKQESGKGPQFIIDIRSNMDKRQGQRVTVLLNKTVKHLSLQIAATQPGDSAVYFCAENGGNNARLMFGDGTQLVVKP

SEQ ID NO: 144 - protein, 273 aa, synthetic construct
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENGGNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 145 - protein, 7 aa, synthetic construct
ISGHRSV

SEQ ID NO: 146 - protein, 8 aa, synthetic construct
EYFSETQR

SEQ ID NO: 147 - protein, 14 aa, synthetic construct
CASSATGGDQPQHF

SEQ ID NO: 148 - protein, 113 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSATGGDQPQHFGDGTRLSIL

SEQ ID NO: 149 - protein, 309 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSATGGDQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 150 - protein, 7 aa, synthetic construct
STTSDRL

SEQ ID NO: 151 - protein, 9 aa, synthetic construct
VLLSNGAVK

SEQ ID NO: 152 - protein, 15 aa, synthetic construct
CAVDMTGSSGDKLTF

SEQ ID NO: 153 - protein, 113 aa, synthetic construct
ELKVEQNPLFLSMQEGKNYTIYCNYSTTSDRLYWYRQDPGKSLESLFVLLSNGAVKQEGRLMASLDTKARLSTLHITAAVHDLSATYFCAVDMTGSSGDKLTFGTGTRLAVRP

SEQ ID NO: 154 - protein, 272 aa, synthetic construct
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAVDMTGSSGDKLTFGTGTRLAVRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 155 - protein, 7 aa, synthetic construct
ISGHVSL

SEQ ID NO: 156 - protein, 8 aa, synthetic construct
YFQNEAQL

SEQ ID NO: 157 - protein, 16 aa, synthetic construct
CASSLVAGHGGDGYTF

SEQ ID NO: 158 - protein, 116 aa, synthetic construct
GAGVSQSPRYKVAKRGQDVALRCDPISGHVSLFWYQQALGQGPEFLTYFQNEAQLDKSGLPSDRFFAERPEGSVSTLKIQRTQKEDSAVYLCASSLVAGHGGDGYTFGSGTRLTVV

SEQ ID NO: 159 - protein, 312 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQKEDSAVYLCASSLVAGHGGDGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 160 - protein, 7 aa, synthetic construct
STTLSNI

SEQ ID NO: 161 - protein, 9 aa, synthetic construct
QLVKSGEVK

SEQ ID NO: 162 - protein, 13 aa, synthetic construct
CAGLGGSNYKLTF

SEQ ID NO: 163 - protein, 111 aa, synthetic construct
GQQVMQIPQYQHVQEGEDFTTYCNSSTTLSNIQWYKQRPGGHPVFLIQLVKSGEVKKQKRLTFQFGEAKKNSSLHITATQTTDVGTYFCAGLGGSNYKLTFGKGTLLTVNP

SEQ ID NO: 164 - protein, 270 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGLGGSNYKLTFGKGTLLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 165 - protein, 7 aa, synthetic construct
NLNHDAM

SEQ ID NO: 166 - protein, 8 aa, synthetic construct
YSQIVNDF

SEQ ID NO: 167 - protein, 13 aa, synthetic construct
CASSPFSTDTQYF

SEQ ID NO: 168 - protein, 112 aa, synthetic construct
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPFSTDTQYFGPGTRLTVL

SEQ ID NO: 169 - protein, 310 aa, synthetic construct
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPFSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 170-259 - skipped (90 entries)

SEQ ID NO: 260 - protein, 258 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSENRGGTASKLTFGTGTRLQVTLNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 261 - protein, 293 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLVGGPSSEAFFGQGTRLTVVEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 262 - protein, 258 aa, synthetic construct
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEEGGGANSKLTFGKGITLSVRPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 263 - protein, 293 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLAGGANSPLHFGNGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 264 - protein, 254 aa, synthetic construct
GQQLNQSPQSMFIQEGEDVSMNCTSSSIFNTWLWYKQDPGEGPVLLIALYKAGELTSNGRLTAQFGITRKDSFLNISASIPSDVGIYFCAGHSIIQGAQKLVFGQGTRLTINPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 265 - protein, 291 aa, synthetic construct
KAGVTQTPRYLIKTRGQQVTLSCSPISGHRSVSWYQQTPGQGLQFLFEYFSETQRNKGNFPGRFSGRQFSNSRSEMNVSTLELGDSALYLCASGRSSYNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 266 - protein, 276 aa, synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSDHAGGTSYGKLTFGQGTILTVHPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 267 - protein, 309 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASMSPATNEKLFFGSGTQLSVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 268 - protein, 275 aa, synthetic construct
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSDQNSGYSTLTFGKGTMLLVSPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 269 - protein, 310 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFKGATNEKLFFGSGTQLSVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 270 - protein, 276 aa, synthetic construct
MESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALGGVGSGGYNKLIFGAGTRLAVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 271 - protein, 311 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQDNHAGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 272 - protein, 274 aa, synthetic construct
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSDPGGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 273 - protein, 312 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRAGDDLALQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 274 - protein, 270 aa, synthetic construct
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVKKGSSNTGKLIFGQGTTLQVKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 275 - protein, 311 aa, synthetic construct
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSPLQGYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 276 - protein, 271 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNRRNARLMFGDGTQLVVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 277 - protein, 311 aa, synthetic construct
MASLLFFCGAFYLLGTGSMDADVTQTPRNRITKTGKRIMLECSQTKGHDRMYWYRQDPGLGLQLIYYSFDVKDINKGEISDGYSVSRQAQAKFSLSLESAIPNQTALYFCATSDGDGQETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 278 - protein, 273 aa, synthetic construct
MESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCAPISGSARQLTFGSGTQLTVLPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 279 - protein, 308 aa, synthetic construct
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSMGQGQPQHFGDGTRLSILEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 280 - protein, 270 aa, synthetic construct
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILRDVENYGQNFVFGPGTRLSVLPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 281 - protein, 311 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLWTSGNSPLHFGNGTRLTVTEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 282 - protein, 270 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAPPQTGANNLFFGTGTRLTVIPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 283 - protein, 310 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLVGGGDTEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 284 - protein, 274 aa, synthetic construct
MESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALPTTDSWGKLQFGAGTQVVVTPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 285 - protein, 311 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFKDREETQYFGPGTRLLVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 286 - protein, 273 aa, synthetic construct
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENGGNNARLMFGDGTQLVVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 287 - protein, 309 aa, synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSATGGDQPQHFGDGTRLSILEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 288 - protein, 272 aa, synthetic construct
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAVDMTGSSGDKLTFGTGTRLAVRPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 289 - protein, 312 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQKEDSAVYLCASSLVAGHGGDGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 290 - protein, 270 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGLGGSNYKLTFGKGTLLTVNPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 291 - protein, 310 aa, synthetic construct
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPFSTDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG